(19) United States
(12) Patent Application Publication
(10) Pub. No.: US 2025/0120644 A1
(43) Pub. Date: Apr. 17, 2025

(54) Cognitive Screening Test for Detecting Preclinical Alzheimer's Disease

(71) Applicant: Cognivue, Inc., Victor, NY (US)

(72) Inventor: James E. Galvin, Victor, NY (US)

(21) Appl. No.: 18/913,545

(22) Filed: Oct. 11, 2024

Related U.S. Application Data
(60) Provisional application No. 63/658,721, filed on Jun. 11, 2024.
(60) Provisional application No. 63/591,001, filed on Oct. 17, 2023.
(60) Provisional application No. 63/590,253, filed on Oct. 13, 2023.

(51) Int. Cl. A61B 5/00; A61B 5/16; G16H 50/20

(52) U.S. Cl. CPC A61B 5/4088; A61B 5/163; G16H 50/20

(57) ABSTRACT

The Cognivue Test subtest scores may detect Preclinical Alzheimer's Disease (AD). This effect appears to be explained by: Adaptive Motor Response, Visual Salience, Shape Discrimination, and Visual Motor Reaction Time. Notably, the Preclinical AD group is also different from mild cognitive impairment MCI/AD and non-AD impairment across all tests (except Visual Motor Reaction Time) supporting the premise that the Cognivue Test can discriminate impaired individuals of any etiology from non-impaired individuals and further can identify non-impaired individuals who have amyloid deposition.

### FIELD OF THE INVENTION

The present invention relates to tests for evaluating cognitive function; and more particularly relates to screening tests for evaluating and discriminating cognitively impaired individuals of any etiology from non-impaired individuals, and still more particularly to screening tests for identifying non-impaired individuals who have amyloid deposition or preclinical Alzheimer's Disease.

### BACKGROUND OF THE INVENTION

Substantial literature exists describing sensory, cognitive, affective, and motor impairments due to neurological, neuropsychiatric, and psychiatric disorders. Sensory, cognitive, affective, and motor processing is impaired by brain dysfunction. However, many such abnormalities are unlikely to be uncovered during routine clinical and clinical laboratory examinations.

A method and system for quantitative assessment of functional impairment facilitates the detection and diagnosis of a variety of neurological diseases and disorders. A system for sensory-cognitive, affective, and motor quantitative neural assessment provides continuous feedback adjusted stimulation and its standardized scoring algorithms may provide for the detection of the early stages of brain changes and impairments associated with a variety of diseases and disorders. Quantitative assessment may aid in the investigation of sensory, cognitive, affective, and motor functions at various levels, including, but not limited to: sensory sensitivities to the variety of parametric variables affecting stimuli; for example, but not limited to, motion, object, depth, orientation, faces/expressions, hands/gestures, and other categorical classes of visual stimuli; perceived in the context of the comparison, detection, discrimination, recognition, and differentiation of the types of information analyzed by neural processing.

Further, quantitative assessment may indicate the detection, diagnosis, and distinguishing of a wide variety of health issues including, but not limited to, neurological, psychiatric, neuropsychiatric, psychological, neuropsychological, sensory, and motor diseases and disorders. These may reflect single or combined pathological, pathogenetic, and pathophysiological mechanisms including, but not limited to, congenital, demyelinating, infectious, metabolic, neoplastic, systemic, traumatic, and vascular effects. These include, but are not limited to, the well-known specific disease entities of Alzheimer's disease and other dementias, Parkinson's disease and other movement disorders, autism spectrum and other neuropsychiatric disorders, mood and other psychiatric disorders, and social maladjustment and other psychological disorders.

Other tools, such as behavioral assessments, cognitive testing, neurophysiological, and neuroimaging modalities have drawbacks related to difficulties in their consistent application, implementation, and interpretation. Paper and pencil tests, and their computer presentation and scoring tests do not consistently consider the results of initial tests in the arrangement and presentation of subsequent tests.

Additionally, since sensory, cognitive, affective, and motor impairments have not been universally recognized as closely linked, psychophysical neurobehavioral testing has not commonly been conducted during routine medical evaluations. Thus, a need exists, therefore, for developing appropriate tests to quantify the impact of related disorders. Further, although some consider neurobehavioral analysis to not be quantifiable, many research studies indicate that functional impairment can indeed be analyzed in a quantitative fashion. Thus, a further need exists for improved systems for the quantitative assessment of functional impairments to treat subjects with diseases, disorders, and dysfunction affecting sensory, perceptual, cognitive, and affective impairments, deficiencies, or disorders.

Yet a further need exists to identify the early phases of the neurobehavioral disease or disorder.

A further need exists for improved monitoring neurobehavioral disease progress.

Yet a further need exists for quantitative assessment of functional impairment that has the ability to simplify clinical research on sensory, cognitive, affective, motor including studies of perceptual, memory, attention, executive, and higher-order processing deficiencies.

Still further improvement is needed in animal research evaluations wherein quantitatively controlled variations in sensory stimuli and motor tasks are shown to animal subjects for the purposes of research, in basic and clinical science, leading to veterinary and medical testing of diagnostic, therapeutic, and other interventions.

Yet a further need exists for laboratories of drug and device companies and research facilities to research and develop treatments for functional impairment testing of human and animal subjects.

Still further improvement is needed to identify meta-parameters that may cause functional impairment and methods to diagnose their exemplary diseases and disorders.

A further need exists to generate real-time scores and diagnosis based on quantitative assessment of functional impairment.

Still further improvement is needed in critical testing of memory, attention, organizational, emotional, and social cue analysis.

A need exists for a treatment of development processes that may cause functional impairment in human subjects.

Yet a further need exists for maximizing stimulus response compatibility in assessment of functional impairment so as not to obscure aspects of neural processing.

Still further improvement is needed in a functional impairment assessment tool that captures all aspects of sensory input, cognitive transformation, affective interpretation, and motoric response.

Further, a need exists for the incorporation of artificial intelligence, that is the machine implementation of subject performance and characteristic data in the real-time parametric control of automated assessments of functional competence and impairment.

Likewise, a need exists for dynamic testing in clinical research, wherein a system responds to the actions of a subject.

Still further, there is a need for a less expensive, non-invasive screening tool for evaluating cognitive function to distinguish cognitively impaired individuals from non-impaired individuals, as well as identify non-impaired individuals who have amyloid deposition or preclinical Alzheimer's Disease.

The present invention addresses these and other needs.

### BRIEF SUMMARY OF THE INVENTION

The present invention relates to a method for quantitative assessment of functional impairment in an animal or human subject, where the method presents visual scenes, cues, and auditory locations or features to a subject, determines an equilibrated scene parameter of a subject, and generates output. For example, output may include the assessment of attention in Attention Deficits disorders in which the administration, titration, or discontinuation of stimulant and other specific pharmacotherapeutics, supplements, behavioral therapeutics, sensory or electrical stimulation or surgical intervention might be in part or entirely be directed based on these and related assessments of function. Or the assessment of memory in late-life dementias such as, but not limited to, cerebrovascular or neurodegenerative diseases in which the administration, titration, or discontinuation of a nootropic and other specific pharmacotherapeutics, supplements, behavioral therapeutics, sensory or electrical stimulation or surgical intervention might be in part or entirely be directed based on these and related assessments of function might be in part or entirely directed based on these and related assessments of function.

One aspect of the present disclosure includes an apparatus for quantifying assessment of functional impairment in a subject comprising an input device or devices, a visual, auditory, or tactile stimulation devices, a control device, and a tangible paper or computer readable output medium.

One aspect of the present disclosure includes a system for performing functional impairment tests that may continuously modulate specific perceptual domains of a stimulus and transition across perceptual domains in a manner to measure the response error relative to a specifically tested, individual or group, established or extrapolated normal range performance characteristics. In a simplified embodiment, an assessment profile of functional capacity by psychophysical responses is generated on a tangible computer readable medium.

The present disclosure improves and simplifies complex experimental paradigms in the context of behavioral, psychophysical, electrophysiological, and imaging studies of featural, spatial, temporal, and other categorically or parametrically manipulated aspects of function and its potential impairment or range of non-impaired variability within or between human and animal test subjects.

In accordance with the disclosed subject matter, the quantification of the impact of neural diseases onto affected sensory-cognitive-affective-motor functions is provided, thereby substantially advancing or facilitating the diagnosis and identification of the early and subsequent phases of neural diseases and disorders, as well as with secondary (after diagnosis) and tertiary (after initial therapy) prevention of the consequences of such diseases and disorders.

A need exists for developing appropriate tests to better understand neurobehavioral deficiencies. The present disclosure teaches a plurality of tests comprising a series of sensory stimulus arrays. More specifically, the present disclosure generates and presents complex dynamic scenes, collects responses from a human or animal test subject or patient, quantitatively refines results, calibrates a display device relative to the interpreted feedback, and provides clinically useful information regarding said subject or patient in the determination of the diagnosis and treatment of said subject or patient.

These and other advantages of the disclosed subject matter, as well as additional novel features, will be apparent from the description provided herein and from the attached figures. The intent of this summary is not to be a comprehensive or exhaustive description of the claimed subject matter, but rather to provide a short overview of exemplary instances and applications of the subject matter's functionality.

The invention addresses the above-referenced needs as well as other needs by providing a set of screening tests that together are used to detect preclinical AD. Generally, one aspect of the present invention is directed to a cognitive screening test comprising Adaptive Motor Response, Visual Salience, Shape Discrimination, and Visual Motor Reaction Time subtest for detecting preclinical Alzheimer's Disease.

A further aspect of the present invention is directed toward a method of detecting preclinical AD, which combines the following screening tests: adaptive motor response; visual salience; shape discrimination and visual motor reaction time, using the combined output of the four screening tests to detect preclinical AD. In still another aspect of the present invention, a computing system is configured to perform the method.

Additional objects, advantages and novel features of the present invention will be set forth in part in the description which follows, and will in part become apparent to those in the practice of the invention, when considered with the attached figures.

### BRIEF DESCRIPTION OF THE DRAWINGS

The accompanying drawings form a part of this specification and are to be read in conjunction therewith, wherein like reference numerals are employed to indicate like parts in the various views, and wherein:

The patent or application file contains at least one drawing executed in color. Copies of this patent or patent application publication with color drawing(s) will be provided by the Office upon request and payment of the necessary fee.

The present subject matter will now be described in detail with reference to the drawings, which are provided as illustrative examples of the subject matter so as to enable those skilled in the art to practice the subject matter. Notably, the figures and examples are not meant to limit the scope of the present subject matter to a single embodiment, but other embodiments are possible by way of interchange of some or all of the described or illustrated elements and, further, wherein:

FIG. 1 is a simplified schematic illustration showing aspects of a method of automated functional impairment testing in an embodiment.

FIG. 2 is a simplified schematic illustration showing aspects of a method of automated functional impairment testing in an embodiment.

FIG. 3 is a simplified illustration showing aspects of a system for automated functional impairment testing.

FIG. 4 is a simplified schematic diagram showing aspects of a computing system that may be used in a system for automated functional impairment testing according to an embodiment.

FIG. 5A is a simplified block diagram illustrating aspects of a system for automated functional impairment testing in an embodiment.

FIG. 5B is a simplified block diagram illustrating aspects of a method for automated functional impairment testing in an embodiment.

FIG. 5C is a simplified block diagram illustrating aspects of a system for automated functional impairment testing in an embodiment.

FIG. 5D is a simplified schematic diagram illustrating aspects of a system for automated functional impairment testing in an embodiment.

FIG. 5E is a simplified flow diagram illustrating aspects of a method for automated functional impairment testing in an embodiment.

FIG. 5F is a simplified block diagram illustrating aspects of a system for automated functional impairment testing in an embodiment.

FIG. 6 is a representation of left posterior-lateral view of the human brain.

FIG. 7 shows an exemplary operator display in a system for automated functional impairment testing.

FIG. 8 is a simplified illustration of physical components in a system for automated functional impairment testing.

FIG. 9 is simplified illustration of a rotary manipulandum test subject response device in a system for automated functional impairment testing.

FIG. 10 is a simplified illustration of a linear manipulandum test subject response device in a system for automated functional impairment testing.

FIG. 11 is a simplified illustration of an XY Cartesian manipulandum test subject response device in a system for automated functional impairment testing.

FIG. 12 is a simplified block diagram illustrating aspects of a stimulus generator including hardware and software for producing scene parameters in a system for automated functional impairment testing.

FIG. 13 is a simplified illustration of manual input components in a system for automated functional impairment testing.

FIG. 14 is a simplified illustration of an operator interface in a system for automated functional impairment testing in an embodiment.

FIG. 15 depicts an exemplary scoring output in a system for automated functional impairment testing.

FIG. 16 is an enlarged view of an exemplary operator display shown generally in FIG. 7 and showing a graphical user interface for a subject demographics entry interface display.

FIG. 17 is an enlarged view of an exemplary operator display similar to FIG. 16 and showing a graphical user interface for a subject medical history entry display.

FIG. 18A illustrates an exemplary operator display in a system for automated functional impairment testing.

FIG. 18B illustrates an exemplary standard operations test scoring display in a system for automated functional impairment testing.

FIG. 19A is an enlarged simplified illustration of an operator interface in a system for automated functional impairment testing, as shown generally in FIG. 14.

FIG. 19B is an enlarged simplified illustration of a graphical display showing Current Test Performance for an operator interface as shown generally in FIG. 14.

FIG. 20A illustrates an exemplary standard operations dynamic performance display of an operator interface.

FIG. 20B is an enlarged illustration of an exemplary operator comments entry display as shown general in FIG. 20A.

FIG. 21 is a simplified process flow diagram illustrating aspects of a system initiation sequence and a test initiation sequence in a system for automated functional impairment testing.

FIG. 22 is a simplified process flow diagram illustrating aspects of a test control sequence and a test presentation sequence in a system for automated functional impairment testing.

FIG. 23 is a simplified process flow diagram illustrating aspects of a test sequencing and test closing sequence in a system for automated functional impairment testing.

FIG. 24 is a simplified process flow diagram illustrating aspects of data archiving, operator interface and accounts management in a system for automated functional impairment testing.

FIG. 25 shows starting phase of a visual saliency test, in this case, an exemplary dynamic contrast test.

FIG. 26 illustrates the intermediate phase of a visual saliency test, in this case, an exemplary the dynamic contrast test of FIG. 25.

FIG. 27 displays the termination phase of a visual saliency test, in this case, an exemplary the dynamic contrast test of FIGS. 25 and 26.

FIG. 28 shows starting phase of a visual contrast sensitivity test.

FIG. 29 illustrates the intermediate phase of the visual contrast sensitivity test of FIG. 28.

FIG. 30 displays the termination phase of the visual contrast sensitivity test of FIGS. 28 and 29.

FIG. 31 portrays the starting phase of a visual motion discrimination test.

FIG. 32 shows the intermediate phase of the visual motion discrimination test of FIG. 31.

FIG. 33 illustrates the termination phase of the visual motion discrimination test of FIGS. 31 and 32.

FIG. 34 depicts the initiation of a visual motion discrimination test.

FIG. 35 shows the intermediate phase of the visual motion discrimination test of FIG. 34.

FIG. 36 illustrates the termination phase of the visual motion discrimination test of FIGS. 34 and 35.

FIG. 37 depicts the superposition of visual motion and visual form attention tests.

FIG. 38 illustrates the intermediate phase of a visual motion and visual form attention test of FIG. 37.

FIG. 39 represents the left-up form target and right-up motion target of a visual motion and visual form attention test of FIGS. 37 and 38.

FIG. 40 displays the left-up form, low-distinct target and right-up motion, high-coherence target of the visual motion and visual form attention test of FIGS. 37 and 38.

FIG. 41 shows the left-up form, high-distinct target and right-up motion, low-coherence target of the visual motion and visual form attention test of FIGS. 37 and 38.

FIG. 42 displays the left-up form, high-distinct target and right-up motion, high-coherence target of the visual motion and visual form attention test of FIGS. 37 and 38.

FIG. 43 displays the starting phase of a word recognition test battery.

FIG. 44 shows normal letters orientation of the word recognition test battery of FIG. 43.

FIG. 45 shows mirror rotated letters orientation of the word recognition test battery of FIGS. 43 and 44.

FIG. 46 shows inverted letters orientation of the word recognition test battery of FIGS. 43 and 44.

FIG. 47 shows the intermediate phase of the word recognition test battery of FIGS. 43 and 44.

FIG. 48 shows the termination phase of the word recognition test battery of FIGS. 43 and 44.

FIG. 49 illustrates the starting phase of the verbal memory test battery.

FIG. 50 displays the intermediate phase of the verbal memory test battery of FIG. 49.

FIG. 51 illustrates the left-up target orientation with high contrast of the verbal memory test battery of FIGS. 49 and 50.

FIG. 52 shows the right-up target orientation with moderate contrast of the verbal memory test battery of FIGS. 49 and 50.

FIG. 53 displays the right-down target orientation with low contrast of the verbal memory test battery of FIGS. 49 and 50.

FIG. 54 shows a low difficulty facial emotion sensitivity test.

FIG. 55 shows a moderate difficulty facial emotion sensitivity test.

FIG. 56 shows a high difficulty facial emotion sensitivity test.

FIG. 57 shows a low difficulty facial emotion nulling test.

FIG. 58 shows a moderate difficulty facial emotion nulling test.

FIG. 59 shows a high difficulty facial emotion nulling test.

FIG. 60 illustrates a low difficulty social cues sensitivity test.

FIG. 61 illustrates a moderate difficulty social cues sensitivity test.

FIG. 62 illustrates a high difficulty social cues sensitivity test.

FIG. 63 illustrates an exemplary position trace representing target stimulus location (here as x-y display position, ordinates) vs. time in the test (here as z, abscissa).

FIG. 64 illustrates an exemplary speed trace plotting the angular speed of the target (ordinate) vs. time in the test (here as z, abscissa).

FIG. 65 illustrates an exemplary acceleration trace angular acceleration of the target (ordinate) vs. time in the test (here as z, abscissa).

FIG. 66 illustrates an exemplary 3D Signal-to-Noise ratio (S/N or SNR) Gradient plot where the higher points represent high SNR (high perceptual salience, easy to see) and the lower points represent low SNR (low perceptual salience, hard to see).

FIG. 67 illustrates an exemplary S/N profile with respect to vertical and horizontal target positions.

FIG. 68 shows an exemplary position error function profile (the x, y, or angular difference between stimulus target position and subject response cursor position).

FIG. 69 shows an exemplary sampled position error function profile.

FIG. 70 displays an exemplary velocity error function profile.

FIG. 71 is a graphical representation of instantaneous position error.

FIG. 72 is a graphical representation of error magnitude throughout a test (magnitude meaning the absolute value of target-cursor error).

FIG. 73 is a graphical representation of stimulus obscuration over time (high obscuration being harder to see).

FIG. 74 is a graphical representation of subject position error relative to target position over time.

FIG. 75 is a graphical representation of subject velocity error relative to target velocity over time.

FIG. 76 is an exemplary chart illustration summarizing results of automated functional impairment testing displayed in a graphical user interface.

FIG. 77 is a graphical representation showing results of testing for functional impairment over time in an exemplary diagnosis summary.

FIG. 78A shows a display including two concentric annuli in a system for automated impairment assessment testing in which target stimuli in each annulus undergo linked or independent control of obscuration, as the subject rotates a wheel to control target position in one annulus (similar to the cursor in a single annulus stimulus) while the target position is controlled by the algorithm (similar to the target in a single annulus stimulus).

FIG. 78B shows a display including two concentric annuli in a system for automated impairment assessment testing.

FIG. 78C shows a display including two concentric annuli in a system for automated impairment assessment testing.

FIG. 79 is a simplified logic flow diagram showing aspects of a system for automated functional impairment testing.

FIG. 80 is a simplified logic flow diagram showing aspects of a system for automated functional impairment testing.

FIG. 81 is a simplified logic flow diagram showing aspects of a system for automated functional impairment testing.

FIG. 82 is a simplified logic flow diagram showing aspects of a system for automated functional impairment testing.

FIG. 83 is a simplified block diagram showing aspects of a system for automated functional impairment testing.

FIG. 84 is a visual depiction of a series of test scenes in a system for automated functional impairment testing.

FIG. 85 is a visual depiction of a series of test scenes in a system for automated functional impairment testing.

FIG. 86 is a visual depiction of a series of test scenes in a system for automated functional impairment testing.

FIG. 87 is a visual depiction of a series of test scenes in a system for automated functional impairment testing.

FIG. 88 is a visual depiction of a series of test scenes in a system for automated functional impairment testing.

FIG. 89 is a simplified block diagram showing aspects of a system for automated functional impairment testing.

FIG. 90 is a visual depiction of a series of test scenes in a system for automated functional impairment testing.

FIG. 91 is a visual depiction of a series of test scenes in a system for automated functional impairment testing.

FIG. 92 is a visual depiction of a series of test scenes in a system for automated functional impairment testing.

FIG. 93 is a visual depiction of a series of test scenes in a system for automated functional impairment testing.

FIG. 94 is a visual depiction of a series of test scenes in a system for automated functional impairment testing.

FIG. 95 is a simplified block diagram illustrating aspects of a system for automated functional impairment testing and including dual stimulus testing and single stimulus testing alternatively, alternatingly, or singularly deployed based on current or previous test performance, or on subject, group, disease, or treatment criteria.

FIG. 96 is a simplified schematic diagram illustrating aspects of a system for automated functional impairment testing and including dual stimulus testing and single stimulus testing.

FIG. 97 is a simplified block diagram illustrating aspects of a method for automated functional impairment testing and including dual stimulus testing and single stimulus testing.

FIG. 98 is a simplified schematic diagram illustrating aspects of a method for automated functional impairment testing system and including dual stimulus testing and single stimulus testing.

FIG. 99 is a simplified schematic diagram showing aspects of a computing system that may be used in a system for automated functional impairment testing according to an embodiment.

FIGS. 100A, 100B, 100C, 100D, 100E, and 100F detail exemplary visual depictions of a series of test scenes that may be employed by embodiments.

FIGS. 101A, 101B, 101C, 101D, 101E, and 101F detail exemplary visual depictions of a series of test scenes that may be employed by embodiments.

FIG. 102 is a simplified block diagram illustrating aspects of a system for automated functional impairment testing in an embodiment.

FIG. 103 is a simplified block diagram illustrating aspects of a system for automated functional impairment testing in an embodiment.

FIG. 104 is a simplified block diagram illustrating aspects of a system for automated functional impairment testing in an embodiment.

FIG. 105 presents an exemplary heuristic model as may be employed by embodiments of the present disclosure. In this case, dot array contrast sensitivity, presented across background luminance and spatial frequency, is used as an exemplary test. Analogous flow charts might use any other stimulus domain (e.g., shapes, colors, letter, orientations, etc.) might be combined with other tasks (e.g., detection, discrimination, memory, etc.) in the context of other forms of stimulus degradation (e.g., overall luminance, spatial frequency filtering, random dot obscuration, etc.).

FIG. 106 is a schematic diagram representing one embodiment of the Cognivue Test in accordance with an aspect of the present invention.

FIG. 107 is a table compiling cohort data for the Bio-Hermes study utilizing the Cognivue Test.

FIG. 108 is a table showing the biomarker differences between diagnostics groups.

FIG. 109 is a table showing the relationship between the different AD biomarkers that characterize amyloid status.

FIG. 110 is a table showing the strength of association between AD biomarkers and two cognitive screening tests—the Mini-Mental State Examination (“MMSE”) and the Cognivue Test.

FIG. 111 is a graph of specificity versus sensitivity for discriminating between impaired (MCI and AD) and non-impaired. Also included in FIG. 111 is the MMSE, Ray Auditory Verbal Learning Test (“RAVLT”) total score and the Cognivue Test, in accordance with one or more aspects of the present invention.

FIG. 112 is a table showing the area under the curve analysis of the plots shown in FIG. 111.

FIG. 113 is another graph of specificity versus sensitivity for discriminating healthy controls from MCI, in accordance with one or more aspects of the present invention.

FIG. 114 is a table showing the area under the curve analysis of the plots shown in FIG. 113.

FIG. 115 is a block diagram for the breakdown of the population of people participating in the Bio-Hermes study, in accordance with one or more aspects of the present invention.

FIG. 116 is a table summarizing the differences between Cognivue Test scores and cognitive/biomarker status.

FIG. 117 is a table summarizing the differences between the twelve individual Cognivue Test subtests and clinical/biomarker status.

FIG. 118 is a table summarizing the calculated area under the curve results for ROC curves discriminating between True Controls from Preclinical AD.

FIGS. 119A-119D show histograms of each of the Cognivue Amyloid Risk Measure (CARM) components separated by amyloid positivity based on SUVR threshold.

FIG. 120 is a table showing the distribution and evaluation of statistical differences using pairwise t-tests.

FIG. 121 is a table summarizing the performance of the different models using the CARM component scores.

FIG. 122 is a table showing CARM results based upon a 3-point scale.

FIG. 123 is a table showing independent sample ANOVAs (Analysis of Variance) with Bonferroni-corrected pairwise post hoc tests for the results tabulated in FIG. 122.

FIG. 124 is a table showing CARM results based upon a 5-point scale.

FIG. 125 is a table showing independent sample ANOVAs (with Bonferroni-corrected pairwise post hoc tests for the results tabulated in FIG. 124.

Corresponding reference characters indicate corresponding parts throughout the several views. The embodiments set out herein are examples only and illustrate currently preferred embodiments of the present invention, and such examples are not to be construed as limiting the scope of the invention in any manner.

### DETAILED DESCRIPTION OF THE INVENTION

The present disclosure is related to the subject matter disclosed in the following co-pending application filed on Sep. 16, 2009 and each naming Charles Joseph Duffy as the inventor: Ser. No. 12/560,583 and entitled METHOD AND SYSTEM FOR QUANTITATIVE ASSESSMENT OF FUNCTIONAL IMPAIRMENT.

In describing embodiments of the present invention as illustrated in the drawings, specific terminology may be employed for the sake of clarity. In the present specification, an embodiment showing a singular component should not be considered limiting. Rather, the subject matter encompasses other embodiments including a plurality of the same component, and vice-versa, unless explicitly stated otherwise herein. Moreover, applicant does not intend for any term in the specification or claims to be ascribed an uncommon or special meaning unless explicitly set forth as such. Further, the present subject matter encompasses present and future known equivalents to the known components referred to herein by way of illustration.

Additional context regarding the field of this disclosed subject matter is provided by the following patents, all of which have common assignment and invented by Charles Joseph Duffy, and all of which are incorporated by reference in their entirety for all purposes into this detailed description: U.S. application Ser. No. 10/703,101, entitled “Method for Assessing Navigational Capacity”, Duffy et al.; U.S. Pat. No. 6,364,845B1, entitled “Methods for Diagnosing Visuospatial Disorientation Or Assessing Visuospatial Orientation Capacity”, Duffy et al.

Further information regarding the field of this disclosed subject matter appears in the following research publications, all of which have common authorship by Charles Joseph Duffy and all of which are incorporated by reference in their entirety for all purposes into this detailed description: Duffy, Charles J. et al., “Attentional Dynamics and Visual Perception: Mechanisms of Spatial Disorientation In Alzheimer's Disease”, Brain, 126:1173-1181 (2003); Duffy, Charles J. et al., “Visual Mechanisms of Spatial Disorientation in Alzheimer's Disease”, Cerebral Cortex, 11:1083-1192 (2001).

It should be understood that wherein this disclosure refers to specific diagnostic techniques, such diagnostic techniques may be performed by operations of a diagnostic computing system specifically implemented on, and calibrated for, desktop, laptop, mobile, or network hardware computer devices in communication with a suitable manual, ocular, or physiological input device and display. In embodiments, a suitable input device may be calibrated to provide known, predetermined responsiveness to input of a processed output, such as a pointer or cursor, that may be displayed for a user to manipulate or control the movement of such a pointer or cursor on or relative to a sensory stimulus field of display. It will be understood that in embodiments response of processed output such as a cursor or pointer to manual input may be received in relation to an input device in a high precision relationship.

In the present disclosure, the phrase “optic flow” may be defined as the patterned visual motion seen by a moving observer, or simulating what is seen by a moving observer, that provides clues about heading direction and the three dimensional structure of the visual environment (Duffy et al., “Visual Mechanisms of Spatial Disorientation in Alzheimer's Disease”). Impaired optic flow processing is debilitating, for example, as it relates to individual autonomy of ambulatory or vehicular self-movement perception and control.

One direct example from the inventor's published research related to how impaired optic flow perception may include, but are not limited to, elementary visual motion processing deficits and elevated perceptual thresholds. Advantages of the present disclosure can be derived from essentially any analysis of the impaired higher-order (complex stimulus) recognition which may be rooted in elementary brain processing impairments (e.g., optic flow), and how it relates to the perceptual mechanisms of complex behavior (e.g., visuospatial orientation) that reflects the impaired appreciation and control of behavior considering the relations between the observer and features of the environment including earth-fixed objects and independently moving objects, persons, or vehicles.

The present disclosure describes systems, methods, and computer implemented code in a suitable accessible memory, for diagnosis of a patient. Specifically, the present disclosure describes systems, methods, and computer-implemented code in suitable accessible memory, for diagnosis of a patient including or utilizing a dual input mechanism. Advantages of disclosed systems, methods, and computer implemented code over previous diagnosis techniques include but are not limited to:
i. the use of objective neural input systems including single or multiple sensory stimulus arrays (e.g. size and/or color and/or expression facial discrimination),
ii. the use proscribed behavioral, cognitive, and emotional tasks that engage the test subject in specific information processing paradigms (e.g., manual pointing or gaze shifting to the most asymmetrically shaped object tree in the array of trees),
iii. the use of objective behavioral or physiological response monitoring systems for assessing and inter-relating stimulus and task related effects reflecting neural information processing (e.g., heart-rate changes and speed of response to manually move a cursor to the most threatening face),
iv. the random setting of specific stimulus examples and motor response requirements to create a diverse set of conditions within test categories and parameters so that each running of all tests for all subjects may be unique (e.g., a different set of words and non-words is presented in every stimulus of a word discrimination test presented to each subject on each occasion),
v. the cross-calibration of a series of tests, within and between test sessions, to standardize stimulus and response parameters relative to the specific attributes individual subject (e.g., handicapping for hand movement slowing in assessing manual response speed to the most unique stimulus in an array),
vi. the use of heuristic algorithms to select the next most informative test to be administered to a test subject based on that subject's performance on previous tests in that test session, or in previous test sessions, or based-on established or putative diagnoses, or based-on the administration of therapeutic or response-provocative agents (e.g.),
vii. the consistency of tests achieved by the elimination by operator/administrator control or influence over the pace, content and conduct of each test and of the sequence of tests to assure complete consistency of those categorical and parametric variables specifying the details of the tests.

Embodiments of the present disclosure may employ visual salience testing, of which contrast testing for example, may be a component, to determine threshold competencies. For example, a contrast sensitivity test may include manipulating the contrast of one or more dots and observing results across the contrast range. Some embodiments may likewise perform luminance and spatial frequency testing.

Embodiments of the present disclosure may perform additional tests, including but not limited to: perception and memory tests, in determining diagnosis reports. In some tests, of random clutter, noise, and other methods may be employed to modify the presented signal to noise ratio. to change the SNR. Using the language of contrast sensitivity testing greatly distracts from and diminishes the intellectual property. 3) Please see the manuals for descriptions of some ways in which the SNR is being manipulated.

A simplistic representation of a test employed by embodiments may be include:
i. a) presenting a stimulus domain, wherein exemplary stimulus domain class include but are not limited to, letters, shapes, motion, spatial arrangement, faces, and hands;
ii. b) modifying a stimulus parameter to vary the signal to noise ratio, wherein this may be subject to, or dependent upon, prior test performance; and
iii. c) requesting input responsive to a particular task, which creates the behavioral response paradigm. For example, particular tasks may include but are not limited to detection, memory, and attention tasks.

A further simplistic representation of an exemplary test employed by embodiments may be considered to include:
i. a stimulus domain;
ii an SNR control parameter; and
iii. a task.

A simplistic representation of some embodiments of the present disclosure, includes a test scenario of:
i. performing a simple motor control test, the results of which may be used to set the pace and scoring for subsequent tests.
ii. performing a visual salience test, the results of which may be used set the contrast and size of the other tests.

Some embodiments may employ these derived parameters to one or more subsequent tests. E.g. If someone is slow, slow it down; and if someone has poor vision, make it easier to see. In some embodiments, subsequent tests may maintain those derived parameters without manipulated (same subject in the same session, those parameters are fixed by the results of those first tests). An exemplary test, independent of the derived values, may include a stimulus SNR control parameter (% random, angle changes, etc), which may be used in that particular test to manipulate the SNR (task difficulty).

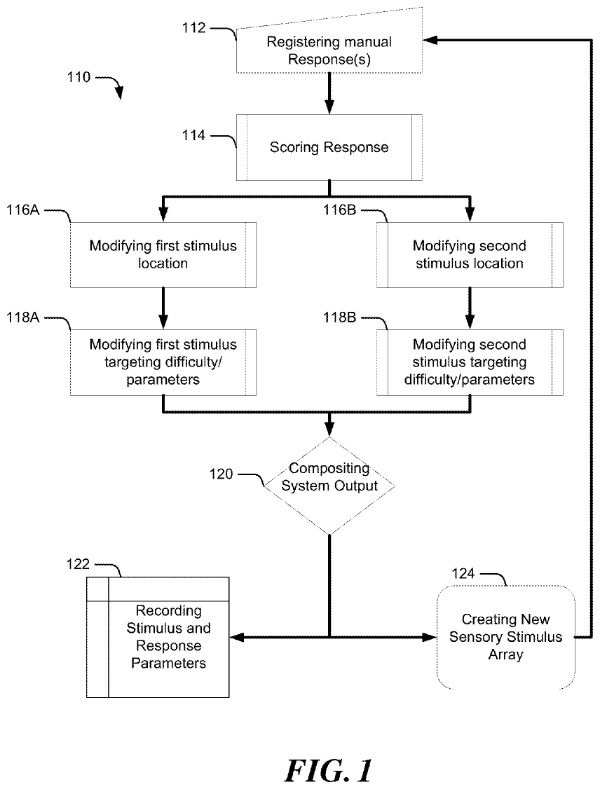

FIG. 1 shows a conceptual framework of the interacting subsystems 110 in the environment that is used to assess functional impairment in a subject. As shown, an exemplary process may commence by the registering of the user's input 112, wherein the system scores the inputs 114. The system may then modify the first stimulus location 116A, and may modify the first stimulus targeting difficult or presentation parameters 118A. The system may coincidently, or at a pre-defined delay, modify the second stimulus location 116B, and may modify the second stimulus targeting difficult or presentation parameters 118B. Thereafter, system may composite the system output 120, record the stimulus response parameters 122, and/or may create a new sensory stimulus array 124, wherein the user's inputs are again registered 112.

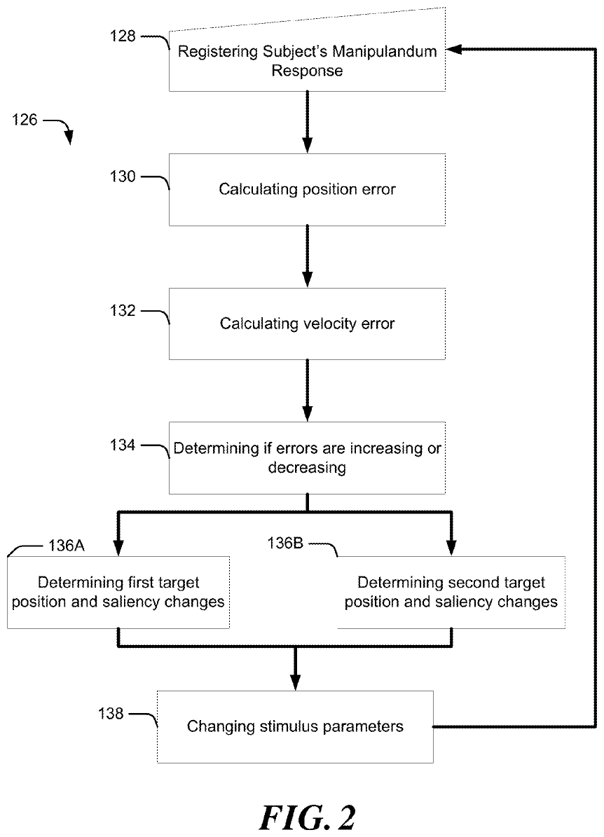

FIG. 2 displays an exemplary workflow method for assessing functional impairment of a patient or subject. The functional impairment 126 workflow commences at step of register subject's manipulandum response 128. Immediately thereafter is the step of calculate position error 130, which is followed by the step of calculate velocity error 132. After step 132, the step of determine if errors are increasing or decreasing 134 occurs, which may be followed coincidently, or at a defined delay, with the step of determining the first target position and saliency changes 136A and the first target position and saliency changes 136B. Immediately thereafter, the step of change to new stimulus parameter 138 occurs; thereafter, is the step of step of register subject's manipulandum response 128, which results in repeating the ensuing steps of the workflow of functional impairment 126.

As appearing in the present disclosure, sensory salience relates to the perceptibility of a stimulus as judged by the observer's ability to respond to that stimulus, or for a person or device to detect some change in the observer, based on the presentation of that stimulus. Salience can be affected by any categorical or parametric change in the physical properties of the stimulus. These properties include, but are not limited to, changes in luminance, contrast, stimulus degradation, etc.

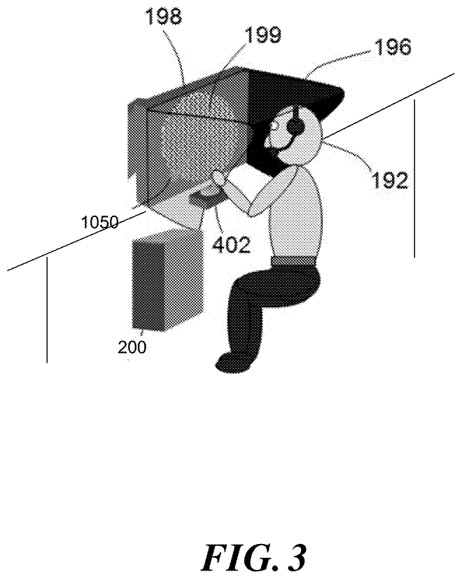

FIG. 3 depicts a test environment 188 that may be associated with quantitative assessment of functional impairment. The test environment 188 may include, but is not limited to those associated with research and development laboratories, such as those present at medical centers, universities, drug companies, and pharmaceutical companies. Further, quantitative assessment of functional impairment may be conducted in clinics as well as animal research facilities. The present subject matter may be implemented in future known equivalents.

Further, quantitative assessment of functional impairment may be conducted remotely from any physical location via the Internet or other network. In addition, the present disclosure may be utilized for performing therapy, screening tests or more formal evaluations over the Internet.

The present disclosure may provide a test environment 188, which may include a versatile psychophysical testing environment that simplifies complex experimental paradigms. The present disclosure may assist clinicians and/or researchers with replicating fundamental studies and better investigating visual functions that are impaired by aging and neural dysfunctions, such as tone and synchrony of acoustic stimuli and shape and motion of visual stimuli.

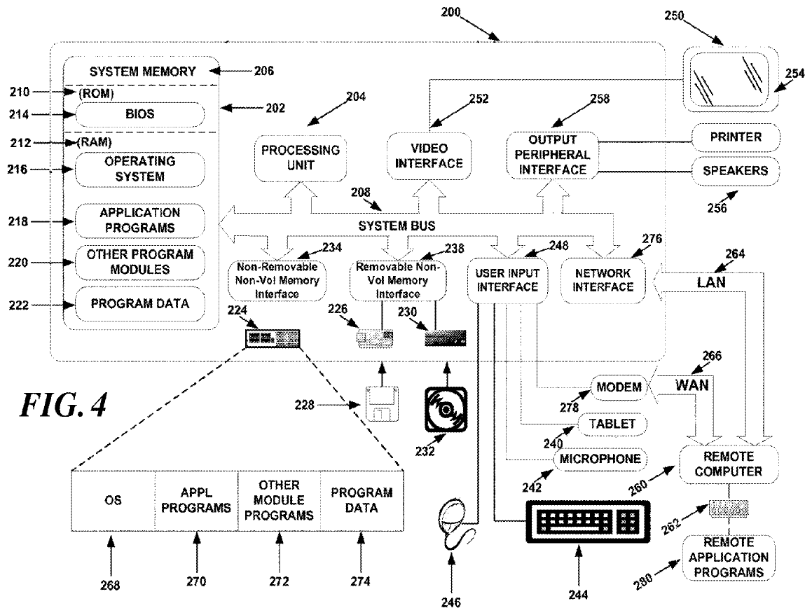

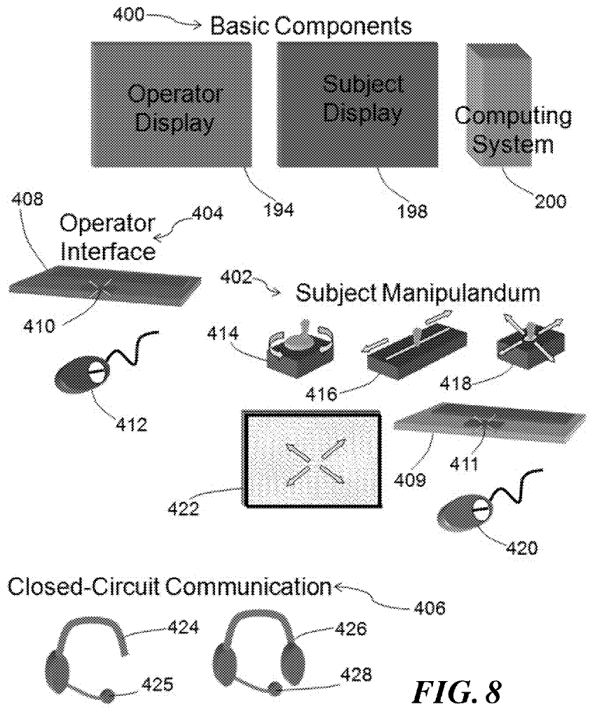

Further, the exemplary test environment 188, which is depicted in FIG. 3, may include a mounted shroud-box enclosure that may shield the subject 192 from visual distractors. In systems designed for quantitative assessment of functional impairment, a variety of component and devices comprise the necessary equipment. The test environment 188 in the present disclosure may include, but is not limited to, a subject 192, operator 190, subject display 198, stimulus area 199, operator display 194, a subject manipulandum 402, a shroud 196, a subject earphones and a subject microphone, an operator earphones and an operator microphone, and a computing system 200. Further, the subject headset 426, which may include a subject earphones and a subject microphone, is shown in greater detail in FIG. 8. Further, the operator headset 424, which may include an operator earphones and an operator microphone, is shown in greater detail in FIG. 8. More particularly, the computing system 200 is shown in greater detail in FIG. 4.

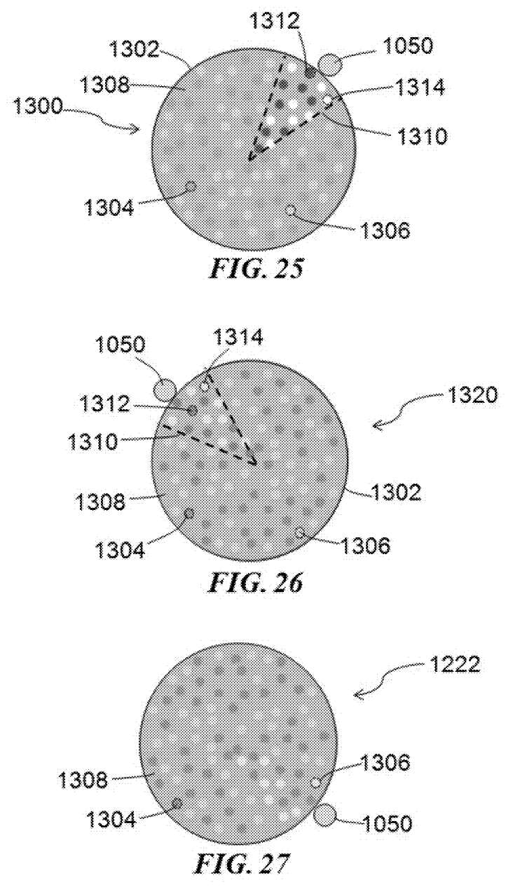

The stimulus area may be presented on the subject display 198 and/or the subject speakers/earphones, wherein the subject earphones may be a component of subject headset or of the surrounding test apparatus 426. Further, the cursor 1050 may be located on the subject display 198. The cursor 1050 may extend from the center of the stimulus area 199 to the edge of a stimulus area 199, such as a circular border 1302, which is shown in greater detail in FIG. 25.

Further, the cursor 1050 may be the same cursor that is implemented in multiple tests of the present disclosure, with the exception of superimposed tests. More particularly, functional impairment tests that include superimposed phenomena, may require the alignment of one target area with another target area, thereby requiring more than one cursor 1050.

Further, the test environment 188 may include a mount device, which may be a pull-mount or a desk-mount. Further, the subject display 198 may include, but is not limited to, a display screen, wireless connection, etc. The auditory stimulator (e.g., speakers of headset) or the visual display (e.g., screen or goggles) may be used to display instructions, to display an image of the operator 190 during instructions or coaching, or to present the visual test stimuli. The display device 22 may include, or could have attached, a video camera directed at the subject 192 to show an image of the subject 192 on operator display 194. The subject display 198, which is that of the subject 192, may include a shroud 196 mounted onto a box, in the form of a shroud-mounted box, in order to shield the subject 192 from the visual distractors, or may also include earphones in order to present stimuli and shield the subject from audible distractors.

With reference to FIG. 4, an exemplary system within a computing environment for implementing the invention includes a general purpose computing device in the form of a computing system 200, commercially available from Intel, IBM, AMD, Motorola, Cyrix and others. Components of the computing system 202 may include, but are not limited to, a processing unit 204, a system memory 206, and a system bus 236 that couples various system components including the system memory to the processing unit 204. The system bus 236 may be any of several types of bus structures including a memory bus or memory controller, a peripheral bus, and a local bus using any of a variety of bus architectures.

Computing system 200 typically includes a variety of computer readable media. Computer readable media can be any available media that can be accessed by the computing system 200 and includes both volatile and nonvolatile media, and removable and non-removable media. By way of example, and not limitation, computer readable media may comprise computer storage media and communication media. Computer storage media includes volatile and nonvolatile, removable and non-removable media implemented in any method or technology for storage of information such as computer readable instructions, data structures, program modules, cloud storage, or other data storage apparatus.

Computer memory includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by direct or transmitted connection to the fixed or mobile computing system 200.

The system memory 206 includes computer storage media in the form of volatile and/or nonvolatile memory such as read only memory (ROM) 210 and random access memory (RAM) 212. A basic input/output system 214 (BIOS), containing the basic routines that help to transfer information between elements within computing system 200, such as during start-up, is typically stored in ROM 210. RAM 212 typically contains data and/or program modules that are immediately accessible to and/or presently being operated on by processing unit 204. By way of example, and not limitation, an operating system 216, application programs 220, other program modules 220 and program data 222 are shown.

Computing system 200 may also include other removable/non-removable, volatile/nonvolatile computer storage media. By way of example only, a hard disk drive 224 that reads from or writes to non-removable, nonvolatile magnetic media, a magnetic disk drive 226 that reads from or writes to a removable, nonvolatile magnetic disk 228, and an optical disk drive 230 that reads from or writes to a removable, nonvolatile optical disk 232 such as a CD ROM or other optical media could be employed to store the invention of the present embodiment. Other removable/non-removable, volatile/nonvolatile computer storage media directly connected, or accessed by transmission-based connectivity, locally or remotely, that can be used in the exemplary operating environment include, but are not limited to, magnetic tape cassettes, flash memory cards, digital versatile disks, digital video tape, solid state RAM, solid state ROM, and the like. The hard disk drive 224 is typically connected to the system bus 236 through a non-removable memory interface such as interface 234, and magnetic disk drive 226 and optical disk drive 230 are typically connected to the system bus 236 by a removable memory interface, such as interface 238.

The drives and their associated computer storage media, discussed above, provide storage of computer readable instructions, data structures, program modules and other data for the computing system 200. For example, hard disk drive 224 is illustrated as storing operating system 268, application programs 270, other program modules 272 and program data 274. Note that these components can either be the same as or different from operating system 216, application programs 220, other program modules 220, and program data 222. Operating system 268, application programs 270, other program modules 272, and program data 274 are given different numbers hereto illustrates that, at a minimum, they are different copies.

A user may enter commands and information into the computing system 200 through input devices such as a tablet, or electronic digitizer, 240, a microphone 242, a keyboard 244, and pointing device 246, commonly referred to as a mouse, trackball, or touch pad. These and other input devices are often connected to the processing unit 204 through a user input interface 248 that is coupled to the system bus 208, but may be connected by other interface and bus structures, such as a parallel port, game port or a universal serial bus (USB).

A monitor 250 or other type of display device is also connected to the system bus 208 via an interface, such as a video interface 252. The monitor 250 may also be integrated with a touch-screen panel or the like. Note that the monitor 250 and/or touch screen panel can be physically coupled to a housing in which the computing system 200 is incorporated, such as in a tablet-type personal computer or other mobile computer linked device. In addition, computers such as the computing system 200 may also include other peripheral output devices such as speakers 254 and a computer linked printer 256, which may be connected through an output peripheral interface 258 or the like.

Computing system 200 may operate in a networked environment using logical connections to one or more remote computers, such as a remote computing system 260. The remote computing system 260 may be a personal computer, a server, a router, a network PC, a peer device, a personal mobile device, or other common network node, and typically includes many or all of the elements described above relative to the computing system 200, although only a memory storage device 262 has been illustrated. The logical connections depicted include a local area network (LAN) 264 connecting through network interface 276 and a wide area network (WAN) 266 connecting via modem 278, but may also include other networks such as transmission accessed storage media or processing devices. Such networking environments are commonplace in offices, enterprise-wide computer networks, intranets, the Internet, and cloud systems.

For example, in the present embodiment, the computer system 200 may comprise the source machine from which data is being generated/transmitted, and the remote computing system 260 may comprise the destination machine. Note however that source and destination machines need not be connected by a network or any other means, but instead, data may be transferred via any media capable of being written by the source platform and read by the destination platform or platforms.

The central processor operating pursuant to operating system software such as IBM OS/2®, Linux®, UNIX®, Microsoft Windows®, Apple Mac OSX® and other commercially available operating systems provides functionality for the services provided by the present invention. The operating system or systems may reside at a central location or distributed locations (i.e., mirrored or standalone).

Software programs or modules instruct the operating systems to perform tasks such as, but not limited to, facilitating client requests, system maintenance, security, data storage, data backup, data mining, document/report generation and algorithms. The provided functionality may be embodied directly in hardware, in a software module executed by a processor or in any combination of the two.

Furthermore, software operations may be executed, in part or wholly, by one or more servers or a client's system, via hardware, software module or any combination of the two. A software module (program or executable) may reside in RAM memory, flash memory, ROM memory, EPROM memory, EEPROM memory, registers, hard disk, a removable disk, a CD-ROM, DVD, optical disk or any other form of storage medium known in the art. An exemplary storage medium is coupled to the processor such that the processor can read information from, and write information to, the storage medium. In the alternative, the storage medium may be integral to the processor. The processor and the storage medium may also reside in an application specific integrated circuit (ASIC). The bus may be an optical or conventional bus operating pursuant to various protocols that are well known in the art.

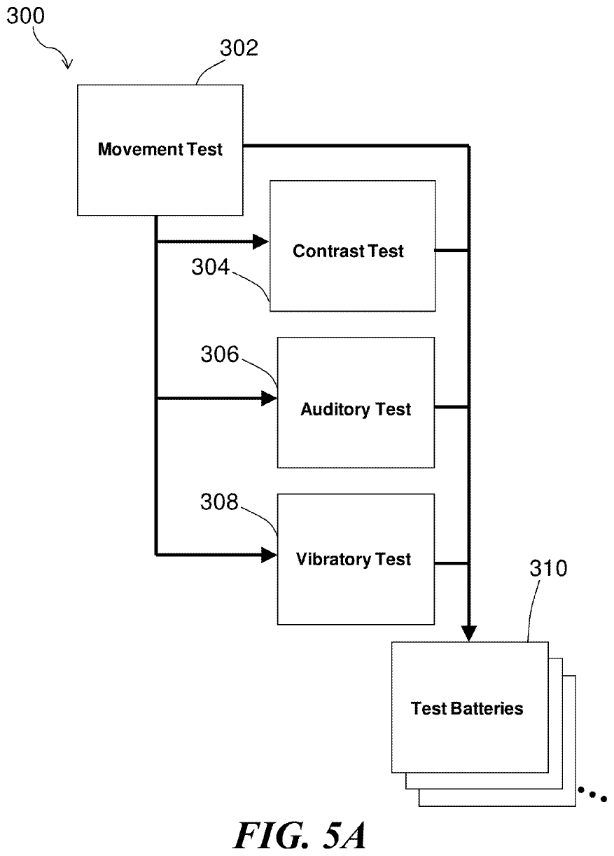

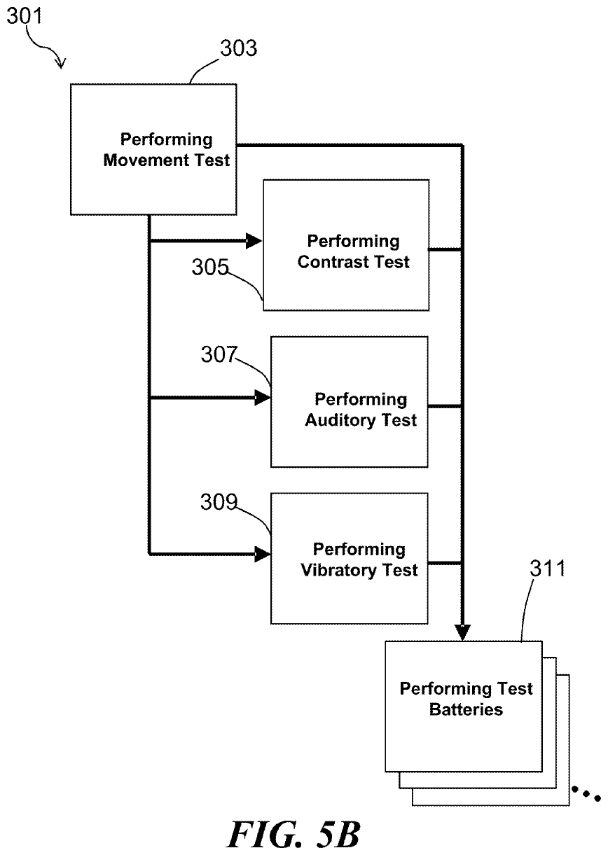

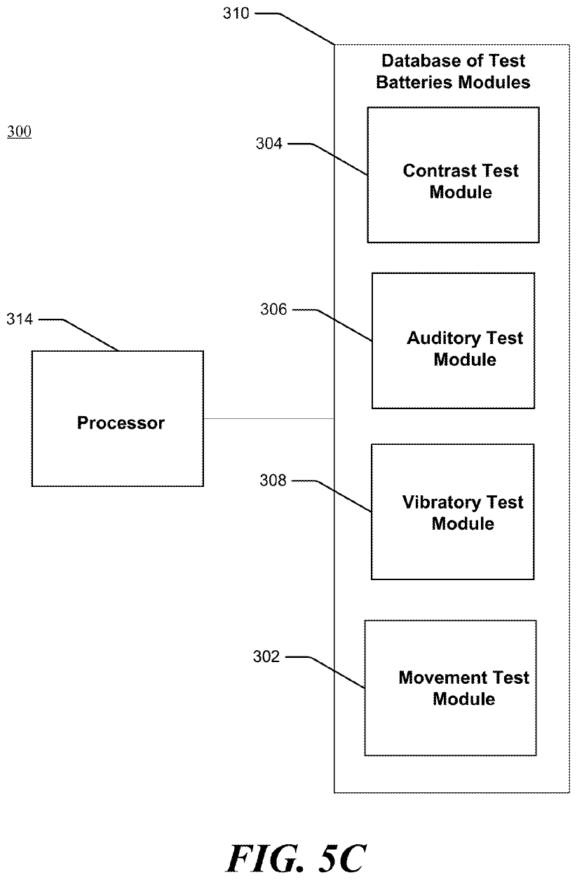

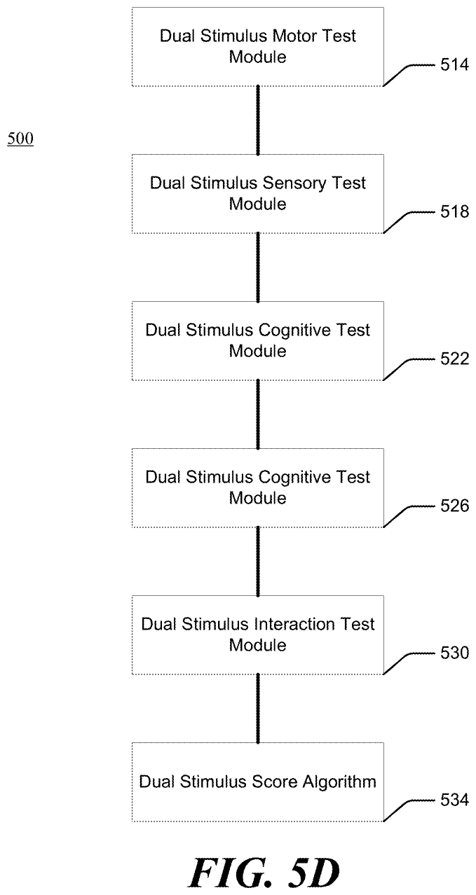

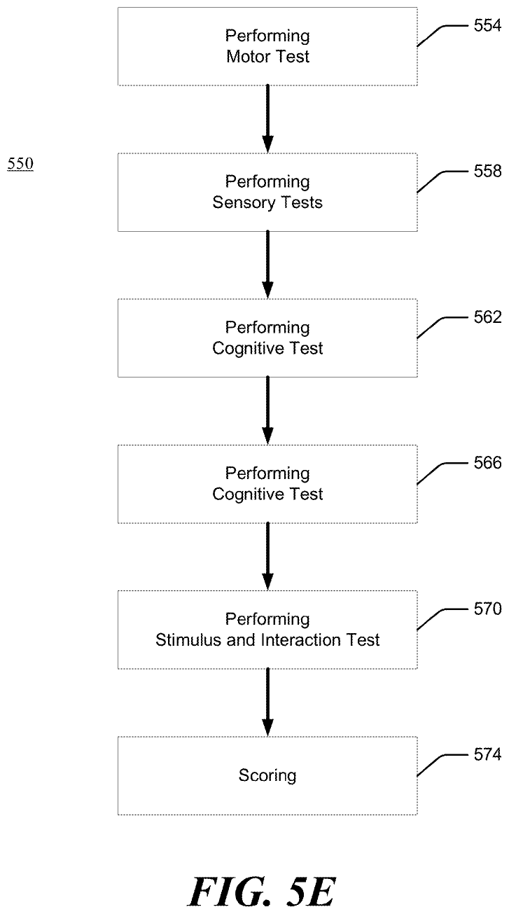

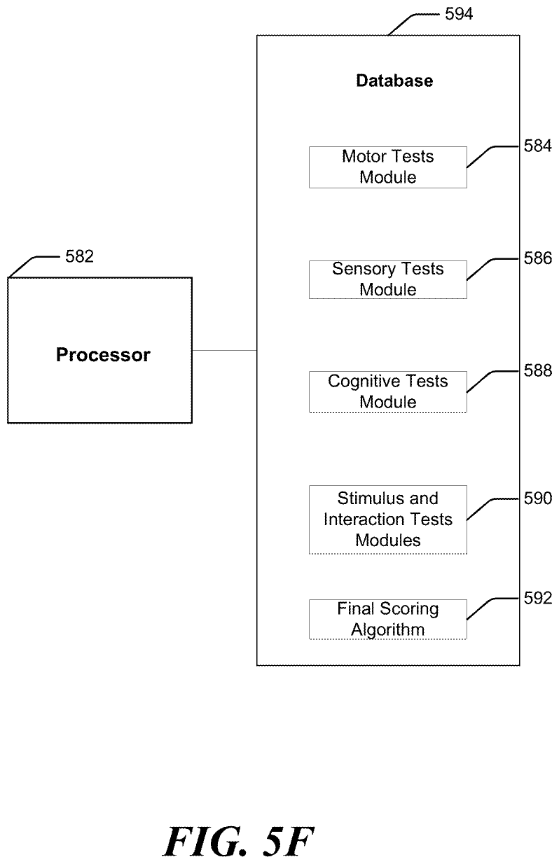

FIG. 5A shows the paradigm of a hierarchical nature of parametric individualization. The word “hierarchical” refers to some tests that may derive measures that may be used as pre-sets for subsequent tests, or for heuristically selected subsequent tests drawn from a fixed-set or variably applied subsequent tests. Further, the word, “hierarchical” is associated with the occurrence of start values in subsequent tests, such that there may be an ordered sequence of tests. In the hierarchy for parametric individualization 300, the resulting data from a motor adaptation test 302 may be applied to a visual saliency test 304, an auditory test 306, and/or a vibratory test 308. In some embodiments, results may also be applied to perception tests, and/or memory tests, as detailed herein. The results of the one particular test or a combination of tests that may include, but are not limited to, a visual saliency test 304, an auditory test 306, and/or a vibratory test 308, may be applied to the test batteries 310, which are further described in the present disclosure.

FIG. 5B shows the paradigm of a hierarchical nature of parametric individualization. As shown, a system employing the exemplary hierarchy for parametric individualization 300, may perform a movement test 303, wherein the results of the movement test may be applied to a visual saliency test 305, an auditory test 307, a vibratory test 309, or combinations thereof.

FIG. 5C illustrates an exemplary system and the soring of a visual saliency test module 304, an auditory test module 306, a vibratory test module 308, and a movement test module 302, that may be actuated by system processor 314.

FIG. 5D presents an exemplary process flow, as may be employed by system embodiment of the present disclosure. As shown, process flow 500, may include running a dual stimulus motor test module 514, which may be followed by running a dual stimulus sensory test module 518, which may be followed by running a dual stimulus cognitive test module 526, which may be followed by running a dual stimulus interaction test module 530, which may be followed by running a dual stimulus scoring algorithm 534.

FIG. 5E presents an exemplary method as employed by embodiments of the present disclosure. As shown, method 550, may include performing a motor test 554, which may be followed by performing a sensory test 558, which may be followed by performing a cognitive test module 562, which may be followed by performing a cognitive test module 566, which may be followed by performing a interaction test 570, which may be followed by results scoring 574.

FIG. 5F presents an exemplary system architecture, including storage of modules within a database 594. As shown, database 594, may include a motor test module 584, sensory test module 586, cognitive test module 588, stimulus and interaction test modules 590, and final scoring algorithms 592.

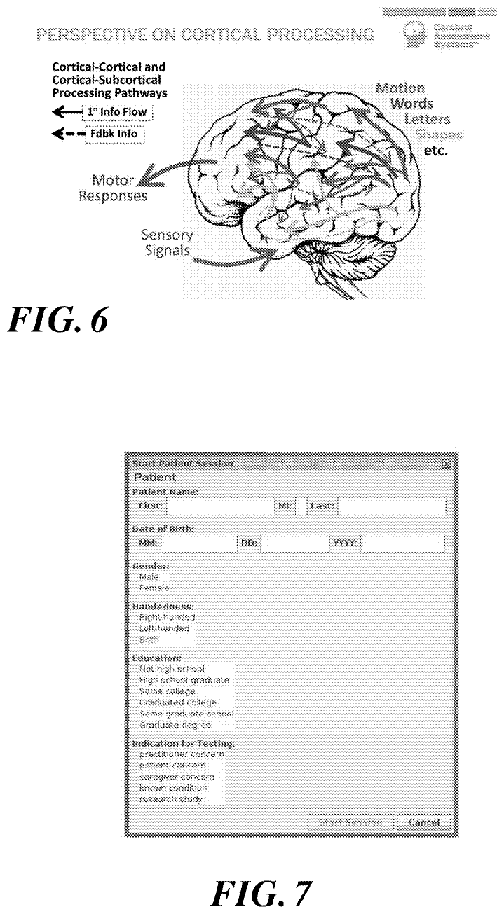

FIG. 6 portrays a representation of left posterior-lateral view 320 of the human brain 322. The example given is of the human visual system organized, as are other cortical sensory systems, as a series of parallel information processing pathways. In the eyes, there are two sensory system, cone cells for daylight vision and rod cells for twilight vision. In the optic nerves and visual pathways, there are several different types of nerve fibers, of which the magnocellular pathway 324 and the parvocellular pathway 328 are the most important. The magnocellular pathway 324 is considered by those skilled in the art to be the “where?” pathway; the parvocellular pathway 328 is considered by those skilled in the art to be the “what?” pathway. Further, the magnocellular pathway 324 carries all transient, motion related visual information and low contrast black and white information. The parvocellular pathway 328 carries all color information and is effective in carrying high contrast black and white information. Further, the human brain 322 includes a striate and peri-striate visual areas 326.

FIG. 7 display an exemplary operator display 194, which may display to an operator 190 perceived impairment data for a subject 192. The operator display 194 may include, but is not limited to, a real-time subject video display 332, a stimulus display 334, a current test performance display 336, and a subject error display 338. Further, the operator display 194 may display the current status 362, which may include, but is not limited to, the current status of the current subject, the current status of the current test, and the current status of the current scores. Further, the test performance display 336 may show a graph of stimulus difficulty 350 versus the time of time intervals 348.

The system may be configured to allow an operator 190 to select the appropriate test from test batteries 310 via the option of select and store test batteries 340. Alternatively, the system may recommend an appropriate test based on historical patient data or other inputs with test selection heuristics based-on immediately, or remotely, previously administered performance tests, or other subject characteristics, or the characteristics of specific circumstances of interest related to that subject, as in suspected disorders or brain function or expected circumstances or high performance in particular areas. The operator display 194 shared by or separate from the linked to the subject display facilitates input commands, for example start 342, pause 344, and stop 346 with respect to any functional assessment test. A functional assessment test may be symbolized as test battery A 352, test battery B 354, test battery C 356, test battery D 358, or test battery X 360, as in shown on the exemplary operator display 194 of FIG. 7.

Further, the operator display 194 may be used to start and stop testing via a series of windows that may be shown by the use of the computing system 200. The series of windows may include the following:
iv. A window for data entry regarding the subject 192, operator 190, and test site.
v. A window for the operator 190 being able to view the subject's stimulus for monitoring.
vi. A window for the display of the current subject 192 and ongoing test.
vii. A window for the real-time display of graphical subject error and numerical subject error.
viii. A window for the display of the subject's video image to the operator 190 for the monitoring of the subject's position and gaze.
ix. A window for the display of the subject's response saliency function.
x. A window for the display of the subject's current basic scores.
xi. A window for the operator 190 to enter comments.
xii. A window for the operator 190 to enter identifying, medical history, treatment, etc.

The operator display 194 may be one component, of many components, that may be utilized for quantitative assessment of functional impairment. FIG. 8 illustrates an embodiment of the principal components of the presently disclosed method for assessment of functional impairment. The components may include, but are not limited to, basic components 400, a subject manipulandum 402, an operator interface 404, and closed-circuit communication 406. The basic components 400 may be utilized in the test environment 188, as is shown in FIG. 3.

The operator interface 404, may include, but is not limited to devices specifically for use by the operator 190, such as a keyboard 244, herein called operator keyboard 408, and a pointing device 246, which may be, but is not limited to, an operator touchpad 410 or a mouse, herein called an operator mouse 412. An operator 190 may enter commands and information into the computing system 200 through input devices such as an operator touchpad 410 or an operator mouse 412. The operator 190 may utilize the operator interface 404 for entering identifying information, medical history, treatment data, etc. to facilitate in quantitative assessment of functional impairment.

Further, the closed-circuit communication 406 may include, but is not limited to, an operator headset 424, which may be utilized by the operator 190, and a subject headset 426, which may be utilized by the subject 192. The present disclosure may include a closed-circuit auditory and visual links 406 between the subject 192 and a human or simulated operator, on-site or linked from a remote location, 190 that consists of three components:
i. The subject 192 may utilize a subject headset 426 to shield from audible distractors, thereby allowing for the controlled presentation of auditory stimuli as task cues or distractors, or cue elements of the task, which include, but are not limited to, specific tones and words, or for instructions or for coaching by the operator 190. The subject headset 426 may include a co-mounted subject microphone 428, which may always be on to the operator 190, thereby allowing all comments by the subject 192 and eliciting appropriate responses.
xiii. The operator 190 may wear an operator headset 424 that may allow the operator 190 to hear any sounds from the subject 192 but also may allow the operator 190 to hear sounds from the surrounding environment. The operator headset 424 may include a co-mounted operator microphone 425, which may allow the operator 190 to speak with the subject 192. Further, the operator interface 404 may allow for contact with the subject 192 via the operator 190 being able to enable or disable a virtual switch in the operator display 194.
xiv. The present disclosure includes software, hardware, and interface connections for controlling the state of the subject-operator closed-circuit communication 406.

Further components of the present disclosure may include a subject manipulandum 402, which may be a physical interfacing device that transforms input from a user. The properties of the subject manipulandum 402 may be akin to the properties of a pointing device 246 or other input devices, which may include, but is not limited to a wheel, a joystick, or a computer mouse device. Further, the subject manipulandum 402 may be a touch screen display panel, a movement, tilt, contact, or pressure sensitive device, or a device that by remote sensing monitors subject movements of hands, eyes, head, or other body parts, or speech or automatic body responses and 422 that can accommodate continuous or intermittent input.

Similar to the operator interface 404, the subject manipulandum 402 may include, but is not limited to devices, such as a keyboard 244, herein called subject keyboard 409, and a pointing device 246, which may be, but is not limited to, a subject touchpad 411 or a mouse, herein called an subject mouse 420 also including a movement, tilt, contact, or pressure sensitive device, or a device that by remote sensing monitors subject movements of hands, eyes, head, or other body parts, or speech or automatic body responses. A subject 192 may enter commands and information into the computing system 200 through input devices such as a subject touchpad 411 or mouse 420 or other direct or remote contact device.

Further, the system may be configured for exclusive subject input by directly or remotely responding as illustrated here by moving the position of the subject response manipulandum 402. The subject manipulandum 402 may be manipulated by the hand or other volitional movement, or non-volitional response of the subject 192, and its purpose is to maximize stimulus response compatibility so the sensory processing and motor control aspects of brain function being engaged by the stimuli presented and the task engaging the subject. The subject 192 may provide input and respond to sensory stimuli by movement of the subject manipulandum 402. Exemplary arrangements of the subject response devices are hand contact manipulandums, including rotary manipulandums 414, linear manipulandums 416, and xy Cartesian manipulandums 418. Thus, the subject response manipulandum 402 may register single or multi-axis responses such as moving in rotation motion 440, a linear motion 442, x-axis motion in the Cartesian coordinate system 444, y-axis motion in the Cartesian coordinate system 446, and combinations thereof. In addition, the movement of the subject response manipulandum 402 may be represented as a cursor 1050 on the subject display 198. The cursor may be, but is not limited to, a ball-and-stick cursor.

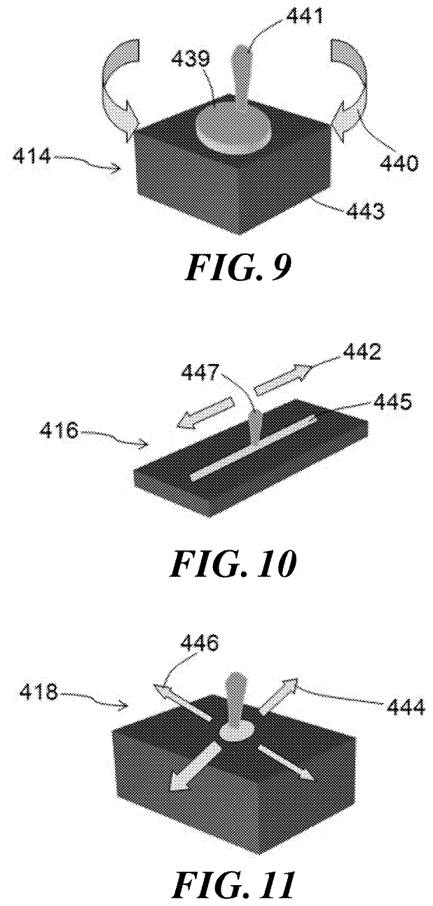

With reference to FIGS. 9, 10, and 11, an exemplary a rotary manipulandum 414, an exemplary linear manipulandum 416, and an exemplary xy Cartesian manipulandum 418 are shown in greater detail but do not set these examples apart from other subject response interface devices contacting, or remotely sensing, of volitional movements such as eye, head, and body movement or monitoring other body responses such as heart rate, respiratory rate or brain electrical responses measured by integral or attached machines and evoked by the presented stimuli and tasks.

Further, the subject manipulandum 402 may be designed to incorporate a means of monitoring whether the subject 192 is contacting a handle through a capacitive contact detector. Further, the subject manipulandum 402 may be designed to incorporate a motorized system that can alter the resistance offered by the subject manipulandum 402 to the subject 192 by moving it for use in testing the motoric control of the subject 192. Further, the subject manipulandum 402 may be designed to incorporate a vibrating element that can create a variable amplitude, variable frequency vibration of a handle as a cue or a distracting stimulus.

Further, the present disclosure may accommodate the use of a plurality of subject response devices, here again exemplified by the subject response manipulandum 402 to test the motoric control of the subject 192. The present disclosure may accommodate two manipulanda 402, one with each of the subject's hands.

Further, the response of the subject manipulandum 402 may be implemented as separate box mounted devices or virtual devices on a touch screen display panel 422 that can accommodate finger or stylus input, such as by text.

Further principal components of a computing system 200, may include a computer readable medium, a computing process, that supports detailed operations by interfacing with other hardware components and by representative software described in the further in the present disclosure.

In the illustrated example shown in FIG. 9, the subject manipulandum 402 is shown as a rotary manipulandum 414 that moves in a rotational motion 440. The rotary manipulandum 414 may consists of a box mounted wheel 439, which may be mounted such that it can rotate around its center, which may be attached to a rotation circuit in the box 443. The box mounted wheel 439 is moved by grasping an eccentric handle 441 that the subject 192 uses to rotate the angle of the rotary manipulandum 414, which may be a displayed as a cursor 1050 on the subject display 198. The motion of the rotary manipulandum 414 may be from zero to three-hundred sixty angular degrees, which may be translated with as representative motion, also from zero to three-hundred sixty angular degrees, in the form of a cursor 1050 on the subject display 198.

FIG. 10 presents further exemplary arrangements, wherein a linear manipulandum 416 is used. A linear manipulandum 416 may move in a linear motion 442. A linear manipulandum 416 may consist of a box-mounted slot 445 from which a handle 447 protrudes. The handle 447 is attached to circuit in the box 443 that transduces the movement of the handle 447 across the extent of the slot 445. The handle 447 may be grasped by the subject 192 and moved along the axis of the slot 445, which may move the cursor 1050 on the subject display 198. The movement of the cursor 1050 may be represented as a displayed linear cursor on the subject display 198. The displayed linear form of the cursor 1050 may move in a variety of means, including, but not limited to, a side-to-side motion or an up- and down motion, across a corresponding axis of the stimulus area 199.

FIG. 11 shows a further manipulandum arrangement, shown as a xy Cartesian manipulandum 418 that moves in the Cartesian coordinate system, which may be x-axis motion in the Cartesian coordinate system 444 or y-axis motion in the Cartesian coordinate system 446. The xy Cartesian manipulandum 418 may consist of a box mounted handle 449 that is attached to a xy Cartesian coordinate transducer circuit that registers the position of the handle's angular deflection. The box mounted handle 449 is tilted by the subject 192 to displace a cursor 1050 across the xy surface of the subject display 198; the xy surface of the subject display 198 may be shown from the upper left to the lower right of the subject display 198.

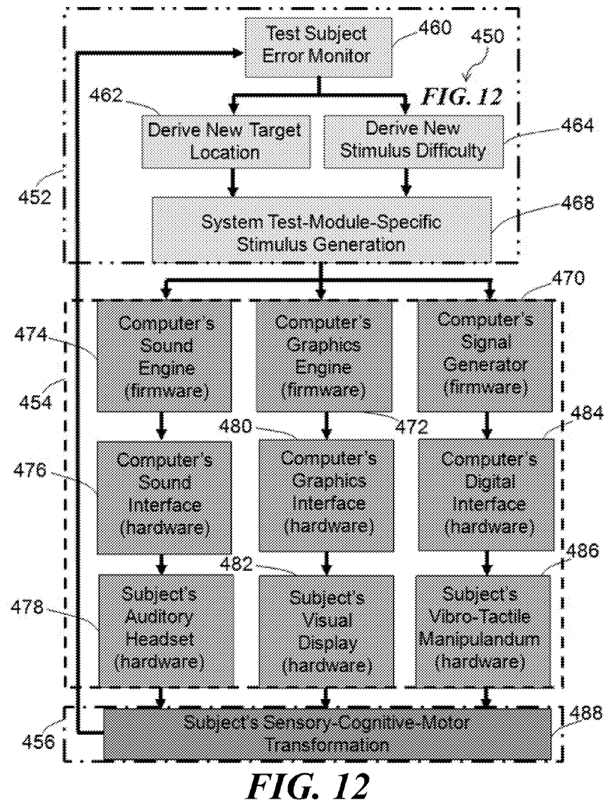

FIG. 12 portrays a block diagram of a stimulus generator 450, which may further comprise the system software 452, the application hardware configuration 454, and the system conceptualization of neural processing 456. Further, the block diagram of a stimulus generator 450 may combine hardware and software to produce a scene parameter.

The system software 452 may consider the test subject error monitor 460 towards both the steps of derive new target location 462 and derive new stimulus difficulty 464. The results of the steps of derive new target location 462 and derive new stimulus difficulty 464 may influence the step of system test-module-specific stimulus generation 468.

Further, the steps involved in the system software 452 may influence the steps involved in the application hardware configuration 454. More particularly, the results of the step of system test-module-specific stimulus generation 468 may be applicable towards each of the steps that are associated with the computer's sound's engine (firmware) 474, the computer's graphics engine (firmware) 472, and the computer's signal generator (firmware) 470.

The results of the step associated with the computer's sound's engine (firmware) 474 may be applicable towards the step associated with computer's sound interface (hardware) 476. The results of the step associated with the computer's graphics engine (firmware) 472 may be applicable towards the step associated with computer's graphics interface (hardware) 480. The results of the step associated with the computer's signal generator (firmware) 470 may be applicable towards the step associated with the computer's digital interface (hardware) 484.

Further, the results of the step associated with the computer's sound interface (hardware) 476 may be applicable towards the step associated with the subject's auditory headset (hardware) 478. The results of the step associated with the computer's graphics interface (hardware) 480 may be applicable towards the step associated with the subject's visual display (hardware) 482. The results of the step associated with the computer's digital interface (hardware) 484 may be applicable towards the step associated with the subject's vibro-tactile manipulandum (hardware) 486.

Further, the steps involved in the application hardware configuration 454 may influence the steps involved in the step of system test-module-specific stimulus generation 468. More particularly, the steps associated with either of the subject's auditory headset (hardware) 478, the subject's visual display (hardware) 482, or the subject's vibro-tactile manipulandum (hardware) 486 may be associated with the step of system test-module-specific stimulus generation 468.

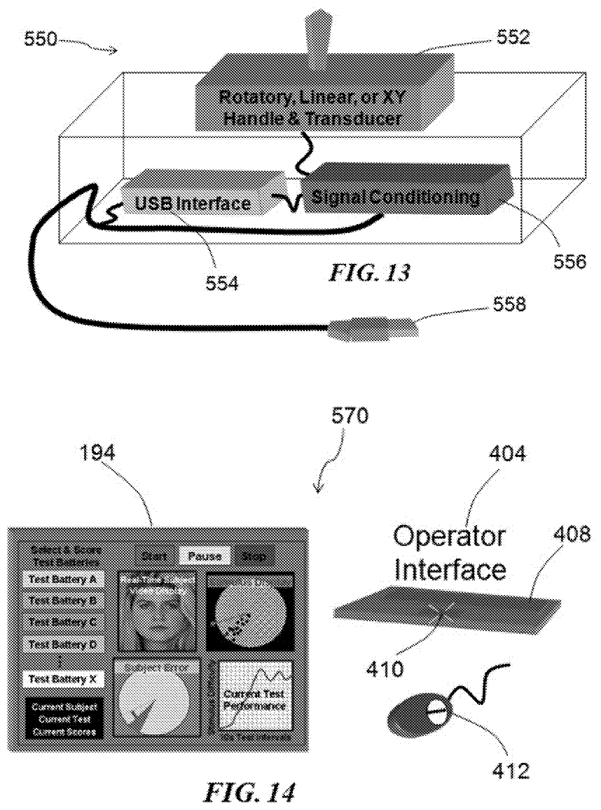

FIG. 13 shows a block diagram of the subject manipulandums 550, which represents the necessary components associated with the subject manipulandums 402. The components a of the block diagram of the subject manipulandums 550 may include, but is not limited to, the manipulandum handle and transducer 552, a USB interface 554, signal conditioning 556, and the USB connector to system computer 558. Further, the manipulandum handle and transducer 552 may be associated with either of the rotary manipulandum 414, linear manipulandum 416, or xy Cartesian manipulandum 418.

The output associated with the manipulandum handle and transducer 552 is coupled to the signal conditioning 556, which may either be applicable towards the USB interface or directly with the USB connector to system computer 558. The output associated with the USB interface is directly coupled to the USB connector to system computer 558.

FIG. 14 portrays an exemplary operator output interface 570, which may include, but is not limited to, an operator display 194 and an operator interface 404. The operator display 194 is shown in greater detail in FIG. 7 and its accompanying description. The operator interface 404 is shown in greater detail in FIG. 8 and its accompanying description. Further, the operator display 194 may include an exemplary real-time subject video display 332 for presenting tests of a series of scenes for use with the presently disclosed subject matter.

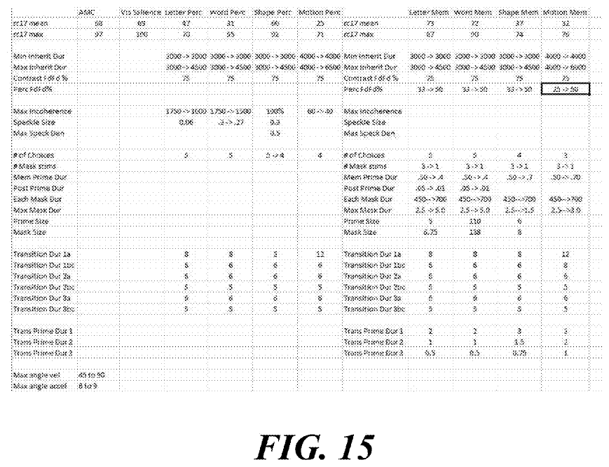

FIG. 15 depicts a sub-component of the operator display 194, the power user preset controls for visual movement module 600, which may serve as a graphical user interface with parameter adjustment sliders and buttons. The operator 190 may control the power user preset controls for visual movement module 600 in order to make changes to one, several, or all of the settings associated with the movement test 302. The power user preset controls for visual movement module 600 may include, but is not limited to, slider bars, with accompanying value ranges for the stimulus area 602, the stimulus speed 604, the range of dot speeds 606, the dot color 608, the background color 610, the mean dot luminance 612, the dot size (min, max) 614, the dot half-life (msec) 616, and the dot overlap (max %) 618.

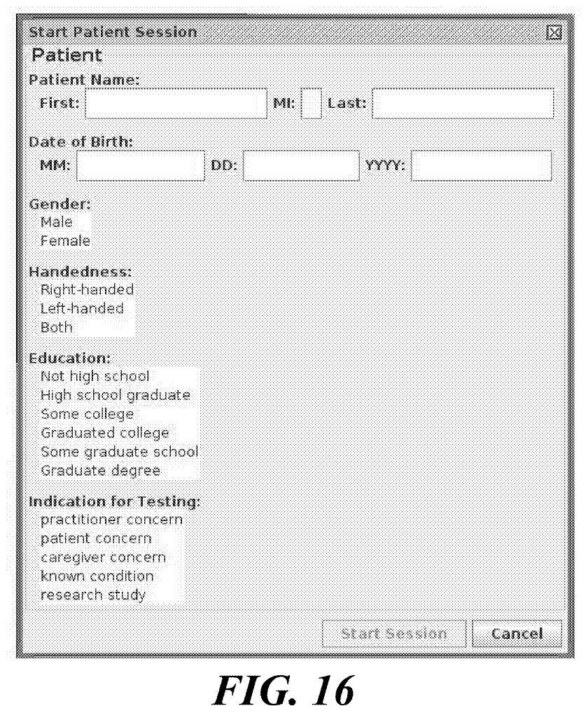

FIG. 16 presents a window in the operator display 194, which in addition to the option of select and score test batteries 340, may also include an exemplary subject demographics entry display 650. The operator 190 may enter subject demographics 652 for the subject 192 in the subject demographics entry display 650, which may be a sub-component of the operator display 194. The subject demographics may include, but are not limited to, the full name 660, the stated age 662, the date of birth 664, the gender identity 666, the racial identity 668, and the ethnic identity 670.

FIG. 17 shows a window in the operator display 194, which in addition to the option of select and score test batteries 340, may also include an exemplary subject medical history entry display 700. The operator 190 may enter the medical history 710 and the functional capacities 712 for the subject 192 in the subject medical history entry display 700, which may be a sub-component of the operator display 194. Further the medical history 710 may include, but is not limited to, medicinal allergies 720, other allergies (seasonal/food) 722, current medications 724, current supplements 726, current diagnoses 728, surgical procedures 730, planned surgeries 732, and history of trauma 734. Further the functional capacities 712 may include, but is not limited to, physical limitations 736, hearing impairments 738, visual impairments 740, movement difficulties 742, highest educational level 744, and preferred hand 746. The medical history 710 and the functional capacities 712 may contribute towards the quantitative assessment of functional impairment, and thereby may contribute towards the treatment for the subject 192.

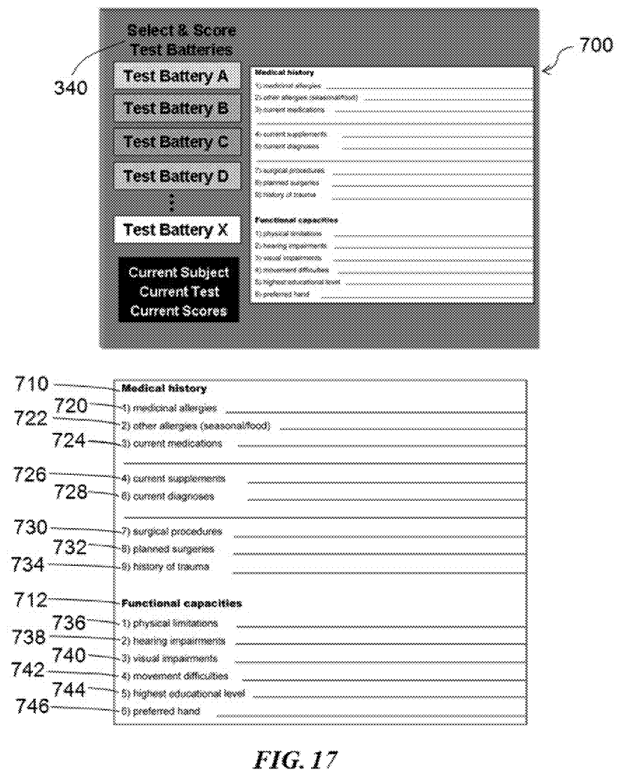

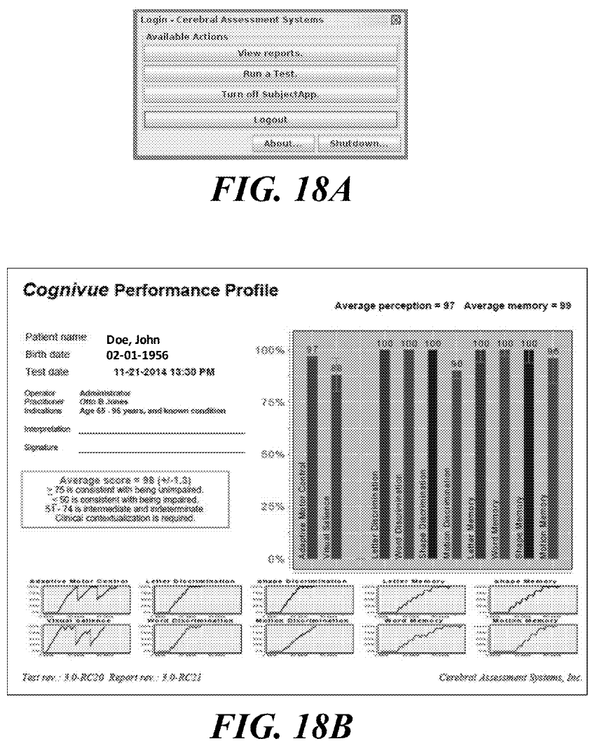

FIG. 18 shows a standard operations test scoring display 750, which may be a window in the graphical user interface for the display of the subject's current basic scores. The standard operations test scoring display 750 may be a display in addition to the option of select and score test batteries 340, which may be a part of the operator display 194.

The standard operations test scoring display 750 may further display a more detailed test scoring display 752, which may include, but is not limited to, the test subject output 760, the test module output 762, the saliency scores output 764, the mean over previous output 766, the interval scores output 768, and the percentage time at five seconds level output 770. Further, the test scoring display 752 may show current data associated with a current, particular test that may be for quantitative assessment of functional impairment.

Further, the mean over previous output 766 may be associated with the saliency scores output 764. Further, the percentage time at five seconds level output 770 may be associated with the interval scores output 768.

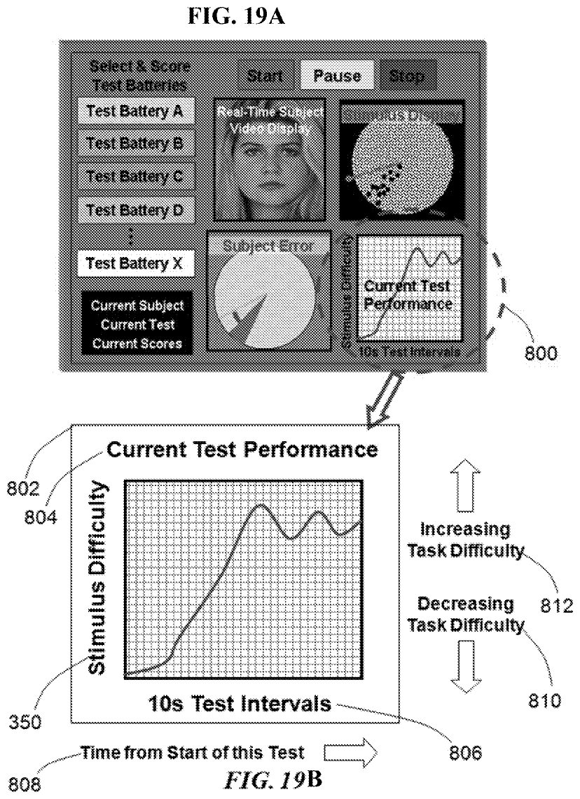

FIG. 19 shows a window in the operator display 194, which in addition to the option of select and score test batteries 340, may also include an exemplary standard operations dynamic performance display 800. The current test performance 802, which may be represented graphically as the graph of current of current test performance 804, which may be a graph of stimulus difficulty 350 versus ten seconds intervals 806.

Further, the ten seconds intervals 806 is an exemplary representation of the time from the start of this test 808. However, different time intervals may be represented on as the time from the start of this test 808 on the graph of current of current test performance 804.

Further, the graph of current of current test performance 804 may represent increasing task difficulty 812 with a higher value of stimulus difficulty 350. Further, the graph of current of current test performance 804 may represent decreasing task difficulty 810 with a lower value of stimulus difficulty 350.

Further, the current test performance 802 may be a more detailed representation of the standard operations dynamic performance display 800. Further, the current test performance 802 may be associated with the subject's response saliency function.

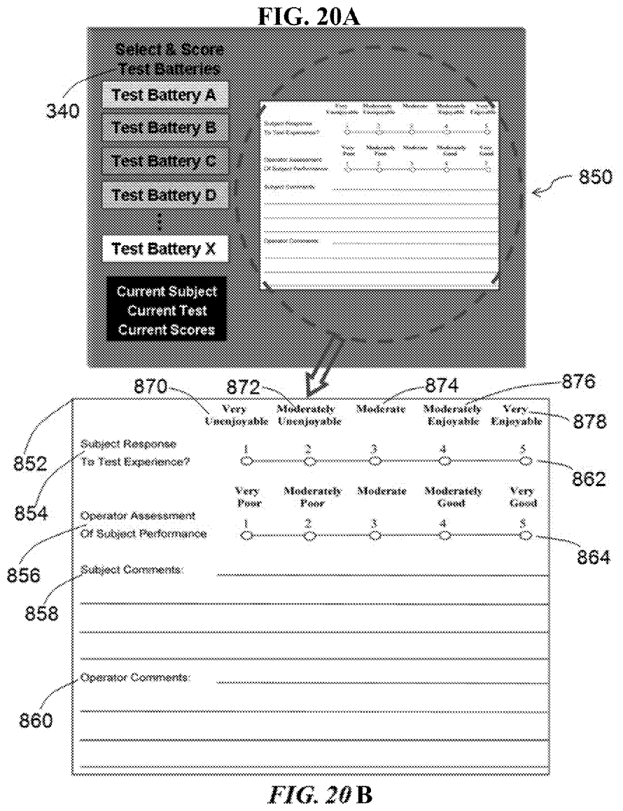

FIG. 20 shows a window in the operator display 194, which in addition to the option of select and score test batteries 340, may also include an exemplary operator comments entry display 850. The operator 190 may enter comments on the operator comments entry 852, which may be a sub-component of the operator comments entry display 850. The operator comments entry 852 may include, but is not limited to, prompts for subject response to test experience 854, operator assessment of subject performance 856, subject comments 858, and operator comments 860.

Further, the subject response to test experience 854 may be scored on a scale of subject response to test performance 862, which may be scored, but is not limited to being scored, from very unenjoyable 870 to moderately unenjoyable 872 to moderate 874 to moderately enjoyable 876 to very enjoyable 878. The operator assessment of subject performance 856 may be scored on a scale of operator assessment of subject performance 864, which may be scored, but is not limited to being scored, from very unenjoyable 870 to moderately unenjoyable 872 to moderate 874 to moderately enjoyable 876 to very enjoyable 878.

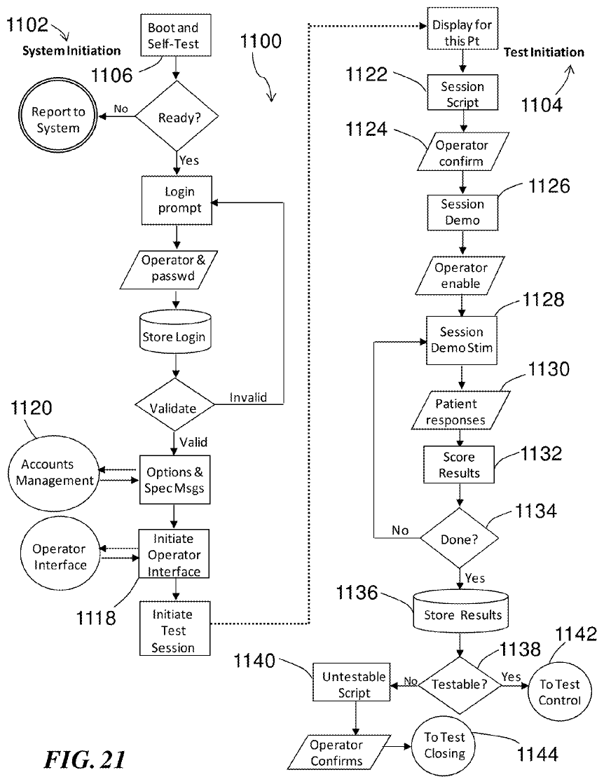

With reference to FIG. 21 through FIG. 78, the present disclosure includes multiple levels of system configurability implemented with an extensive multidimensional parametric control system with a large number of parametric adjustment controls. These parameters allow for the flexible specialization of the present disclosure across many application domains as well as the flexible specialization of the present disclosure to specific medical diagnoses and corresponding issues related to the wide variety of directly foreseeable applications of this technology.

The present disclosure allows for specialization of parameters with regards to tests included for specific applications, which may include, but is not limited to the following:
i. selection of specific tests for specific applications, such as a test battery that focuses on posterior cortical and sub-cortical function in applications regarding Alzheimer's Disease, and in contrast, a different test battery in screening of frontal lobe and temporal lobe function in applications regarding the fronto-temporal dementias;
ii. assessment of the underlying mechanisms for drug and toxin exposures, including applications for drug and toxin exposures that may be selected by experience acquired from implementation of the present disclosure;
iii. intrinsic configurability allows for implementing a broad-based, non-specialized screening, including measurement of the diverse dimensions of cognitive function across their respective ranges in the normal population, which may reflect the presence of, or the potential for, the wide range or neuropsychiatric disorders, or vulnerabilities to such disorders, seen in the healthy and functional population;
iv. a power-user test array configuration mode in which a specific sub-set of tests from the present disclosure may be included or excluded as best suited to the specific interests of the customer or for specific applications;
v. variable total duration of testing resulting from the intrinsic testing configurations.

Further, the present disclosure may provide for a complete, streamline workflow of experimental design, display calibration, data collection, and data analysis for the quantitative assessment of functional impairment.

Specialization of parameters for test configuration to be used in specific applications may include, but is not limited to, the following:
i. selection of all physical parameters of all the tests described in the present disclosure, including altering the speed of target motion, the rate of target saliency increase or decrease, spatial and temporal frequency composition of the stimuli and the nature of multi-modal stimuli, such as visual stimuli alone, auditory stimuli alone, hand-finger vibratory tactile stimuli alone, or any combination of those modalities as cues or distractors;
ii. parametric adjustment setting may include all aspects of the visual display, including, but not limited to, luminance, contrast, spatial (size of stimulus elements) and temporal (period of stimulus display) frequency composition, target position or change n position (movement);
iii. adjustment of aspects of the test subject's motor control medium, including but not limited to, adjusting response sensitivity, filtering subject response
iv. adjustment of aspects of auditory input to the subject, including, but not limited to, visual and/or auditory presentation of instructions, visual and/or auditory presentation of test stimuli, such as words or tones, the presentation of auditory stimuli as distractors, and the amplitude and filtering of auditory stimuli.
v. parametric adjustment due to qualitative assessment. Such parametric adjustment, including the ability to select parameters that are derived from demographic specification of the individual, which may include, but is not limited to, age, gender, medical history, drug treatments, or from the results of specific tests in a testing array sequence, which may include, but is not limited to, using a contrast sensitivity profile to alter the contrast at which all other visual stimuli will be presented, or using the speed and other subject movement parameters to alter the target movement parameters for all other tests. These subject performance dependent meta-parameters may be used as directly derived from that subject's or subject group's performance or may be algorithmically programmed.
vi. a power-user test parametric configuration mode in which computerized parameter adjustment sliders and buttons may be presented to allow for the adjustment of parameters as best suited to the specific interests of the customer or for specific applications.

Further, specialization of the testing configuration for applications to testing specific subjects may allow for the selection of a language in which instructions and linguistic stimulus cues that may be presented for testing subjects in their primary language or in a previously acquired secondary language.

Further, specialization of the testing configuration for applications to testing specific subject may allow for the selection of relevant cues such as geometric shapes or tones or such as objects and recognizable sounds rather than language cues in applications for age-appropriate, developmental, or acquired impairments of language processing.

Further, specialization of testing configuration for applications to testing specific subjects may allow for using an individual subject's scores from a previous testing session, at that site or another test site. Further, specialization of testing configuration for applications to testing specific subject may allow for using an individual subject's scores to select the test to be administered, which may potentially focus on abnormal or unreliable performance or on application specific selected performance capacities, for example detecting particular abilities affected by neurological disorders or testing particular abilities especially relevant to circumstances or tasks of special relevance to that subject. Likewise, test configuration parameters may be inherited from previous testing sessions to match those tests or to extend testing into a different parametric domain.

Further, specialization of the testing configuration for applications to testing specific subject may allow for operator entered alerts on areas of concern, which may be in response to patient complaints alerting the physician or operator regarding some function, such as memory, attention, or the controlled of skilled movements.

The present disclosure may include the extensive processing of subject performance data integrated with information from sources that may include: i) subject demographics, such as from scores standardized to normal for age or education, ii) subject characteristics from established or putative diagnoses or know treatment that may alter or focus analysis, such as with motor response in Parkinsonism, and/or iii) previous test scores, such as to focus on measuring improvement, stability or decline.

The present disclosure may include on-line data analysis, which may include the presentation and archiving of summary scores at the termination of the administration of each test. The scores from these tests may include: the mean saliency, as percent of maximum score, in last fifteen, ten, and five seconds of a test, the saliency at which the greatest percentage of time was spent in a test, the saliency at which the subject first lost track of the target. In another embodiment, the present disclosure may generate real-time score during the administration of each test.

The present disclosure may include off-line data analysis, which may include the derivation of a variety of dependent measures, including, but not limited to: i) the subject's response curve fit parameters to an asymptotic function, the salience level of that asymptote, and the time it takes to achieve that asymptote, ii) the area under the curve of the subject's response function, terminated by either a preset time, such as one-hundred seconds of testing or thirty seconds after the asymptote is reached, or the time to three peak/troughs in the response function or the time until a pre-selected cut-off is achieved, such as a saliency greater than ninety-five percentage, iii) comparative evaluations such as the differences between the measures of a subject's performance on a selected test versus that from another selected test, iv) comparative measures such as the differences between the basic measures of a subject on a test and the measures from a selected group of comparison subjects, such as the percentile scaled performance scores standardized for age, gender, or education.

More particularly, system initiation and test initiation, as applied to the quantitative assessment of functional impairment as described in the present disclosure, may be shown by way of illustration. FIG. 21 shows an embodiment of a testing flow process 1100 for the conceptual framework for quantitative assessment. At the start step of testing flow process 1100, the system initiation sequence 1102 may begin with the boot and self-test step 1106 and may proceed to initiate operator interface at step 1108. Upon receiving data entry input from the operator 190 via the operator interface 1120 during the initiate operator interface step 1108, the system initiation sequence 1102 may be completed.

The ensuing test initiation sequence 1104 may commence subsequently with the session script step 1122. Upon receiving operator confirmation 1124 the session demonstration 1126 begins with the session demonstration stimulus 1128. At step 1130 of patient responses, score results 1132 are recorded. Thereafter, done query 1134 may ascertain whether the session demonstration stimulus 128 has finished. If done query 1134 is no, then the test initiation sequence 1104 reverts back to the session demonstration stimulus 1128. If done query 1134 is yes, then the test initiation sequence 1104 proceeds with store results step 1136.

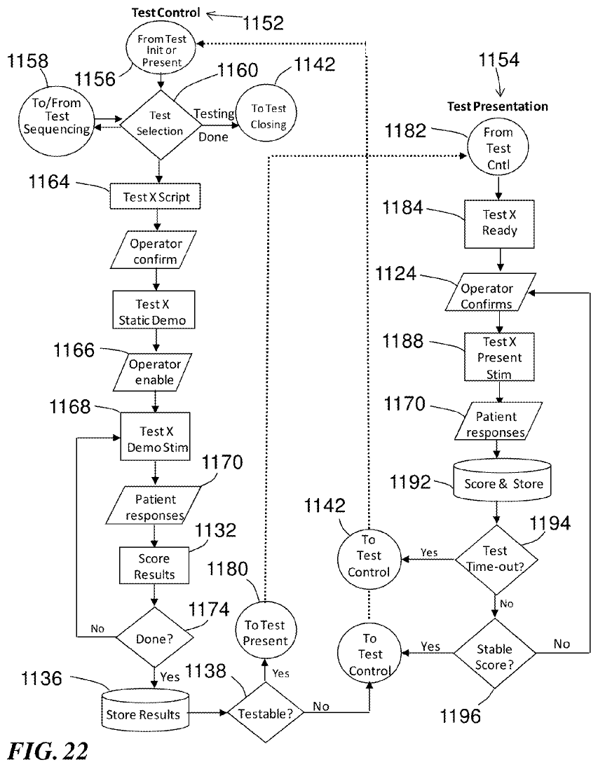

Thereafter, testable query 1138 may discern whether the store results are testable. If testable query 1138 is no, then the test initiation sequence 1104 determines a resulting untestable script 1140, and thereby proceeds to the test closing step 1144. If testable query 1138 is yes, then the test initiation sequence 1104 proceeds to the test controller that may operate as an integral or separate, local or remote, automatic or human decision maker interfaced to the testing device 1142, which is further depicted in FIG. 22 with more detailed steps.

More particularly, test control and test presentation, as applied to the quantitative assessment of functional impairment as described in the present disclosure, may be shown by way of illustration. FIG. 22 displays a sequence of test control steps 1152 and a sequence of test presentation steps 1154. At the test control step 1142 indicated in FIG. 21, the test initiation sequence 1104 may progress into the sequence of test control steps 1152. Initially after the test initiation or presentation step 1156, the sequence of test control steps 1152 proceeds to the query test selection 1160. Query test selection 1160 may search to allocate an appropriate test to/from test sequencing 1158. Upon achieving test selection 1160, the sequence of test control steps 1152 may proceed to test closing step 1142 under the assumption of no remaining tests. Further, upon achieving test selection 1160, the sequence of test control steps 1152 may proceed to the test script step 1164 under the assumption of remaining tests.

The operator enable step 1166 may promote the introduction of the test demonstration stimulus 1168. The sequence of test control steps 1152 may proceed with receiving input via patient responses 1170, for which the testing flow process 1100 records the score results 1132. If the sequence of test control steps 1152 does not complete score results 1132, then the sequence of test control steps 1152 continues with test demonstration stimulus 1168 in a control loop until the sequence of test control steps 1152 completes score results 1132.

Upon achieving score results 1132, the sequence of test control steps 1152 may proceed to the store results step 1136 and then to the testable query 1138. If testable query 1138 is yes, then the sequence of test control steps 1152 may proceed to step of to test presentation 1180 and initiates the sequence of test presentation steps 1154, starting with the step of from test control 1182. Then, at from test control step 1182, the sequence of test presentation steps 1154 may proceed with having a particular test x ready step 1184, followed by the step of operator confirmation 1124.

However, if testable query 1138 is no, then the sequence of test control steps 1152 may proceed to the step of to test control 1142. Afterward, the sequence of test control steps 1152 may revert back to the test initiation or presentation step 1156.

Upon receiving operator confirmation 1124, the sequence of test presentation steps 1154 may present a particular test x presenting a specifically selected stimulus 1188, and thereby promoting patient responses 1170. Subsequently, the patient responses 1170 may be recorded in the score and store step 1192, thereby prompting the test time-out query 1194. If test time-out query 1194 is no, then the sequence of test presentation steps 1154 proceeds to the query of stable score 1196.

However, if test time-out query 1194 is yes, then the sequence of test presentation steps 1154 may proceed to the step of to test control 1142, thereby reverting to the test initiation or presentation step 1156. If test time-out query 1194 is no, then the sequence of test presentation steps 1154 may present the stable score query 1196. If stable score query 1196 is no, then the sequence of test presentation steps 1154 may revert back to the step of operator confirmation 1124. However, if stable score query 1196 is yes, then the sequence of test presentation steps 1154 to the step of to test control 1142, may revert back to the test initiation or presentation step 1156.

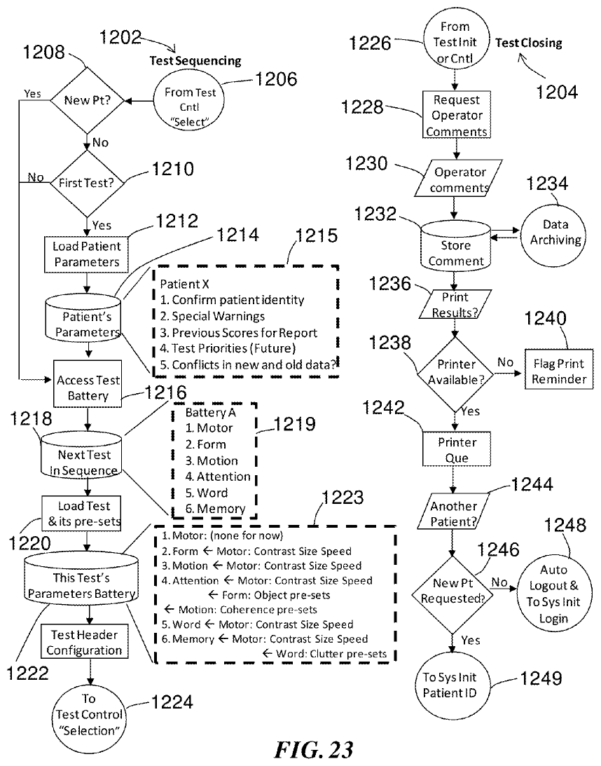

More particularly, test sequencing and test closing, as applied to the quantitative assessment of functional impairment as described in the present disclosure, may be shown by way of illustration. FIG. 23 illustrates the process flow of test sequencing 1202 in greater detail than as discerned at the step of from test control 1182 of FIG. 22. The subset of steps of from test control 1182 may begin with the from test control ‘select’ step 1206 of test sequencing 1202. Thereafter, a new patient query 1208 inquires whether a new patient has elected to participate in the test sequencing 1202. If no to new patient query 1208, then a first test query 1210 may be administered. If yes to new patient query prompt 1208, then the test sequencing 1202 proceeds to the step of access test battery 1216. Upon initiating first test query 1210, the test sequencing 1202 commences the step of load patient parameters 1212. Thereafter, the step of reviewing patient's parameters 1214 commences.

Further, the patient parameters reviewed 1215, which may be considered in the step of reviewing patient's parameters 1214, may include, but is not limited to the following: confirm patient identity, special considerations (e.g., age, gender, diagnosis), previous scores from earlier test reports, testing priorities based-on putative diagnoses or therapeutic interventions, and the need to resolve any conflicting or highly variable results of previous tests.

Immediately following step of reviewing patient's parameters 1214, the step of access test battery 1216 may commence. Thereafter, the progression of tests may be initiated in the step of next test in sequence 1218, which may include a particular test type 1219. Further, the particular test type 1219 may further include, but is not limited to, tests associated with any, some, or all of motor, form, motion, attention, word, and memory characteristics.

Further, the step of next test in sequence 1218 may start a sequence of the step of load test and its pre-sets 1220, which is immediately followed by an analysis step of this test's parameters battery 1222. More particularly, the step of this test's parameters battery 1222 may include, but is not limited to the details of type of parameter battery 1223, which is listed in list form detail in FIG. 23.

The final step of test sequencing 1202 may be the step of to test control ‘selection’ 1224, which returns the testing flow process 1100 back to the sequence of test control steps 1152, starting with the test initiation or presentation step 1156. Upon completion of tests and saving test data at the store results step 1136, the sequence of steps in test closing 1204 begins with the step of from test initiation or control 1226.

Thereafter, the step of request operator comments 1228 seeks operator comments 1230, which may be stored as store comments 1232 via a data archiving mechanism 1234. Subsequently, the user is prompted by the query of print results 1236 and the query of printer available 1238. If no to the query of printer available 1238, then the step of flag print reminder 1240. If yes to the query of printer available 1238, then the step of printer que 1242, immediately followed by the prompt of another patient 1244 to print another patient's test results.

Thereafter, a query of new patient requested 1246 may be initiated. If no to query of new patient requested 1246, then the step of auto logout and to system initiation login 1248 appears to the user. If yes to query of new patient requested 1246, then the step of to system initiation patient ID 1249 appears to the user.

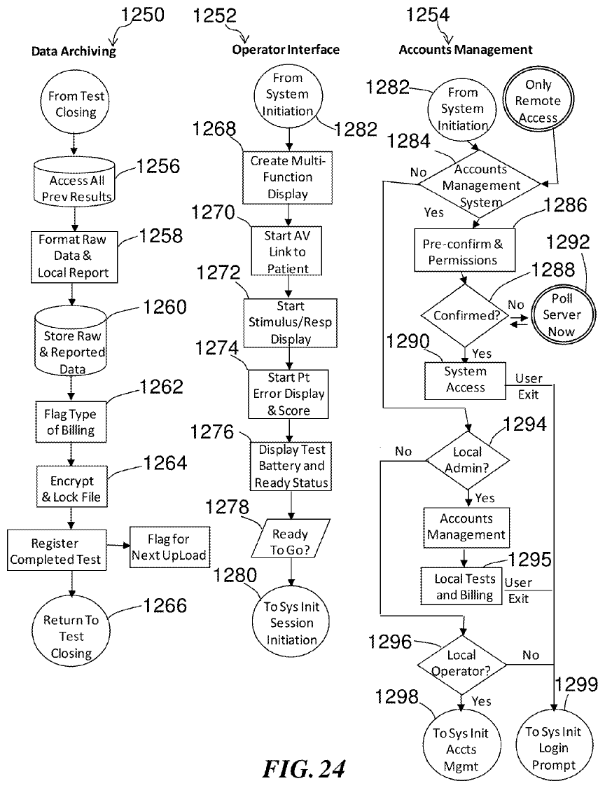

More particularly, data archiving, operator interface, and accounts management, as applied to the quantitative assessment of functional impairment as described in the present disclosure, may be shown by way of illustration. FIG. 24 shows sub-sequences of the testing flow process 1100, which may include the sequences of steps for data archiving 1250, operator interface 1252, and accounts management 1254. The process flow of data archiving 1250 may commence from the end of the sequence of steps in test closing 1204 as shown in FIG. 23.

Thereafter the steps for data archiving 1250 may commence with the step of access all previous results 1256, which are formatted in the step of format raw data and reported data 1258. Upon formatting the data from the test sequencing 1202, the data may be stored in the step of store raw data and reported data 1260. Thereafter, the process flow of data archiving 1250 may proceed with the step of flag type of billing 1262 and the subsequent step of encrypt and lock file 1264. The process flow of data archiving 1150 may end with return to test closing 1266. Some embodiments include data archiving includes secure archiving on the machine, data archiving on a remote location, data archiving of regulatory compliant de-identified data for use in other applications, and combinations thereof.

FIG. 24 also shows sub-sequences of the testing flow process 1100 for the operator interface 1252, which may begin with the step of from system initiation sequence 1282. Thereafter, the operator interface 1252 may proceed with the step of create multi-function display 1268, which is immediately followed by the step of start AV link to patient 1270. Next the operator interface 1252 may proceed the step of start stimulus/response display and score 1272, which initiates the subsequent step of start patient error display and store 1274 and the ensuing step of display the test battery and ready status 1276. Thereafter, the user may be prompted the step of ready to go 1278, which may be immediately followed by the step of to system initiation session initiation 1280.

Moreover, FIG. 24 also shows sub-sequences of the testing flow process 1100 for accounts management 1254, which may begin with the step of from system initiation 1282. Thereafter, the user may be queried with the step of accounts management system 1284. If no to the query of accounts management system 1284, then the follow-up step may be the query local admin 1294 to determine whether the user a local administrator. If yes to the query of asking whether the user is a local admin 1294, then accounts management 1254 may proceed to the step of local tests and billing 1295. However, if no to the query of asking whether the user is a local admin 1294, then accounts management 1254 may proceed to the step of the asking whether the user is a local operator 1190 via the query of local operator 1296. If yes to the query of local operator 1296, then accounts management may proceed to the step of to system initiation accounts management 1298; otherwise, accounts management may proceed to the step of system initiation and the presentation of a login prompt 1299.

Instead, if yes to the query of accounts management system 1284, then the testing flow process 1100 for accounts management 1254 may proceed with the step of pre-confirm and permissions 1286, which may be immediately followed by the step of confirming via the query confirmed 1288. If no to the query confirmed 1288, then the testing flow process 100 for accounts management 1254 may proceed to the step of poll system server now 1292. Instead, if yes to the query step of inquiring confirmed 1288, then the testing flow process 1100 for accounts management 1254 may proceed to the step of system access 1290. Thereafter step of system access 1290, accounts management 1254 undergoes user exit mode and ends the accounts management 1254 at the system initiation login prompt 1199.

With reference to FIG. 25 through FIG. 78, the present disclosure includes a screening test battery with high stimulus-response compatibility (the stimulus has a self-evident relationship to the required response; e.g., a stimulus on the left or right of the screen is highly compatible with push-button responses through a device that has one button on the left and one button on the right) to facilitate engaging test subjects while surveying a range of functional domains to detect and quantify a variety of functional impairments.

The fundamental stimulus response contingency common to all of these tests is the segmental presentation (some part of the overall display) of a stimulus in the context of relevant distractors (other parts of the overall display) to evoke the subject's positioning of a cursor to indicate the local stimulus.

In one embodiment of the present disclosure, the tests are organized to captures all aspects of sensory input, cognitive/affective interpretation and transformation, and motoric response control, herein called sensory-motor neurocognitive assessment, which may also be known as sensory-cognitive-affective-motor assessment. The present disclosure may couple sensory stimulation with the recording of motor responses to assess cerebral cortical function. The stimulus-response patterns are recorded in the context of the different tests, which thereby allow for: 1) the quantification of fundamental sensory and motor functions, 2) the quantification of multiple levels of high cognitive function and of affective (emotional) function by measuring its influence on motor function, and 3) the detection of impairments or improvements in any of these functions.

The tests may provide a graph of stimulus saliency over time achieved by the test subject in tasks of sensory-motor neurocognitive-affective assessment task (e.g., success leads to more difficult tasks and stimuli so performance capacity is reflected in the difficulty reached in testing). Further, the tests of the present disclosure may characterize functional impairment in sensory-motor neurocognitive-affective assessment through evaluation of quantifiable characteristics.

One such quantifiable characteristic of impairment in sensory-motor neurocognitive-affective assessment may be high latency to the subject's optimal function in a sensory-motor neurocognitive-affective assessment task, which may be a less steep sensory-motor neurocognitive-affective assessment function. This latency measure may be obtained by sudden changes in the stimulus-response paradigms that require the test subject to rapidly adapt to those changes (e.g., sudden reversal of subject response wheel directional relationship to screen cursor direction (i.e., subject must turn wheel counterclockwise to turn the cursor clockwise).

Another such quantifiable characteristic of impairment may be high variability of optimal function during a sensory-cognitive-affective-motor neurocognitive assessment task, which may be larger terminal fluctuations (i.e., subject's speed, accuracy, or other response measures) becomes more variable as the stimulus becomes less readily discriminated (more difficult to recognize). Yet another such quantifiable characteristic of impairment may be low enhancement of neurocognitive assessment function, particularly being steeper or higher, by valid cueing. The term “valid cueing” may refer to providing a stimulus that allows the subject to have fore-knowledge of a subsequent stimulus, accessing perception, attention, and memory that may be able to provide a higher resolution view of sensory-cognitive-affective-motor function.

Another such quantifiable characteristic of impairment may be either an enhancement or a diminution of neurocognitive assessment, (e.g., by comparing responses to valid and invalid cueing. The term “invalid cueing” representing test conditions in which attention or memory provides incorrect information about the nature or content of the stimulus in a motor neurocognitive assessment.

Further, an embodiment of the present disclosure may include a pattern of visual motion associated with a stimulus area 199 that may be translational motion, rotational motion, radial motion, or motion that may be in a combination of translational, rotational, and radial motion. Further, the motion associated with the stimulus area 199 may be random in nature, as governed by a variety of visual noise generation algorithms.

Further, another embodiment of the present disclosure may include continuous feedback adjusted stimulation. Wherein, a spatial sub-section of the stimulus is distinct from the remainder of the stimulus by virtue of a gradient or boundary of difference in a single stimulus parameter or a selected set of stimulus parameters. Such a boundary may reflect a single step change at some edge, multiple step changes at successive distances steps away from the target's center, or a graded function with distance from the center of the target.

Further, the tests of the present disclosure may continually change the location of the target in the stimulus field. The present disclosure may include a continually changing response from the subject 192. The target location may change by either angular displacement around an axis of rotation, displacement along a single axis or any fixed or varying orientation, or displacement along multiple axes, such as horizontal and vertical axes.

Additionally, the saliency of the target, which refers to perceptual distinctness of the target from the background and from other stimulus elements, may be continually change during a neurocognitive assessment task to alter the difficulty of the task and establish the neurocognitive assessment response function of the subject 192 in that assessment domain.

Further, in the tests of the present disclosure, the cursor 1050 may itself be the target zone of one of two concurrently presented stimuli, in separate display areas or superimposed on a single display area, in which the target position is independently manipulated by an algorithm (as in a single stimulus test) and the other target is independently manipulated by the test subject or patient as with the cursor in a single stimulus test 1050. A computer system 200 may control the saliency associated with the cursor stimulus 1050, thereby allowing the subject 192 to perform two well-defined neurocognitive tasks concurrently, a circumstance which may be associated with dual task interference or dual task enhancement. More particularly, the subject 192 may be asked to align one target area with another target area during functional impairment testing associated with dual task interference (e.g.: Two concentric annular target areas, or two parallel linear target areas, are presented simultaneously. Each target area contains a categorical target imbedded in a field of non-target “foil” stimuli. The two target areas may contain targets and foils of the same, or of different stimulus categories. Within each target area, a target and foils are presented as the salience of those elements is parametrically co-varied, or independently varied, in relation to how well the subject manipulates the response interface device to align the target in one area with the computer controlled moving location of the target in the other area).

Further, during the tests of the present disclosure, the subject performance controls the rate and direction of change in target location and saliency. The speed, maximum acceleration, and rate of direction changes may be increased when the subject 192 if off target and decreased when the subject 192 is on target. The saliency may be increased when the subject 192 if off target, decreased when on target; the rate of change is proportionate to the size and duration of subject error.

Additionally, the duration of testing may be controlled by the size and duration of subject error. More particularly, sustained, stable scores may lead to earlier termination of testing. Multiple oscillations of scores around a stable level may lead to the termination of that specific test. The inability to capture the target at any saliency may lead to the termination of that specific test.

Further, exemplary neurocognitive assessment response characterization protocols may be initiated using configurations informed by previous tests. Motor control response parameters, such as the maximum speed, maximum acceleration, and minimum direction reversal interval generated by a subject, may be used to establish, in a particular test or across tests, the then used standards for parameters used in subsequent tests. Further, sensory contrast sensitivity measures may be determined, in one or more sensory modality or sub-modality, and used in subsequent tests to provide each subject 192 with individually standardized stimuli in later tests. Further, neurocognitive assessment neural processing measures may be used for comparison to adjust scores in attentional and memory manipulations superimposed on those tests to further inform the assessment in those tasks, degradation protocols, and tasks.

Further, another embodiment of the present disclosure may be to operate a system for quantitative assessment of functional impairment with minimal intervention. The present disclosure may include artificial intelligence capabilities to enable dynamic testing (e.g., real-time test selection based on previously entered or obtained information). Further, each test of the present disclosure may include an ability to dynamically respond to actions of subject 192. Thus, each test in the present disclosure may shorten or lengthen itself automatically in response to the actions taken by the subject 192.

In one embodiment, ten tests may be administered to assess functional impairment of the subject 192. Further, in one embodiment, the tests may be administered in the order described below. However, the methods in accordance with the embodiments of the present disclosure may include the performance of any other subset of the ten tests which may be administered in any order. Further, the tests may encompass present and future known equivalents to the known components referred to herein by way of illustration.

FIG. 25 illustrates the initiation of the dynamic contrast test, which evaluates visuo-motor responses by analysis of the sensory-cognitive-affective-motor function in the domains of target movement speed, acceleration, and direction reversal. A patch of high contrast may be comprised of individual elements, which includes, but is not limited to, circles, checkerboard, or stripes. The individual elements, herein called dots, may be equally displaced to either high or low luminance levels and may be distinguished from an intermediate luminance background otherwise filling the stimulus area.

The starting phase of the dynamic contrast test 1300 may initiate movement of a high color/contrast patch on a stimulus area 199. An equal number of darker dots 1304 and lighter dots 1306 may be presented within the background stimulus area 1308, which may be surrounded by the high or low relative luminance border 1302. The darker dots 1304 and lighter dots 1306 may be randomly assigned in a wide or narrow range of sizes, thereby assessing spatial frequency dependence distributed randomly, as white noise, pink noise, or other spatial frequency distributions formed by dots displayed on the screen. A high color/contrast/spatial frequency patch, which may be an active stimulus target segment 1310, which may move within the stimulus area 199. The active target segment 1310, which may be a twenty-five degrees section, that may be manipulated to make the target segment larger or smaller, within the annular/circular/linear stimulus area 1302, may contain a number of relatively higher contrast level darker and/or lighter dots 1312 with the remainder of the stimulus area containing and relatively lower contrast level darker or lighter dots 1314.

The higher contrast target dots segment of the stimulus area 1304 may vary in contrast relative to the lower contrast non-target remainder of the stimulus area and the position of the higher lighter-contrast dots 1306 fade in and may vary in position within the stimulus area. In addition, the overall luminance of the stimulus area, and the non-stimulus area sections of the display screen, may vary separately 1308. The stimulus area's dots may be displayed with randomly assigned life time periods that are chosen within a range of time intervals creating a continually changing pattern of dots. A test developer, implementing modifications of test parameters 190 may pre-set the overall luminance brightness level of the neutral-contrast background stimulus area 1308, the number of higher-contrast dots 1304 and lower-contrast dots 1306 within the circular border 1302, and the relative color of the of the neutral or intermediate-contrast background stimulus area 1308 relative to the color of the higher and lower-contrast dots 1304 1306, and the maximum size of the higher-contrast target dot area 1304 and lower-contrast non-target dot area 1306.

A stimulus generator 450 supplies an algorithm that may be applied to relatively higher contrast level target dots 1312 and relatively lower contrast level non-target dots 1314 within the active stimulus target segment 1310, which may make the relatively higher contrast level dots 1312 achieve greater perceptual salience compared to the dots in the lower or neutral-contrast non-target stimulus area or non-stimulus area background 1308 1314 1308.

The developer 190 may pre-set settings for the active stimulus target segment 1310, the brightness level of the overall stimulus area segment 1310, the number of relatively higher contrast level target dots 1312 and relatively lower contrast level non-target dots 1314 within the active stimulus target segment 1310, the relative color of the of the active stimulus target segment 1310 relative to the color of relatively higher contrast level target dots 1312 and relatively lower contrast level non-target dots 1314, and the maximum diameter of the relatively higher contrast level target dots 1312 and relatively lower contrast level non-target dots 1314.

During the starting phase of the dynamic contrast test 1300, the active stimulus radial segment 1310 may generate the highest contrast level for either, or both, the relatively higher contrast level dots 1312 and the lowest contrast level for the relatively lower contrast level dots 1314 within the active stimulus radial segment 1310. Then, the active stimulus target segment 1310 may begin to move continuously, and while doing so, the active stimulus target segment 1310 may change direction in either a clockwise or counterclockwise direction and/or it can accelerate or decelerate.

The subject 192 may be asked to identify and to parallel the movement of the active stimulus radial segment 1310 using a subject manipulandum 1402 during the starting phase of the dynamic contrast test 1300. The subject's control and movement of an subject manipulandum 1402 may be tracked on the subject display 198 with a cursor 1050. The active stimulus radial segment 1310 may be tracked with the cursor 1050 via the subject's control.

As the active stimulus target segment 1310 moves around the neutral-contrast background stimulus area 1308, the contrast level within the active stimulus target segment 1310 may begin to change along with the location, direction, and speed of the active stimulus target segment 1310. As the contrast level of the active stimulus target segment 1310 begins to decline, the subject 192 will find it to be more difficult to follow the movements of the active stimulus target segment 1310. Therefore, the operator 190 may gauge an approximate threshold for the relative contrast level of the active stimulus target segment 1310 that the user can decipher.

FIG. 26 shows the intermediate phase of the dynamic contrast module test 1320, a phase marked by a discontinuous nature. During this discontinuous phase, the active stimulus target segment 1310 may move about in a discontinuous fashion, beginning with fade-out stage of a low contrast level for the active stimulus radial segment 1310 at a level equal to or lower than the initial contrast level of the starting phase of the dynamic contrast test 1300.

During this fade-out period, the active stimulus target segment 1310 may fade-out initially (becoming progressively less perceptually salient). Subsequently, the active stimulus target segment 1310 may fade-in (become progressively more perceptually salient) with the relatively higher contrast level target dots 1312 1314 within the active stimulus target segment 1310 being recreated in contrast conditions according to original randomization conditions; however, the recreated relatively higher contrast level target dots 1312 1314 are moved, via a motion herein analogous to a jumping motion, to a new location within the neutral-contrast background stimulus area 1308, which is filled with-contrast dots 1304 306 and may also be surrounded by the higher or lower contrast border 1302.

Whenever the subject 192 moves the subject manipulandum 402, the cursor 1050 may be moved by the subject to track the target active stimulus target segment 1310; if the subject 192 can successfully track the active stimulus target segment 1310 within predetermined limits, a separate signal, such as a bright flash and beep that may signal or may confirm the action of the subject 192. The intermediate phase of the dynamic contrast test 1320 may continue with further jumps until the test subject's stimulus-response performance 190 defines a further refined threshold; subsequent restarting of the intermediate phase of the dynamic contrast test 1320 may continue at varying levels of contrast and rates of contrast increase and decrease, resulting in a repeat process until that subject's perceptual threshold may be estimated.

FIG. 27 illustrates the termination phase of the dynamic contrast test 1322, during which the subject 192 may no longer be able to distinguish the presence of an active stimulus target segment 1310 within the lower or neutral background of the stimulus area 1308. At this point, the final movement dynamics of the subject's response manipulandum, and the related movement of cursor 1050, may mark the critical threshold as part of that subject's performance score, for which the data of the threshold in used in the ensuing tests. Immediately following the critical threshold point, the higher-contrast dots 1304 and lower-contrast dots 1306 may fill the entire stimulus area 1308, which may be surrounded by the border on the display 1302.

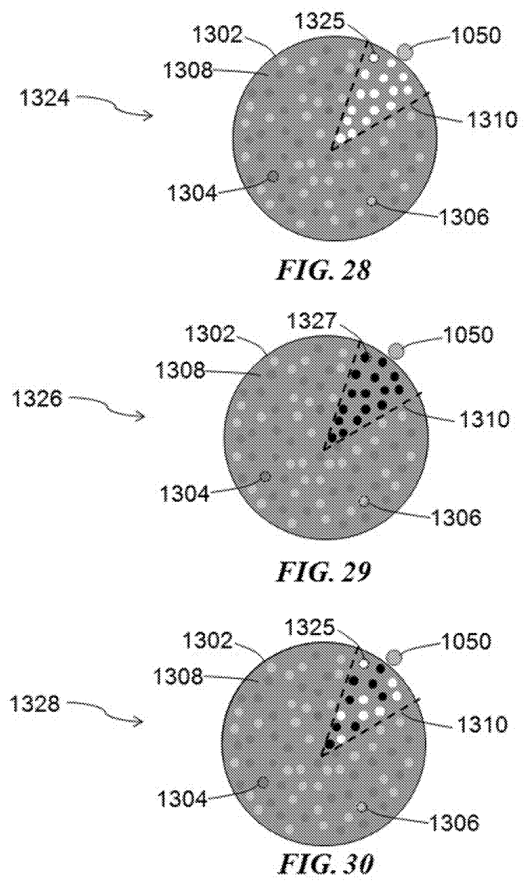

FIG. 28 depicts the starting phase of the visual contrast sensitivity test 1324, which may involve the implementation of a patch of high luminance elements 1325 onto an active stimulus target segment 1310, which may be within a high contrast border 1302. The patch of high luminance elements 1325 may include, but are not limited, to being circles, checkerboard, or stripes. The individual elements may be distinguished from intermediate luminance background elements to vary saliency. The subject 192 controls the position and movement of a cursor 1050 to match that of the target.

During the starting phase of the visual contrast sensitivity test 1324, high luminance elements 1325 may be distinguished from the darker-contrast dots 1304 and lighter-contrast dots 1306 that may be randomly assigned in the neutral-contrast background stimulus area 1308.

FIG. 29 depicts the intermediate phase of the visual contrast sensitivity test 1326. The high luminance elements 1325 may be automatically transitioned to becoming low luminance, thereby becoming low luminance elements 1327, during the intermediate phase of the visual contrast sensitivity test 1325. The transition to becoming low luminance elements 1327 may enable the subject 192 to determine the threshold.

FIG. 30 illustrates the termination phase of the visual contrast sensitivity test 1328, during which the subject 192 may be presented with both a mixed luminance elements, comprising both high luminance elements 1325 and low luminance elements 1327, within the active stimulus radial segment 1310. During the process of the stimulus radial segment 1310 gradually presenting a mixed luminance, the subject 192 may be cued to determine the threshold to achieve an equal number of high luminance elements 1325 and low luminance elements 1327 within the active stimulus radial segment 1310. At the point when the subject 192 may determine an equal number of high luminance elements 1325 and low luminance elements 1327, the final location of the cursor 1050 may mark the critical threshold, for which the data of the threshold in used in the ensuing tests.

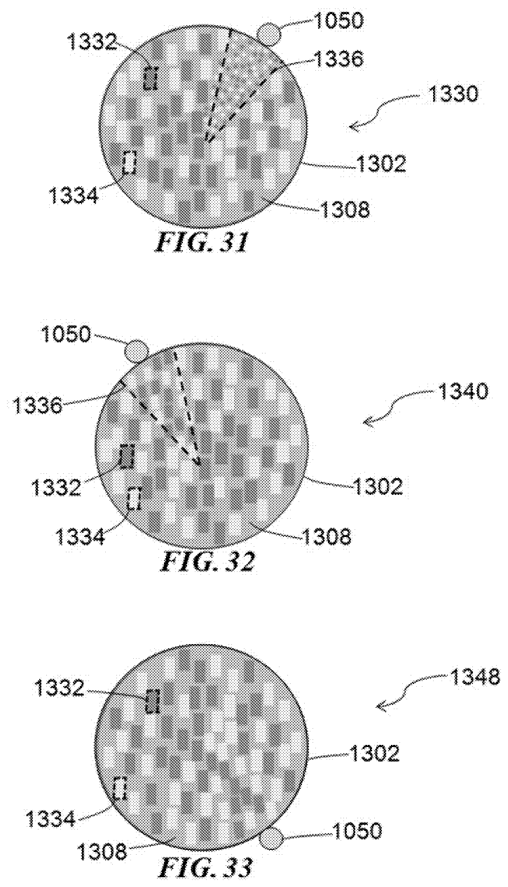

FIG. 31 depicts the initiation of the visual form discrimination test, during which patches of regular shapes may be distorted to distinguish target area shapes from their background. During the visual form discrimination test, patches of regular shapes may be distorted to distinguish the target area shapes from the background. The patches of regular shape may be distorted in a manner including, but not limited to, size, shape, aspect ratio, line thickness, and/or orientation. The subject 192 may control the position and movement of cursor 1050 to match that of the target.

During the starting phase of the visual form discrimination test 1330, an equal number of darker-contrast rectangles 1332 and lighter-contrast rectangles 1334 may be presented within a neutral-contrast background stimulus area 1308, which may be surrounded by a stimulus area border 1302. The darker-contrast rectangles 1332 and lighter-contrast rectangles 1334 may be randomly assigned in sizes of one unit length width and three unit lengths height across the screen. An active visual form module stimulus radial segment 1336, which may be a twenty-five degrees section, larger or smaller if being dynamically modulated, within the circular border 1302, contains a number of relatively higher contrast level darker rectangles 1332 and relatively lower contrast level lighter rectangles 1334.

A test developer 190 may pre-set the brightness level of the neutral-contrast background stimulus area 1308, the number of darker-contrast rectangles 1332 and lighter-contrast rectangles 1334 within the circular border 1302, the relative color of the of the neutral-contrast background stimulus area 1308 relative to the color of the darker-contrast rectangles 1332 and lighter-contrast rectangles 1334, and the maximum diameter of the darker-contrast dots 1304 and lighter-contrast dots 1306.

The darker-contrast rectangles 1332 and lighter-contrast rectangles 1334 may fade in and out in the neutral-contrast background stimulus area 1308 with assigned life time periods that may chosen within a timed interval set between thirty-six and one-hundred eight frames at seventy-two frames per second with emergence and fading occurring over three frames. Further, the darker-contrast rectangles 1332 and lighter-contrast rectangles 1334 may fade in and out in the neutral-contrast background stimulus area 1308 while moving to random new positions.

The subject 192 may be asked to identify the active visual form module stimulus target segment 1336 using a manipulandum 402, during the starting phase of the visual form discrimination test 1330. The subject's control and movement of a subject manipulandum 402 may be tracked on the subject display 198 with a cursor 1050. The active visual form module stimulus radial segment 1336 may be tracked with the cursor 1050 via the subject's control.

FIG. 32 displays the intermediate phase of the visual form discrimination test 1340, a phase marked by a discontinuous nature. During this discontinuous phase, the rectangular elements within the active visual form module stimulus radial segment 1336 may vary in size, shape, and orientation while the active visual form module stimulus radial segment 1336 moves continuously around the circular border 1302 with varying levels of distinctiveness. More particularly, the active visual form module stimulus radial segment 1336 may move continuously around the circular border 1302 while accelerating or decelerating and/or moving clockwise or counterclockwise; furthermore, the rectangular elements within the active visual form module stimulus radial segment 1336 may change direction of movement from clockwise to counterclockwise or vice-a-versa.

The subject 192 may be asked to match the movement of the active visual form module stimulus target segment 1336 using a cursor 1050, which a may be physical interface akin to a wheel or a joystick, during the intermediate phase of the visual form module test 1340. Subsequently, the active visual form module stimulus target segment 1336 fades-in with the relatively higher contrast level darker or lighter than background rectangles 1332 1334 within the active visual form module stimulus target segment 1336 being recreated in contrast conditions according to original randomization conditions; however, the re-created relatively higher contrast rectangles 1332 1334 may be moved, via a motion herein analogous to either a drifting or jumping motion, to a new location within the neutral-contrast background stimulus area 1308.

Whenever the subject 192 moves the cursor 1050 into the target active stimulus segment 1310, an instant bright flash and beep may signal and may confirm the action of the subject 192. The intermediate phase of the visual form module test 1340 may continue with further jumps until the operator 190 develops a further refined threshold; subsequent restarting of the intermediate phase of the intermediate phase of the visual form module test 1340 may continue at varying levels of contrast and rates of contrast increase, resulting in a repeat process until an ensuing threshold is attained.

FIG. 33 illustrates the termination phase of the dynamic contrast discrimination test 1348, during which the subject 192 may no longer distinguish the presence of the active visual form module stimulus radial segment 1336 within the neutral-contrast background stimulus area 1308. Hence, the darker high contrast 1332 and lighter high contrast rectangles 1334 may be distributed throughout the entire the neutral-contrast background stimulus area 1308, which may be surrounded by the border 1302. At this point, the final location and movement of the subject's response manipulandum and the related location and movement of the cursor 1050 may mark the critical threshold, for which the data of the threshold may be used in the ensuing tests.

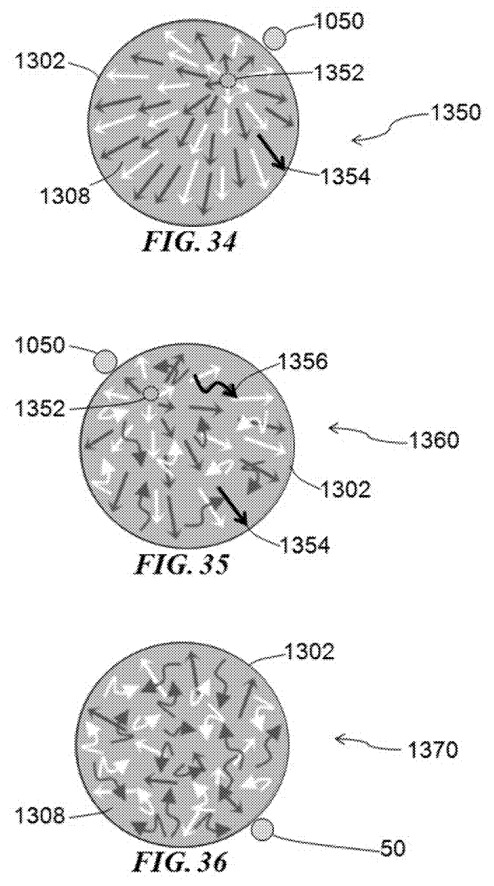

FIG. 34 depicts the initiation of the visual motion discrimination test, during which spots move in a planar, radial or circular pattern or create a motion defined edge or a point. The subject 192 may control the position and movement of a cursor 1050 to match of the target. During the visual motion discrimination test, the salience of the target may be decreased by shifting more elements to random motion with fewer elements moving in compliance with the pattern of movement.

The starting phase of the visual motion discrimination test 1350 may include segmental presentations of a radial center of motion in optic flow. An equal number of darker high contrast dots 1304 and lighter high-contrast dots 1306 may be presented within a neutral-contrast stimulus area 1308, which may be surrounded by a border 1302. The perceptual salience of the motion pattern may be manipulated by a variety of stimulus parameters. For example, The contrast levels for the darker high-contrast dots 1304 and lighter high-contrast dots 1306 may be set two confidence intervals above the threshold established in the starting phase of the dynamic contrast test 1300. The darker high-contrast dots 1304 and lighter high-contrast dots 1306 may move in an outward radial pattern 1354 by moving away from a focus of expansion 1352, which may be a designated point within the stimulus area 1302 or toward a focus of contraction which may be a designated point within the stimulus area. Alternatively, as detailed below, the perceptual salience of a motion pattern stimulus may be manipulated by replacing moving elements in the pattern with randomly placed elements or elements moving independently of the pattern.

More particularly, the focus of expansion, or the focus of contraction 1352 may be located anywhere within the stimulus area; however, the eccentricity of the focus of expansion or contraction 1352 may be pre-set. Further, the darker high-contrast dots 1304 and lighter high-contrast dots 1306 may be randomly assigned in size in the range of three degrees or smaller, thereby maintaining a pink noise spatial frequency composition of dots across the screen. Moreover, the control variables may include background brightness neutral-contrast background stimulus area 1308 and dot density, color, spatial frequency, and speed of the darker-contrast dots 1304 and lighter-contrast dots 1306. The ratio of dots that may be moving in the pattern to the number of total dots may be known as the coherence ratio. Of note, the ratio may be full coherence, with a ratio of one to one (all dots move in the pattern), or no coherence (all dots move randomly), with a ratio of zero to one.

The darker-contrast dots 1304 and lighter-contrast dots 1306 may fade and emerge with a random lifespan between thirty-six and seventy-two frames with three frames for emergence and three frames for fading. The speed of the darker-contrast dots 1304 and lighter-contrast dots 1306 may be a sine function of the angular distance from the focus of expansion 1352 the product of which may be algorithmically manipulated a assess subject sensitivity to direction and speed gradients within the pattern. The starting phase of the visual motion discrimination test 1350 may begin with full coherence where the subject 192 can all points moving in an outward radial pattern 1354 away from the singular point known as the focus of expansion 1352.

FIG. 35 shows the intermediate phase of the visual motion discrimination test 1360, a phase during which the focus of expansion 1352 may move with varying movements of coherence, location, direction, and speed. The darker-contrast dots 1304 and lighter-contrast dots 1306 may move in an outward radial pattern 1354 or in a random fashion 1356 from a frame to another frame. The subject's cursor may be identified as a twenty-five-degree cursor segment, that may be modulated from higher to lower sizes. The subject 192 may move the manipulandum so that the cursor moves 1050 to the focus of expansion or contraction 1352.

When the subject 192 moves the cursor 1050 to enter the twenty-give degree segment, then the intermediate phase of the visual motion discrimination test 1360 may produce a bright flash and beep of may transition directly to the next stimulus or task. Starting with a high level of coherence (high SNR), the focus of expansion 1352 may move in a discontinuous fashion, jumping motion around the stimulus area 1302 with potential changes in coherence with each fade and emergence sequence; with each such jump, the coherence level increases (gets easier) if the subject shows poor performance and decreases (gets harder) if the subject shows good performance.

FIG. 36 illustrates the termination phase of the visual motion discrimination test 1370, during which the subject 192 may no longer distinguish the location of the focus of expansion or contraction 1352. Hence, the darker-contrast dots 1304 and lighter-contrast dots 1306 may fill the entire the neutral-contrast background stimulus area 1308, which may be surrounded by the circular border 1302. At this point, the final location of the cursor 1050 may mark the critical threshold, for which the data of the threshold in used in the ensuing tests. Ultimately, this threshold may be achieved by successively constraining the starting coherence and the rate of increase.

With reference to FIGS. 34, 35, and 36, may include, but is not limited to, presentations of a radial center of motion in optic flow, which may include the focus of expansion 1352 or contraction in the stimulus area 199. comparable stimulus sets may be composed of planar or circular patterns of movement, wherein the subject 192 may orient a cursor 1050, which may include, but is not limited to, any of the previously described and illustrated subject interface response devices. Depending on the stimulus set and the behavioral task, the subject is to move the cursor in the direction of motion or to a motion define point or edge. Further, equivalents of the present subject matter may present a circular pattern of motion with the center of rotation moving around the stimulus area 199 just as the focus of expansion 1352 may move around in a radial optic flow field. Further, the circular and radial stimuli may be summed to create a spiral in which the center of the spiral may move around the stimulus area 199.

FIG. 37 depicts the superposition of form and motion tests, herein called the spatial distractor tasks test, to assess the combination of visual motion and visual form. The subject 192 may control the position and movement of cursor 1050 to match that of the target, while form, motion, or other basic stimuli are combined with brief visual or auditory distracters to interfere with the task.

The starting phase of the spatial distractor tasks test 1380 may include the superimposed darker-contrast rectangles 1332 and lighter-contrast rectangles 1334 from the starting phase of the visual form discrimination test 1330 in FIG. 31 together with relatively higher contrast level darker dots 1312 and relatively lower contrast level lighter dots 1314 within the active stimulus radial segment 1310 from the starting phase of the dynamic contrast test 1300 in FIG. 25.

The number of darker-contrast rectangles 1332 and lighter-contrast rectangles 1334 in the starting phase of the spatial distractor tasks test 1380 may be fewer or more than the number of the equivalent elements of the starting phase of the other tests 1330. The number of relatively higher contrast level darker dots 1312 and relatively higher contrast level lighter dots 1314 within the active stimulus radial segment 1310 may be fewer or more than the number of the equivalent structures of in the starting phase of the dynamic contrast test 1300. Hence, both patterns may be shown with higher or lower cue element density than previously with the starting phase of other tests 1330 or, for another example, the starting dynamic contrast of the test 1300. This apportionment of cue elements may depend on the subject's performance on other tests or on other factors relevant to that subject's assessment.

Additionally, the darker rectangles 1332 and lighter rectangles 1334 in the starting phase of the spatial distractor tasks test 1380 have distinction levels set between the previously established threshold for distinctiveness, for example, as derived from the termination phase of the dynamic contrast discrimination test 1348 of FIG. 33. As described in great detail in the detailed description of the starting phase of the visual form discrimination test 1330, the darker rectangles 1332 and lighter rectangles 1334 may fade in and out in the neutral background stimulus area 1308 while moving to random new positions.

Additionally, relatively darker dots 1312 and relatively lighter dots 1314 within the active stimulus radial segment 1310 in the starting phase of the spatial distractor tasks test 1380 have contrast levels set between two confidence intervals below and above the established threshold for coherence from the termination phase of the dynamic contrast test 1322 in FIG. 27. It should be noted, that wherein this disclosure makes specific reference to performance of a dynamic contrast test, further embodiments may likewise perform a visual saliency test in a manner as described. It should be further noted, that wherein this disclosure, specific references are made to determination of contrast threshold, further embodiments of the present disclosure may employ the methodology to also determine additional coherence thresholds without departing from the scope of the present disclosure. Furthermore, these coherence thresholds may be employed in a similar manner. For example, some embodiments of the present disclosure may be configured to determine one or more coherence thresholds for various visual factors, including but not limited to, a brightness competency threshold, a contrast competency threshold, a background luminance competency threshold, and a frequency composition competency threshold. As described in great detail in the detailed description of the starting phase of the visual form discrimination test 1330, relatively higher contrast level darker dots 1312 and relatively lower contrast level lighter dots 1314 within the active stimulus radial segment 1310 may fade in and out in the neutral-contrast background stimulus area 1308 with randomly assigned life time periods that are chosen within a timed interval.

Further, the active stimulus radial segment 1310 may undergo the same sequence of settings and conditions outlined by the algorithm of the stimulus generator 450 as described in great detail in the starting phase of the visual form discrimination test 1330. Meanwhile, auditory, tactile, or visual distracters or other basic stimuli may interfere with the task, which may be associated with dual task interference. Further, dual task interference may require the subject to align one target area on top of another target area. Further, the subject may need to utilize two functions of its brain, which may cause interference amongst those brain functions.

FIG. 38 illustrates the intermediate phase of the spatial distractor tasks test 1390, a phase during which the focus of expansion 1352 moves with varying movements of motion coherence, location, direction, and speed outlined by the detailed description of the intermediate phase of the visual motion discrimination test 1360 in FIG. 35. The variations with the focus of expansion 1352 may be superimposed with active stimulus radial segment 1310 described in detail in the starting phase of the spatial distractor tasks test 1380 of FIG. 37. This superimposition of tasks may test the subject's cognitive processing ability while the subject 192 must utilize two functions of its brain, wherein the functions may interfere with each other.

In order to ensure that the subject 192 understands the complexity of the superimposed test iteration present in the intermediate phase of the spatial distractor tasks test 1390, the first continuous movement may be performed at two confidence intervals above the threshold established in termination phase of the dynamic contrast module test 1322 and two confidence intervals below the threshold established in the termination phase of the dynamic contrast discrimination test 1348. Subsequently, the continuous movement may be performed at two confidence intervals above the threshold established in termination phase of the dynamic contrast module test 1322 and two confidence intervals below the threshold established in the termination phase of the dynamic contrast discrimination test 1348.

The subject's control and movement of a subject manipulandum 402 may be implemented to track to the form target and the motion target onto the subject display 198 with the use of a cursor 1050. The form target and the motion target locations may be separated by a predetermined separation distance within the range of one-hundred fifty degrees and two-hundred ten degrees.

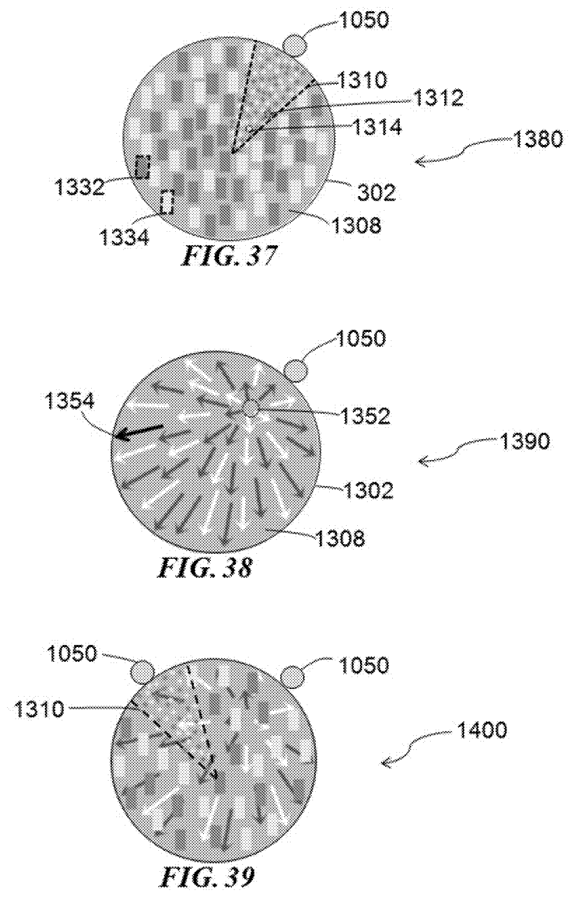

The subject 192 may use the cursor 1050 to track a form target, which includes the form changes of the darker-contrast rectangles 1332 and lighter-contrast rectangles 1334. The subject 192 may use the cursor 1050 to track motion of motion target, which includes the relatively higher contrast level darker dots 1312 and relatively lower contrast level lighter dots 1314. Further, the cursor 1050 may also be implemented to track the motion and to track the form in the respective tests of FIGS. 39, 40, and 41 as outlined in greater detail in the accompanying descriptions of those respective figures.

After a pre-selected or contextually derived time limit, the two stimuli of motion and form shift places in the paradigm and the subject 192 may be instructed to shift tasks.

FIG. 39 represents the left-up form target and right-up motion target of the visual motion and visual form attention test 1400. Both the patterns of darker-contrast rectangles 1332 and lighter-contrast rectangles 1334 and relatively higher contrast level darker dots 1312 and relatively lower contrast level lighter dots 1314 within the active stimulus radial segment 1310 may be superimposed during phase 1400.

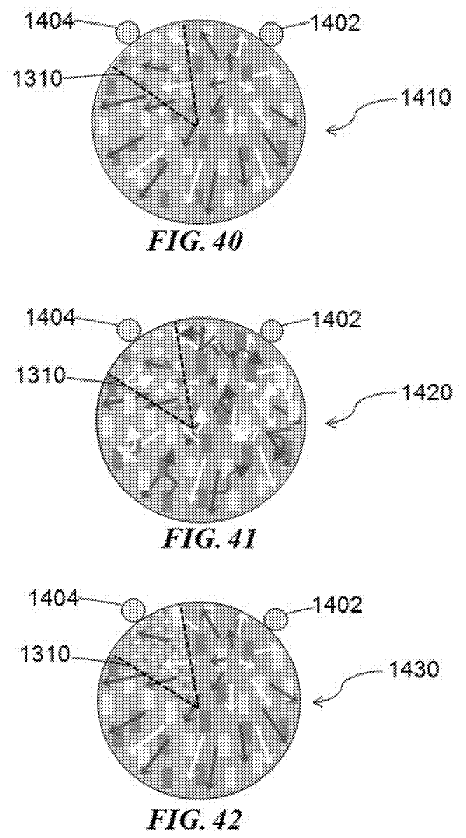

FIG. 40 displays the left-up form, low-distinct target and right-up motion, high-coherence target of the visual motion and visual form attention test 1410. Both the patterns of darker-contrast rectangles 1332 and lighter-contrast rectangles 1334 and relatively higher contrast level darker dots 1312 and relatively lower contrast level lighter dots 1314 within the active stimulus radial segment 1310 may be superimposed during the phase of the left-up form, low-distinct target and right-up motion, high-coherence target of the visual motion and visual form attention test 1410.

FIG. 41 shows the left-up form, high-distinct target and right-up motion, low-coherence target of the visual motion and visual form attention test 1420. Both the patterns of darker-contrast rectangles 1332 and lighter-contrast rectangles 1334 and relatively higher contrast level darker dots 1312 and relatively lower contrast level lighter dots 1314 within the active stimulus radial segment 1310 may be superimposed during the phase of the left-up form, high-distinct target and right-up motion, low-coherence target of the visual motion and visual form attention test 1420.

FIG. 42 portrays the left-up form, high-distinct target and right-up motion, high-coherence target of the visual motion and visual form attention test 1430. Both the patterns of darker-contrast rectangles 1332 and lighter-contrast rectangles 1334 and relatively higher contrast level darker dots 1312 and relatively lower contrast level lighter dots 1314 within the active stimulus radial segment 1310 may be superimposed during the phase of the left-up form, high-distinct target and right-up motion, high-coherence target of the visual motion and visual form attention test 1330.

Further, the spatial distractor tasks testing of the subject matter regarding FIGS. 37, 38, 39, 40, 41, and 42, may be added to any test of the present disclosure. The radial optic flow stimulus may be the substrate for the spatial distractor tasks testing; however any other functional assessment test may be associated with the stimulus for the substrate of the spatial distractor tasks testing. The present disclosure describes a subject 192 that is performing a spatial discrimination task and may position the cursor 1050, which may be any of the previously described or illustrated subject interface responses devices, at the location on the stimulus area 199 where the subject 192 sees a high saliency wedge within the stimulus area 199. The present disclosure may superimpose the intermittent addition of an alternative, high saliency cue somewhere else, such that the subject 192 may transiently shift attention to that distractor so that the distractor is not task relevant and also not to degrade the target following in the main task. The distractor may include, but is not limited to, a wedge of unique stimulus elements flashing for one to three seconds at a position far from the target wedge, an area of unique elements flashing on for one to three seconds at a position far from the target edge, or the transient displacement of the cursor 1050 to some place other than that specified by the subject 192.

Further, the spatial distractor tasks testing of the subject matter regarding FIGS. 37, 38, 39, 40, 41, and 42, may be associated with spatial memory testing, in which the spatial memory of a subject 192 may be used to augment the subject's response sensitivity in any of the main tasks, which may include, but it not limited to, form, motion, and words. In these main tasks, the target wedge may transiently flash to some high saliency cue, which may include, but it not limited to one hundred percent saliency of the target cue, or all white, or all black, and then may revert to its near threshold saliency and makes a stereotyped movement or selected number of movements. After repeated exposures, the subject 192 may implicitly, that is without being told, acquire knowledge of the flashes' meaning. The subject 192 may use that information to enhance the ability to follow the target stimulus through that spatial sequence; for instance, the subject 192 may further use movement as a stimulus for learning a sequence of movements. Further, spatial memory testing may include, but is not limited to sequence memory or location memory. Further, spatial memory testing may be a combination of testing associated with sequence memory and location memory.

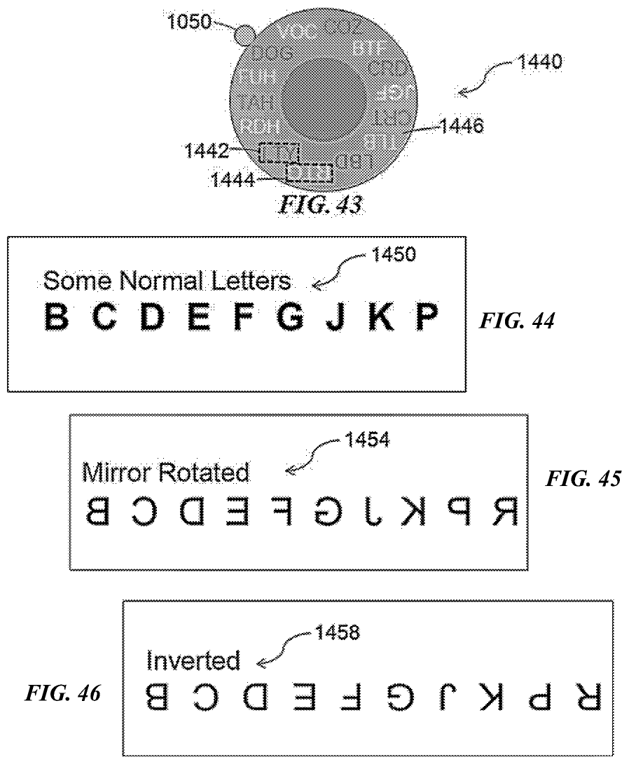

FIG. 43 displays the starting phase of the word identification latency module 1440, during which equal numbers of alternating black-colored letter sets 1442 and white-colored letter sets 1444 may be presented in a fixed sequence around the edge of circular, stimulus area 1446. The three letters words may be distributed in the background, which may comprise a cluster of other three letter sets and also a real word that defines a target. Further, a word may be associated with correct letters that may be imbedded in a stimulus ring with three letter figures made of non-letters.

The three letters for the alternating black-colored letter sets 1442 and white-colored letter sets 1444 may fall into the following categories of: 1) target word, 2) legal-non-words, 3) illegal non-words, 4) flipped illegal non-words, and 5) flipped and rotated non-word. The three letters may be in different orientations or may utilize false fonts as further outlined in FIGS. 44, 45, and 46.

Font, size, and position of the black-colored letter sets 1442 and white-colored letter sets 1444 may be determined by the pre-sets from the starting phase of the visual motion discrimination test 1350 and the starting phase of the visual form discrimination test 1330. The contrast of the letters may be set at being two confidence intervals above the subject's contrast threshold obtained in the termination phase of the visual motion discrimination test 1370.

Herein, the words vs non-words task may be made more difficult in a variety of ways including. Difficulty variety may include, but not be limited to: 1) the superimposition of random dots on the entire stimulus area with greater numbers or sizes of dots increasing task difficulty, 2) the varying the relative position of letters in the words and non-word foils so as to crowd or separate, tilt, or misalign the letters, with greater such effects or combinations of effects increasing task difficulty, and/or 3) the selection of legal non-word foils (e.g., having the regular consonant-vowel-consonant structure of words) versus illegal non-words (e.g., consonant-consonant-consonant structure not seen in words), wherein the legally structured non-words may be more difficult to reject as candidate target words than illegally structured non-words.

FIG. 44 shows normal letters orientation 1450, which may be applied towards the three letters that were described previously in the starting phase of the letter identification latency module 1440 of FIG. 43.

FIG. 45 shows mirror rotated letters orientation 1454, which may be applied towards the three letters that were described previously in the starting phase of the letter identification latency module 1440 of FIG. 43.

FIG. 46 shows inverted letters orientation 1458, which may be applied towards the three letters that were described previously in the starting phase of the letter identification latency module 1440 of FIG. 43.

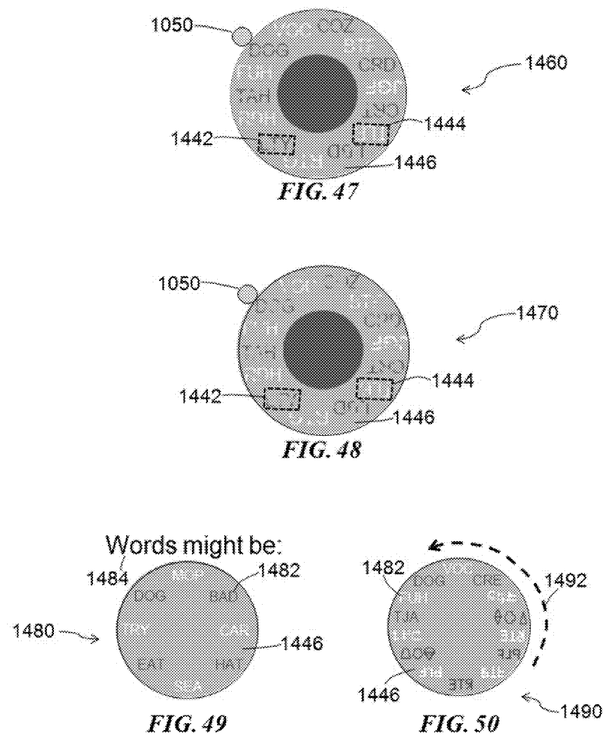

FIG. 47 shows the intermediate phase of the letter identification latency module 1460, during which the three letters of the black-colored letter sets 1442 and white-colored letter sets 1444, which may be within the circular stimulus area 1446, may be partially obscured to reduce their saliency and to establish the cursor tracking response function. During the start of the test paradigm of the intermediate phase of the letter identification latency module 1460, the subject 192 may be presented with the highest level of letter continuity. A plurality of the item stimulus may set drift around the stimulus area 199, which may be a ring, in unison. The subject 192 may move the cursor 1050 to the real word and follow it for a predetermined time period or a predetermined extent as angular degrees of drift. The score may be derived from the time it takes the subject 192 to register the location of the real word that may be captured and tracked.

Subsequently, word continuity may be continually and algorithmically disrupted by the superimposition of background color line segments that occlude a set percentage of the length of the line segments forming the characters in the display. The subject 192 may be asked to follow the letter sets using the cursor 1050 during the continuous movement of the letter sets around the around the edge of circular stimulus area 1446.

The letter sets in the array may drift in unison around the display circle or may emerge and fade to take-up new positions on the screen with a full field random cycle length in a settable range, which may be typically thirty-six to one-hundred eight frames at seventy-two hertz with emergence and fading each occurring over three frames. The position and continuity of the letter sets may be subjected to the algorithmic control of the stimulus generator 450. Each position shift may trigger the transition of all character sets to other specific example of each set type in the corresponding relative positions.

In an alternate embodiment of the intermediate phase of the letter identification latency module 1460, a word may be made of correct letters imbedded within the stimulus area 199, which may be a ring, with other similar length, correct letter, non-words. All of the three-letter items may drift around the ring in unison. The subject 192 may move the cursor 1050 to the real word and follow it for a predetermined time period or a predetermined angular degrees of drift. The score may be derived from the time it takes the subject 192 to register the location of the real word that may be captured and tracked.

In yet another embodiment of the intermediate phase of the letter identification latency module 1460, correct letter words may be imbedded in the stimulus area 199, which may be a ring, with other similar length, correct letter, non-words. All of the three-letter items my drift around the ring in unison. The content of the ring, which may refer to its real words and non-words, my change regularly as the content drifts so there is always a wedge, which may be a ring segment, containing real words and the remainder of the ring contains non-words. Further, as the subject 192 moves the cursor 1050 to the real word and follows it for some predetermined time period or a predetermined angular degrees of drift, the saliency of all of the letters of the words and non-words may be slowly decreased. The saliency may be decreased either by crossing-out parts of all of the letters with a background colored set of thin lines, or by rotating the individual letters, or by covering the ring with flickering letter-colored dots. The subject 192 may continue to find the real words as algorithmic adjusting of the saliency determines that subject's threshold saliency. The score is derived from the saliency level as described for the other tests of the present disclosure.

FIG. 48 shows the termination phase of the letter identification latency module 1470, during which an approximate threshold may be defined. There remains continuous movement of the target character set and subject tracking during continuous varying of the continuity and exchange of all character sets across cycles towards the end of intermediate phase of the letter identification latency module 1460.

Later, during the termination phase of the letter identification latency module 1470, while in discontinuous movement, the target segment may fade to the background parameters and then may emerge at a new location where it may undergo increasing continuity until the subject's cursor may enter the target segment area. Immediately thereafter, there may be an instantaneous bright flash and beep. Subsequent iterations of this trial may yield a refined threshold.

FIG. 49 illustrates the starting phase of the verbal memory module 1480. This test paradigm may present a series of words 1482 in a list to be memorized. The sample consists of a series of words 1482 that may be arranged around the edge of the stimulus area 199 and headed by the label “Words might be” 1484. The sample words are positioned at selected locations with selected light and dark luminance. During the starting phase of the verbal memory module 1480, the subject 192 may be presented a predetermined series of short words, each with a predetermined number of letters in a set sequence.

FIG. 50 displays the intermediate phase of the verbal memory module 1490. The subject 192 may track the target word in the series of words 1482, starting form low saliency and successively becoming more salient, via the presentation of sample and match across contrast stimuli 1492. A particular word in a series of words 1482 may be presented one-at-a-time along with words not on the list. In other words, in this series of stimuli, the word target may be either sample words or not.

During the intermediate phase of the verbal memory module 1490, the subject 192 may be first shown a series of ten high contrast black or white words for a pre-set adjustable time period, which may be for five seconds. The subject 192 may then be shown a series of the same type of stimuli that may have been used in the starting phase of the letter identification latency module 1440 as was shown in FIG. 43. The presentation of sample and match across contrast stimuli 1492, which may be implemented in the intermediate phase of the verbal memory module 1490, may be the same fade-jump-emerge contrast modulation sequence that may have been used in the intermediate phase of the letter identification latency module 1460.

In an alternate embodiment of the intermediate phase of the verbal memory module 1490, the target word from a predetermined ordered list may be presented at very low saliency after each presentation of a predetermined series of short words. That target word from a predetermined ordered list may drift around the stimulus ring imbedded in with other drifting three-letter sets that are not words. While the subject 192 remains off target, the saliency of the word and the three letter non-words may slowly increase until the word is recognizable as the only word on the screen. The subject 192 may move the cursor 1050, which may be any of the direct or remote contact subject interface response devices, to the target word and follow it for some predetermined time period or a predetermined degrees of angular movement to register correct acquisition. When the subject 192 has correctly identified the target word, the score for that trial is recorded as the current saliency level. Then, the next word from the list may be imbedded in a new set of three letter non-words at very low saliency and the task continues. The cycle of first viewing the list presentation of these predetermined list of words and then testing on finding the words at the lowest saliency possible may be repeated three times. Scoring of the test may include the number of words correctly acquired, the saliency level at which they were acquired, and the slope of the average saliency levels across the three repetitions of the task.

In yet another embodiment of the intermediate phase of the verbal memory module 1490, only one target word may be implemented. In this exemplary embodiment, after the saliency score is calculated, the number of target words may be slowly increased to repeatedly derive that subject's saliency threshold as the word list length increases. If one knows the word one is looking for, then it may be relatively easy to find it; however, the degree of difficulty may increase with an increase in the number of words. Each subject 192 may have a function of saliency versus list length and that may be a measure of verbal memory's ability to enhance word recognition.

An alternate embodiment of the intermediate phase of the verbal memory module 1490, may include, but is not limited to, a ring with only correct letter words. As the subject 192 correctly follows the initially single word around the ring, another word will be added and the subject 192 may shift to following the new word. Throughout the test, new words may be added and may be monitored for how long it takes the subject 192 to identify and shift to the new word most recently added to the subject display 198. Scoring may be accomplished by measuring the new word identification latency, as a function of the total number of words in the display during that response.

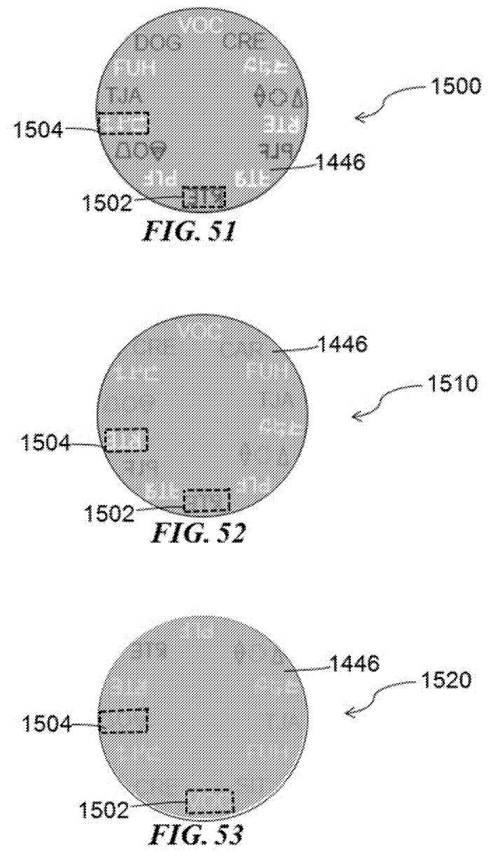

The responses to the stimuli from the intermediate phase of the verbal memory module 1490 may be used to establish response dynamics in the stimulus contrast domain and the kinematics domain. During the intermediate phase of the verbal memory module 1490, the target orientation may be placed towards the left or towards the right of the stimulus area 199, and may be either high, moderate, or low contrast. FIGS. 51, 52, and 53 show the various placement configurations and contrast conditions that may be implemented during the intermediate phase of the verbal memory module 1490.

With reference to FIGS. 51, 52, and 53, equal numbers of alternating black-colored symbol sets 1502 and white-colored symbol sets 1504 may be presented in a fixed sequence around the edge of circular stimulus area 1446. The three letters symbol sets may be distributed in the background that may comprise a cluster of other three letter symbol sets and also a real word that defines the target.

The three symbols for the alternating black-colored symbol sets 1502 and white-colored symbol sets 1504 may include, but are not limited to, symbols, target words, legal-non-words, illegal non-words, flipped illegal non-words, flipped and rotated non-words. Further, the three letters symbol sets may be in any orientation. Further, the font, size, and position of the black-colored symbol sets 1502 and white-colored letter symbol sets 1504 may be determined by the pre-sets from the starting phase of the visual motion discrimination test 1350 and the starting phase of the visual form discrimination test 1330. The contrast of the black-colored symbol sets 1502 and white-colored letter symbol sets 1504 may be set at being two confidence intervals above the subject's contrast threshold obtained in the termination phase of the visual motion discrimination test 1370.

More particularly, FIG. 51 illustrates the left-up target orientation with black-colored symbol sets 1502 and white-colored symbol sets 1504 in high contrast. FIG. 52 shows the right-up target orientation with black-colored symbol sets 1502 and white-colored symbol sets 1504 in moderate contrast. FIG. 53 displays the right-down target orientation with black-colored symbol sets 1502 and white-colored symbol sets 1504 in low contrast.

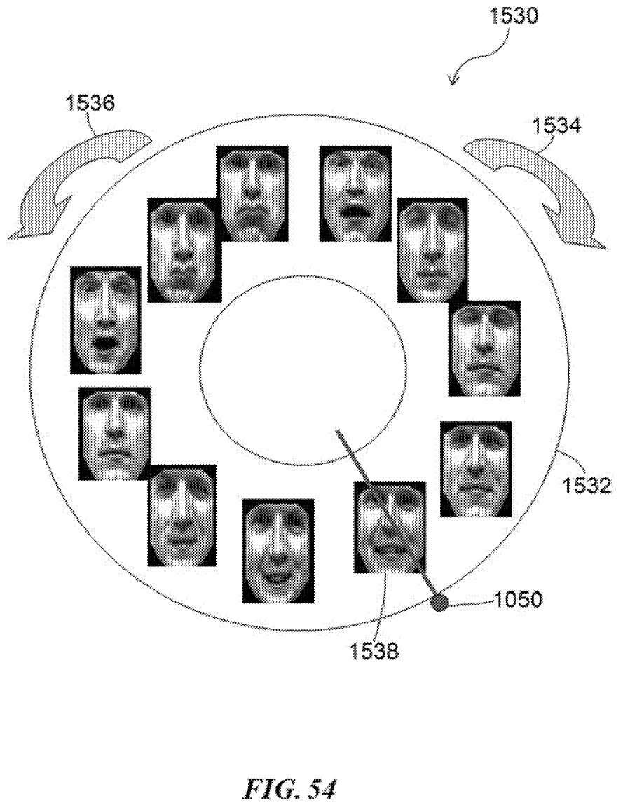

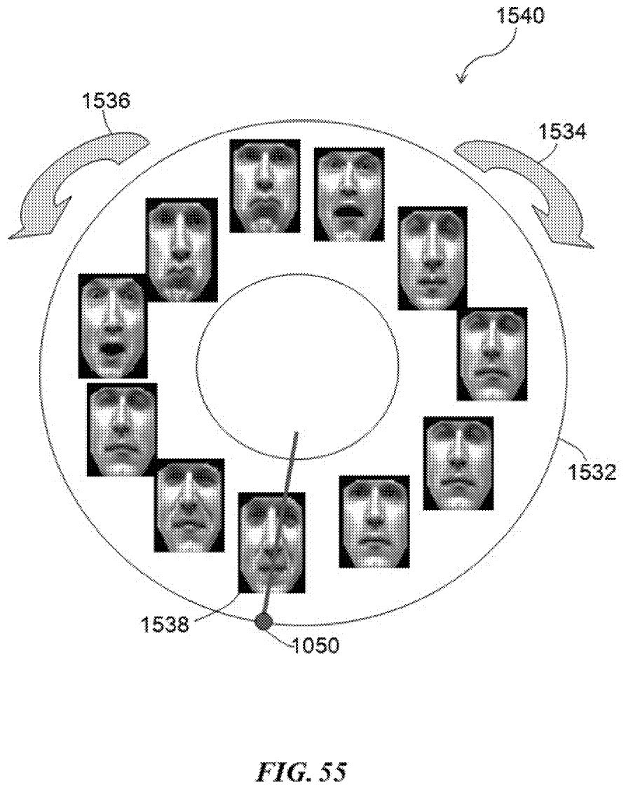

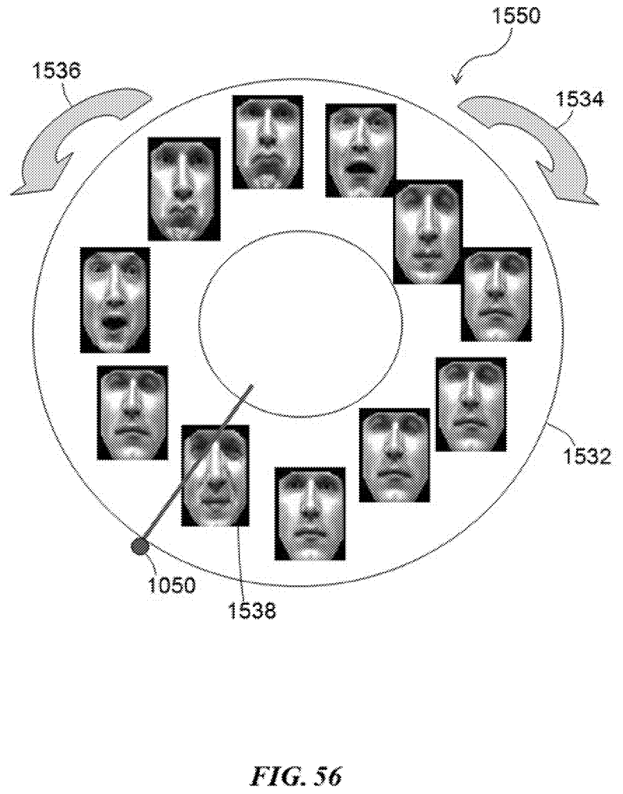

With reference to FIGS. 54, 55, and 56, facial emotion sensitivity tests may be presented to the subject 192. More particularly, FIG. 54 shows a low difficulty facial emotion sensitivity test 1530, FIG. 55 shows a moderate difficulty facial emotion sensitivity test 1540, and FIG. 56 shows a high difficulty facial emotion sensitivity test 1550, for any of which a display of faces 1532 may be presented to the subject 192. A plurality of faces, may be all of the same person or may be a pseudo-person composite of other faces.

Subsequently, the affective emotion may be modulated, such as from grimace or frown to a wide-eyed or smile emotion. There may be a gradient of emotion expressions distributed across the faces, from happy faces at one point to sad faces one hundred eighty degrees from that point. The subject 192 may locate and may track the happiest face or the saddest face. The subject 192 may be asked to use the subject manipulandum 1402 to point to the happier faces as the differences between the happier and sadder faces may be narrowed with good performance or widened with poor performance. The subject 192 may demonstrate a minimal difference in affective expression required for their identifying the most positive or happy expression. The subject 192 may use the rotatory manipulandum 414 to rotate and to align the cursor 1050 to the happiest face 1538 as the range from sad to happy is increased, thereby making task easier, or decreased, thereby making task harder. The subject 192 may rotate the rotatory manipulandum 414 in a clockwise rotation 1534 or in a counterclockwise rotation 1536.

The algorithm associated with the present disclosure may alter the range of faces, which may be from very happy to very sad, very calm to very anxious, very passive to very aggressive. The algorithm associated with the present disclosure may alter the range of faces, which may vary continually along the aforementioned continua, i.e., from slightly happy to slightly sad. The mid-point may be from happy to neutral, or in an alternative embodiment may be from neutral to sad. Further, the algorithm associated with the present disclosure may be easy or difficult. Further, the subject's score may be a reflection of the minimal range, which may be of greatest difficulty, at which the subject 192 may accurately locate and track the target, i.e., happiest or saddest or most neutral face.

The low difficulty facial emotion sensitivity test 1530, moderate difficulty facial emotion sensitivity test 1540, and high difficulty facial emotion sensitivity test 1550 differ in the level of difficulty within each test. Further, the low difficulty facial emotion sensitivity test 1530, moderate difficulty facial emotion sensitivity test 1540, and high difficulty facial emotion sensitivity test 1550 may help determine the test subject's perceptual threshold range scored relative to a normal range derived from comparison subject groups. Facial gender, age, and identity may be randomly shifted during intervals of the test session. Future known equivalents of the low difficulty facial emotion sensitivity test 1530, moderate difficulty facial emotion sensitivity test 1540, and high difficulty facial emotion sensitivity test 1550 may use only one gender, age, etc. facial identity group or can use alternative target, which may include, but is not limited to, the saddest face.

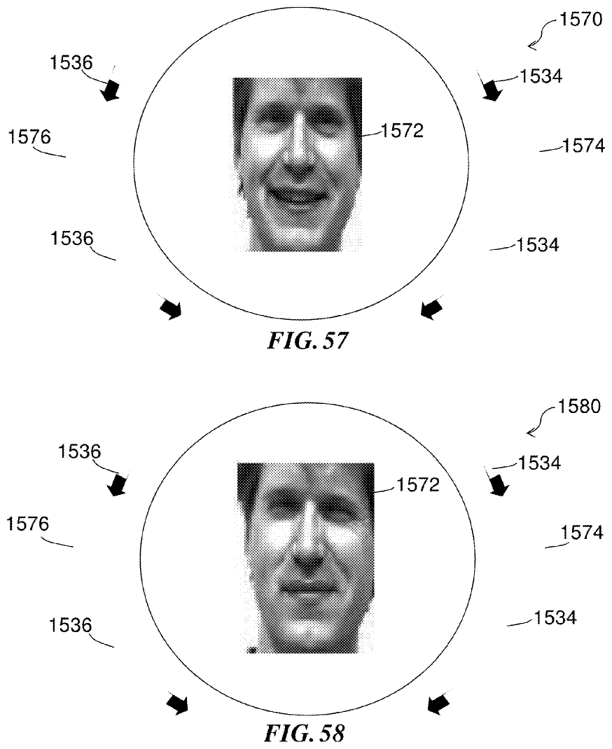

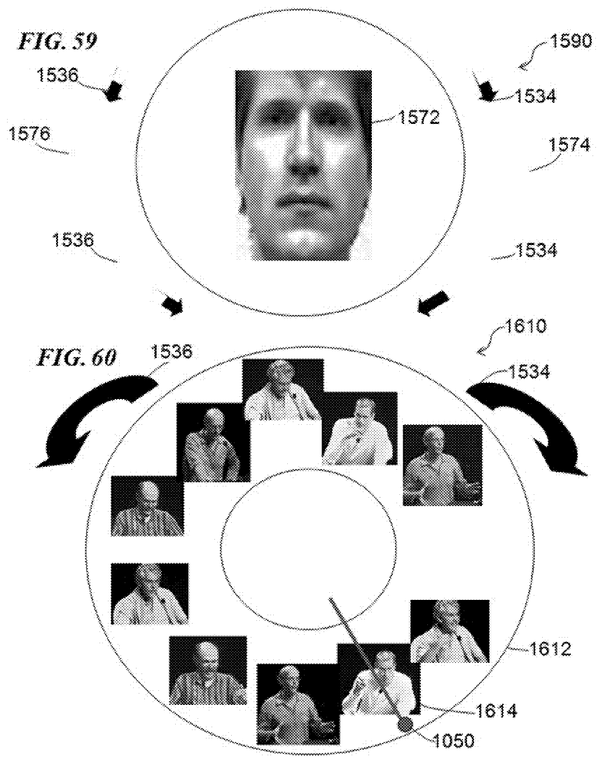

With reference to FIGS. 57, 58, and 59, facial emotion nulling tests may be presented to the subject 192. More particularly, FIG. 57 shows a low difficulty facial emotion nulling test 1570, FIG. 58 shows a moderate difficulty facial emotion nulling test 1580, and FIG. 59 shows a high difficulty facial emotion nulling test 1590, for any of which a display of a particular facial expression 1572 is presented to the subject 192.

During either the low difficulty facial emotion nulling test 1570, moderate difficulty facial emotion nulling test 1580, or a high difficulty facial emotion nulling test 1590, a single image of a same gender face is presented and the system varies the affective expression of the face from a sadder to a happier expression and vice-a-versa.

The emotional expression of the single face may be varied as described in the low difficulty facial emotion sensitivity test 1530, moderate difficulty facial emotion sensitivity test 1540, and high difficulty facial emotion sensitivity test 1550. During either the low difficulty facial emotion nulling test 1570, moderate difficulty facial emotion nulling test 1580, or a high difficulty facial emotion nulling test 1590, the subject 192 may uses the subject manipulandum 402 to make the face appear neutral, which may refer to being neither happy nor sad. The subject 192 may be asked to rotate the rotary manipulandum 414 with counter-clockwise rotation 1534, thereby making the expression sadder with the use of the turn to make sadder feature 1576, or with clockwise rotation, thereby making the expression happier with the use of the turn to make happier feature 1574.

The goal of the subject 192 may be to continue to rotate the rotary manipulandum 414 to make the expression neutral as the present disclosure makes sustained changes in the affective expression of the facial display. The subject 192 may use the rotatory manipulandum 414 to morphologically transform facial expression across the spectrum from sadder, which may be through repeated counterclockwise rotation 1536, to happier, which may be through repeated clockwise rotation 1534, to keep the facial expression neutral.

The algorithm of the present disclosure may continually shift the emotional content of the facial expression and the subject 192 may have to change it back toward neutral. Such a test may be associated with being a nulling task, wherein only the parameter is changed, and the subject 192 has to perceive the direction and magnitude of the change and set it back to where it was. The scoring may reflect the magnitude of change required to trigger the subject's response, the point called neutral from happy and the point called neutral from sad.

The low difficulty facial emotion nulling test 1570, moderate difficulty facial emotion nulling test 1580, or a high difficulty facial emotion nulling test 1590 each may be sixty to one-hundred eighty seconds in duration. The system repeatedly may drift the facial expression to a sadder or to a happier condition as the subject 192 may try to null that effect and may try maintain a neutral expression on the display. The system may use an adaptive staircase protocol to determine the smallest perturbation of facial expression that may provoke an appropriate counter-response from the test subject 192 as a facial expression perceptual threshold, which may be scored relative to normal range identifiable by others in the comparison subject group.

Facial gender, age, and identity may be randomly shifted during intervals of the test session. Future known equivalents of the low difficulty facial emotion nulling test 1570, moderate difficulty facial emotion nulling test 1580, or a high difficulty facial emotion nulling test 1590 may use only one gender, age, etc.

Further, the low difficulty facial emotion nulling test 1570, moderate difficulty facial emotion nulling test 1580, or a high difficulty facial emotion nulling test 1590 each differ in the level of difficulty within each test.

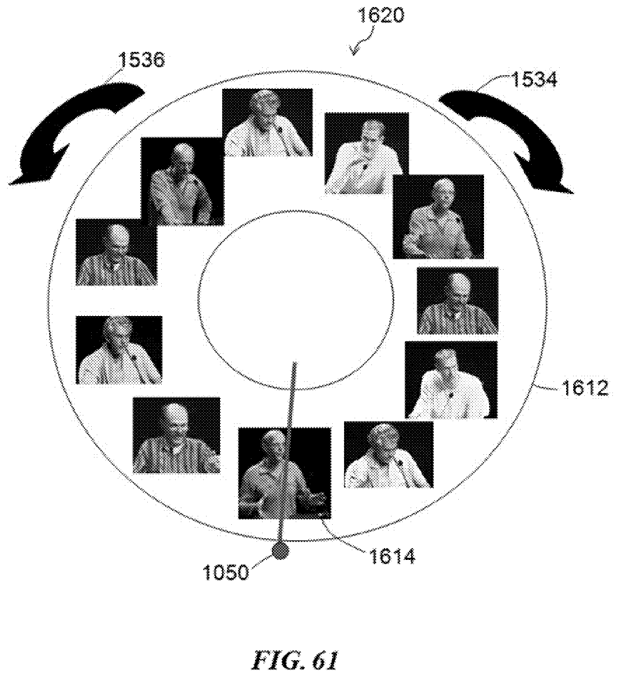

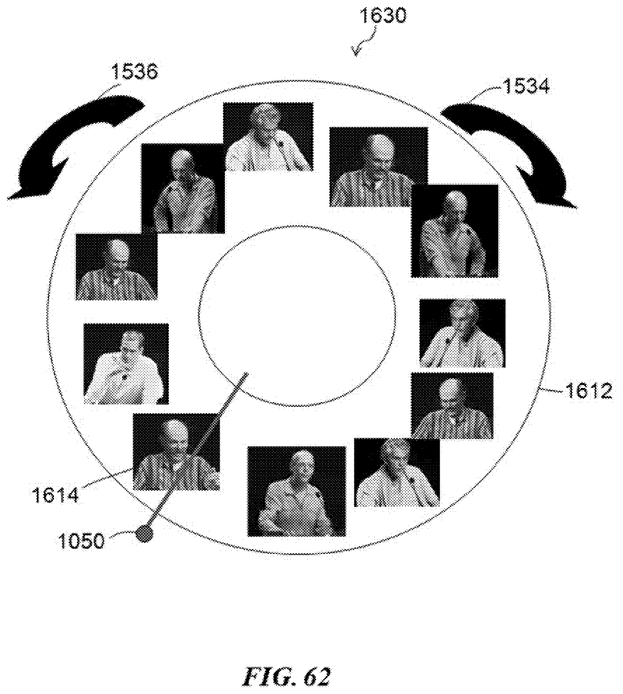

With reference to FIGS. 60, 61, and 62, social cues sensitivity tests may be presented to the subject 192. More particularly, FIG. 60 illustrates the low difficulty social cues sensitivity test 1610, FIG. 61 illustrates the moderate difficulty social cues sensitivity test 1620, and FIG. 62 illustrates the high difficulty social cues sensitivity test 1630, for each of which a display of varying aggressiveness levels 1612 may be presented to the subject.

In one embodiment, the display of varying aggressiveness levels 1612 may show a number of whole body images of different persons. The subject 192 may use the rotatory manipulandum 414 to align the cursor 1050 to the image of the person being most aggressive, herein called the most aggressive person 1614. The subject 192 may rotate the rotatory manipulandum 414 in a clockwise rotation 1534 or in a counterclockwise rotation 1536 to indicate the most aggressive person 1614 on the display of varying aggressiveness levels 1612. As the range from submissive to aggressive is increased, thereby making the task easier, or decreased, thereby making the task harder, the perceptual threshold of the subject 192 relative to a normal range may be characterized in comparison.

In an alternate embodiment, a variety of different body positional attributes may be displayed. For example, the body positional attribute may be associated with the most/least worried or the most/least frightened or the most/least leadership ability or the most/least assertive. The body positional attribute of least worried may be associated with, but is not limited to, smiling, titled head and shoulders, and hands at the side. The body positional attribute of most worried may be associated with, but is not limited to, pursed-lips, slouched head and shoulders, and hands tightly clasped in front of the lower face. The body positional attribute of most frightened may be associated with, but is not limited to, eyes bulging, limbs flexed, and jerky movements. The body positional attribute of least frightened may be associated with, but is not limited to, smiling, upright, and slow movements.

Person gender, age, ethnic group, and other identifying facial characteristics may be randomly shifted during intervals of the test session for any or all of the low difficulty social cues sensitivity test 1610, the moderate difficulty social cues sensitivity test 1620, or the high difficulty social cues sensitivity test 1630. Future known equivalents of any or all of the low difficulty social cues sensitivity test 1610, the moderate difficulty social cues sensitivity test 1620, or the high difficulty social cues sensitivity test 1630 may use only one gender, age, etc. postural identity group or can use alternative target features, which may include, but is not limited to, the most submissive person.

Further, the low difficulty social cues sensitivity test 1610, the moderate difficulty social cues sensitivity test 1620, or the high difficulty social cues sensitivity test 1630 may also consider the interactions between the persons depicted in the display of varying aggressiveness levels 1612 such that the subject 192 indicates who may be the most likely to be leader of the group. The subject 192 may change the cursor 1050 to indicate who they see as the likely leader with differences between target leaders' traits and those of the person least likely to assume leadership are successively changed.

Further, the low difficulty social cues sensitivity test 1610, the moderate difficulty social cues sensitivity test 1620, or the high difficulty social cues sensitivity test 1630 each differ in the level of difficulty within each test.

In an alternative embodiment of social perception domain testing, nulling adjustments may be evaluated in the social interactions nulling test, which may include, but is not limited to, a full body representation of two people standing side-by-side in an ongoing social interaction. One person may stand on the left side and another person may stand on the right side. One person may be a man, and the other person may be a woman; alternatively, both persons may be of the same sex. Further, one person may be of a particular ethnic background; another person may be of a different ethnic background; alternatively, both persons may be of the same ethnic background. During social interactions nulling testing, postures, facial expressions, and/or gestures may be distinctive among the two people; however, the two persons may not interact with words. The subject 192 may be instructed to adjust the left or right person to make one more dominant and the algorithm will change the balance, thereby making nulling adjustments.

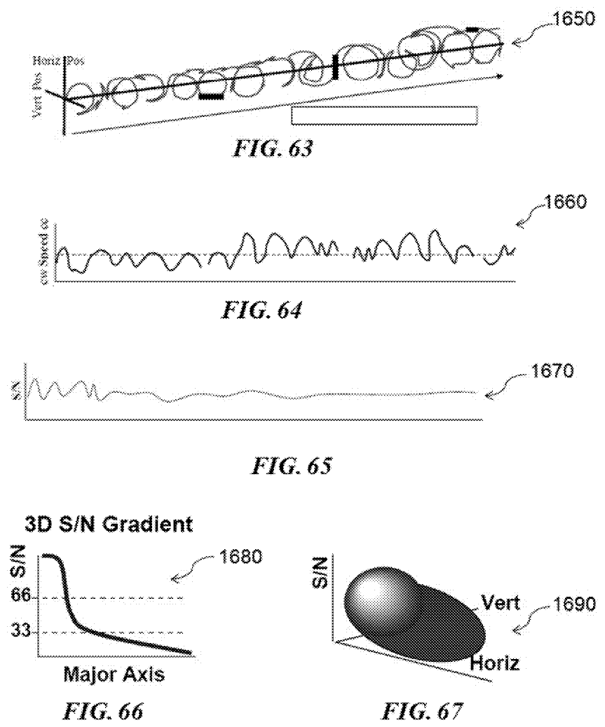

With reference to FIGS. 63, 64, and 65, typical target traces are presented, which may be, but are not limited to, sixty seconds traces. FIG. 63 shows an exemplary position trace 1650. FIG. 64 illustrates an exemplary speed trace 1660. FIG. 65 depicts an exemplary acceleration trace 1670.

The exemplary position trace 1650, the exemplary speed trace 1660, and the exemplary acceleration trace 1670 may show the target location, which may be driven in a tracking fashion by the stimulus generator 450 or in discontinuous fashion by jumping movements. Further, the exemplary position trace 1650, the exemplary speed trace 1660, and the exemplary acceleration trace 1670 may show initially, the highest signal-to-noise stimuli that may trigger the subject capture, which may refer to the positioning near the center of the highest signal-to-noise segment.

The exemplary tests of the present disclosure capture may be followed by irregular tracking movements with graded signal-to-noise fade-emerge cycles that may trigger capture cycles. Further, the exemplary tests of the present disclosure capture may include increasing, then decreasing, position and velocity error. During the exemplary tests of the present disclosure, escape, which may refer to gradually increasing error, may trigger either: 1) fixed-position re-emergence to trigger re-capture and then continuing movement, or 2) full-fading, jump to a new site, and re-emergence there until re-capture triggers new tracking movements. Further, uniformity of the distribution of capture position may be assisted by jumps and movement parameters may during signal-to-noise (S/N) fading cycles that may be based on subject error.

With reference to FIG. 66, an exemplary 3D S/N Gradient 1680, wherein S/N may refer to signal-to-noise ration, is presented. The exemplary 3D S/N Gradient 1680 may be representative of being across all stimulus domains. The exemplary tests of the present disclosure may be implemented to achieve a three-fold signal-to-noise gradient. More particularly, during the exemplary tests of the present disclosure, from the point furthest from the target in the stimulus area 199, there may be a gradual increase to one-third of the current peak signal-to-noise ratio at the edges of the target segment, which may be a thirty degrees segment. Further, another one-third signal-to-noise ratio increase may extend from the thirty degrees edges to a ten degrees segment in the stimulus area 199. The exemplary tests of the present disclosure may be structured such that the peak signal-to-noise should extend uniformly across the ten degrees segment, which may result in the hypothetical 3D S/N Gradient 1680.

With reference to FIG. 67 an exemplary S/N profile 1690 with respect to vertical and horizontal positions is presented. An exemplary S/N profile 1690 may be reflective of subject 192 response analyses that indicate the subject 192 may accurately track to yield reliable performance across all domains. Such reliable performance may be achieved via following of recommendations, which may be, but is not limited to:
i. The first stimulus cycles of each test of the present disclosure may be at low motion parameters and high signal-to-noise ratios so that the subject 192 may understand the task.
ii. Motor performance may be established by imposing a series of movement acceleration-deceleration cycles or direction reversal cycles in at least two of the four quadrants of the hypothetical S/N profile 1690.
iii. Subsequent cycles may include cue fading, which may result from decreasing the signal-to-noise ratio, such that when the cue escapes, the motion may slow in order to see whether the subject 192 may reduce the error distance. If the subject 192 catches-up, then the slower speed may become the new base speed. However, if error reduction does not occur, then the target slows down to a stop and the signal-to-noise ratio is increased until re-capture triggers the resumption of movement.
iv. There may be a jump to a new position near the current response position by slowly increasing the signal-to-noise ratio.
v. Repeated test cycles may be used to refine the impression of the signal-to-noise threshold and fastest speed and acceleration that the subject may accurately track to yield reliable performance across all conditions.

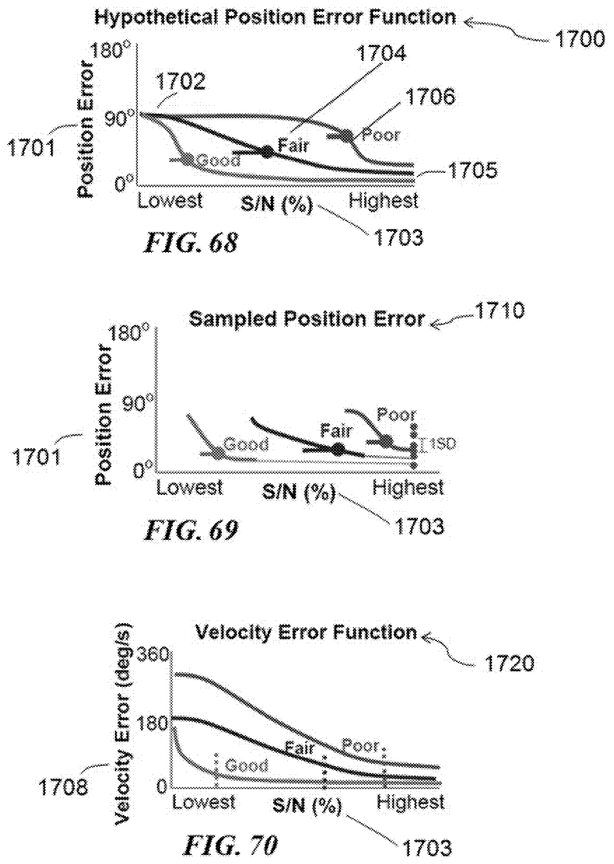

FIG. 68 shows an exemplary position error function profile 1700, which may be a plot of error by signal-to-noise to describe the performance of the subject 192. A graph of the position error axis 1701 versus the signal-to-noise percentage axis 1703 that may be present in the position error function profile 1700. The position error maximum 1702 and the position error minimum 1705 may be asymptotic projections, which may capture the best and the worst performance of the subject 192. The position error peak slope 1706 may be the mid point in the range of plus or minus five percent of the highest slope. The position error area 1704 under the curve of the position error function profile 1700 may describe the overall performance of the subject 192. Further, the position error function profile 1700 may be qualitatively grouped into profiles based on degree of differences, such as being good, fair, and poor.

FIG. 69 shows an exemplary sampled position error function profile 1710, which may be a plot of the position error axis 1701 versus the signal-to-noise percentage axis 1703, on a sampled basis. The exemplary sampled position error function profile 1710 may be based on a threshold and a variance measure from the tests of the present disclosure. For instance, in the visual motion discrimination test, which is further described in FIGS. 34, 35, and 36, the threshold is taken to be the signal-to-noise ratio under the point on the sampled position error function profile 1710 that is two position error significant digits back on along the sampled position error function profile 1710 curve. The present disclosure may utilize the range of the signal-to-noise covered by the two position error significant digit steps as a variance measures. The measures that may be implemented in the position error function profile 1700 and the sampled position error function profile 1710 may be sensitive to best performance, capture escape variability, and the local slope of the position error curve.

FIG. 70 displays an exemplary velocity error function profile 1720, which may be a plot of the velocity error axis 1708 versus the signal-to-noise percentage axis 1703. The velocity error function profile 1720 may show a representation of the difference between the stimulus and the response velocity.

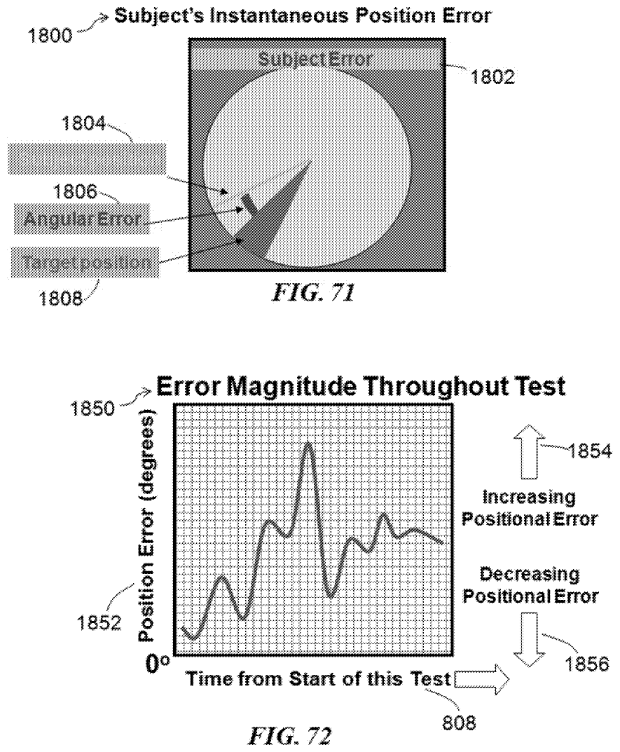

FIG. 71 portrays the instantaneous position error 1800 of the subject 192. The subject error 1802 may be a function of the subject position 1804, the angular error 1806, and the target position 1808. The subject error 1802 may be an error in the selection of the target on the stimulus area 199 by the subject 192. The subject position 1804 may be an error in the position of the target on the stimulus area 199 by the subject 192. The angular error 1806 may be an error in the angular position of the target on the stimulus area 199 by the subject 192.

FIG. 72 shows an exemplary output for graphical representation of the error magnitude throughout test 1850, which may be a plot of the position error in degrees 1852 versus the time from the start of this test 808. For illustration purposes, the exemplary output details a ten second intervals 806. However, the system may utilize greater or shorter time intervals. Further outputs that the system may output include detailing the error magnitude throughout test 1850, and the presence, or lack thereof, of increasing positional error 1854 with a higher value of time from the start of this test 808. Further outputs, may detail decreasing positional error 1854 with a lower value of time from the start of this test 808.

Further, the error associated with the error magnitude throughout test 1850 may peak at an escape event, during which a subject 192 may lose track of the target, but may decrease when the subject 192 re-captures the target to successively converge on subject's typical error margin. The error may be signed as being plus or minus one-hundred and eighty degrees relative to the direction of target movement, with the subject 192 being ahead or behind that movement.

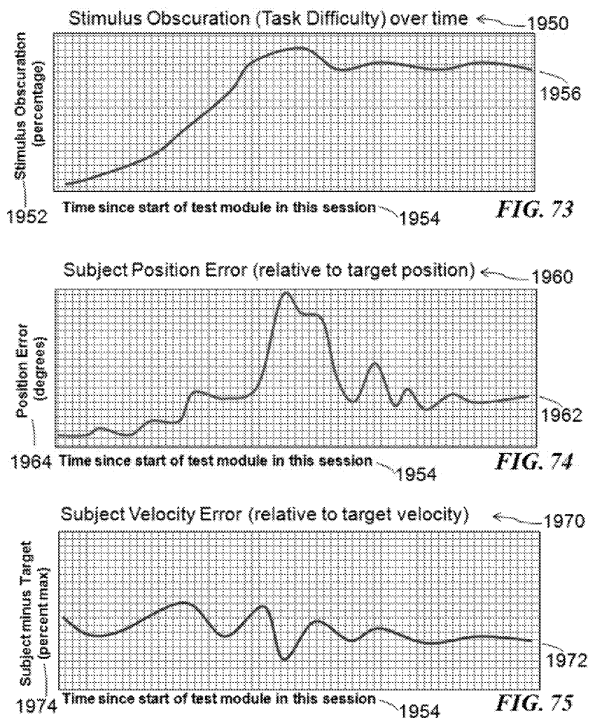

FIG. 73 depicts the stimulus obscuration over time 1950 output of the present disclosure. This output may illustrate the task difficulty over time. More particularly, the graph of stimulus obscuration over time 1956 may be a graph of percentage stimulus obscuration 1952 versus time since start of test module in this session 1954. Further, the time since start of test module in this session 1954 may be represented, but is not limited to, as being five seconds intervals.

FIG. 74 displays an exemplary output for the subject position error input relative to target position 1960. The subject position error input relative to target position 1960 may be a graph of subject position error over time 1962, which may be represented as a graph of position error in degrees 1964 versus time since start of test module in this session 1954. Further, the time since start of test module in this session 1954 may be represented, but is not limited to, as being five seconds intervals.

FIG. 75 depicts an exemplary output of the present disclosure for subject velocity error relative to target velocity 1970. More particularly, the graph of subject velocity error relative to target velocity 1972 may be graphically represented as subject minus target as percent maximum 1974 versus time since start of test module in this session 1954. Further, the time since start of test module in this session 1954 may be represented as subject minus target as percent maximum versus but is not limited to, as being five seconds intervals.

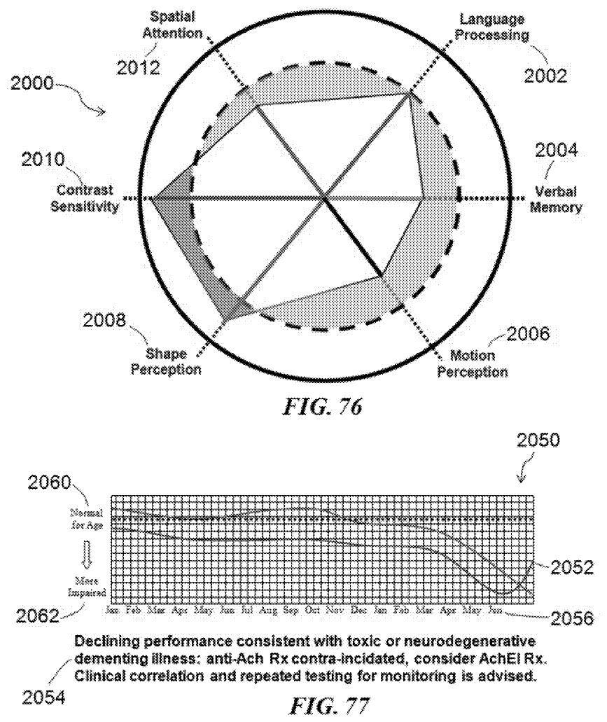

FIG. 76 shows an exemplary results summary 2000 output that may be displayed by the present disclosure via a graphical user interface. The results summary may include, but is not limited to, a representation of the quantitative assessment of language processing 2002, verbal memory 2004, motion perception 2006, shape perception 2008, contrast sensitivity 2010, and spatial attention 2012. The results summary 2000 may determine a quantitative score and pass/fail assessment in relation to functional impairment. More particularly, the sensory-motor neurocognitive assessment associated with the results summary 2000 may result in characterization protocols that may yield response functions relating time and saliency that may generate real-time scores based on: the average final saliency score over three periods, the saliency at which the most time may be spent during testing, and the total time that may be spent in the test.

Additional, the present disclosure may assess the algorithmic fit for an asymptotic function against the response function generated for each sensory-motor neurocognitive assessment protocol. The present disclosure may then assess performance and generate secondary measures, which may include, but are not limited to: 1) basic measures such as the fit parameters, asymptote and area under the curve, 2) comparative measures as the differences between the basic measures of a subject on a particular sensory-motor neurocognitive assessment protocol and that subject from other selected sensory-motor neurocognitive assessment protocols, 3) comparative measures as the differences between the basic measures of a subject on a test and the measures from a selected group of comparison subjects.

Sensory-motor neurocognitive assessment measures associated with the results summary 2000 may be derived in real-time, or near real-time, for each test and may be transformed as standardized scores relative to an age-based comparison group.

These standardized scores may be derived separately for each sensory-motor neurocognitive assessment protocol.

Sensory-motor neurocognitive assessment protocol scores associated with the results summary 2000 may be shown on a radial plot, grouped by cognitive relatedness sensory-motor neurocognitive assessments. Differences between age-normal function and a test subject's function may be colored in particular color to indicate sub-normal function and colored in a different color to indicate supernormal function. Differences that may be induced by the negative impact of invalid cues and the positive impact of valid cues may be shown as closely related functions.

Further, the present disclosure may determine differences between a subject's function and age-normal function from aggregated data.

FIG. 77 is a graphical representation showing functional impairment over time in an exemplary diagnosis summary 2050. Diagnosis summary 2050 may include a clinical diagnosis and/or a recommendation medications listing. The suggested diagnosis summary 2050 may include, but is not limited to, a functional impairment characteristic profile 2052 and one or more suggested diagnoses of specific types of processing impairment and likely or commonly associated underlying pathophysiologies that include conditions, diseases, disorders, intoxications, and other mechanisms of brain functional impairment. Alternatively, specific links to particular pathophysiologies may be established and would be provided in this clinical guide. All such single device diagnostic suggestions are to be considered in the clinical context of testing, and particularly likely or common contextual considerations may also be enumerated and suggested for consideration. In addition, further diagnostic evaluations including further testing on the current device or by other devices or clinical maneuvers may be suggested. These suggestions are intended to clarify the underlying conditions, diseases, disorders, intoxications, and other mechanisms of brain functional impairment may be suggested to assist the individual or involved clinical practitioners in realizing a more complete evaluation 2054. The functional impairment characteristic profile 2052 may be shown graphically on a plot of the rating of the functional impairment characteristic versus the calendar time range 2056. More particularly, the scale for the rating of the functional impairment characteristic of the functional impairment characteristic profile 2052 may range from normal for age 2060 to more impaired 2062.

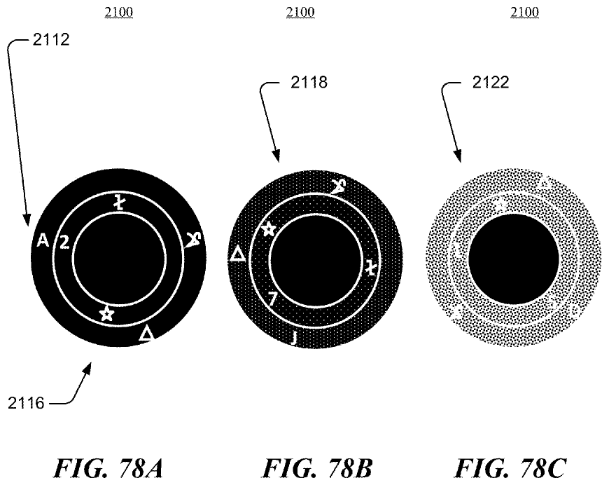

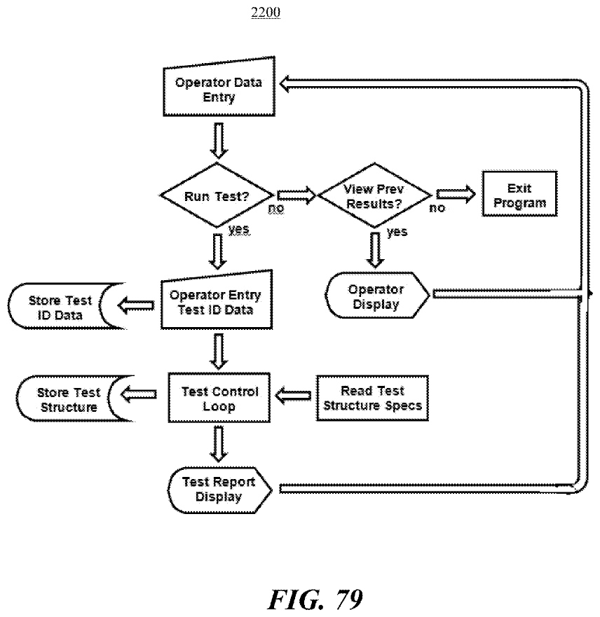

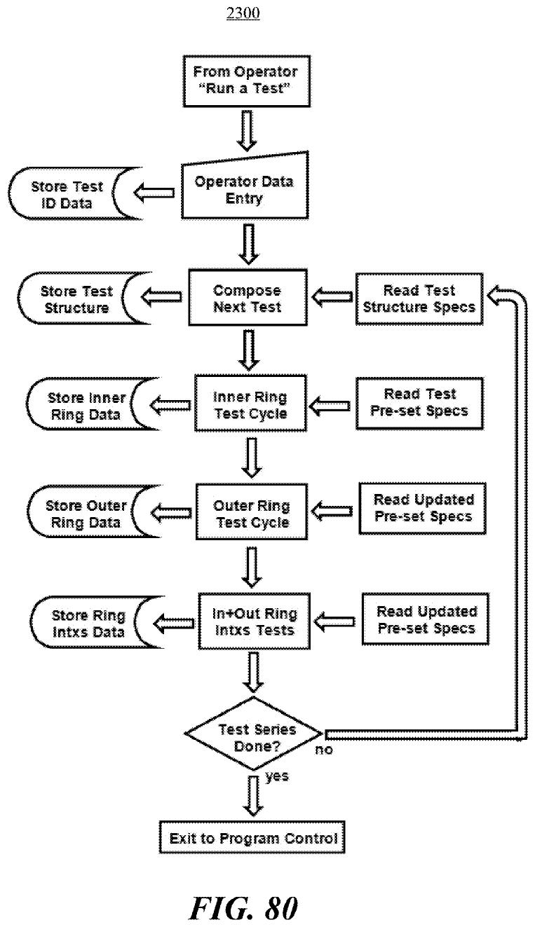

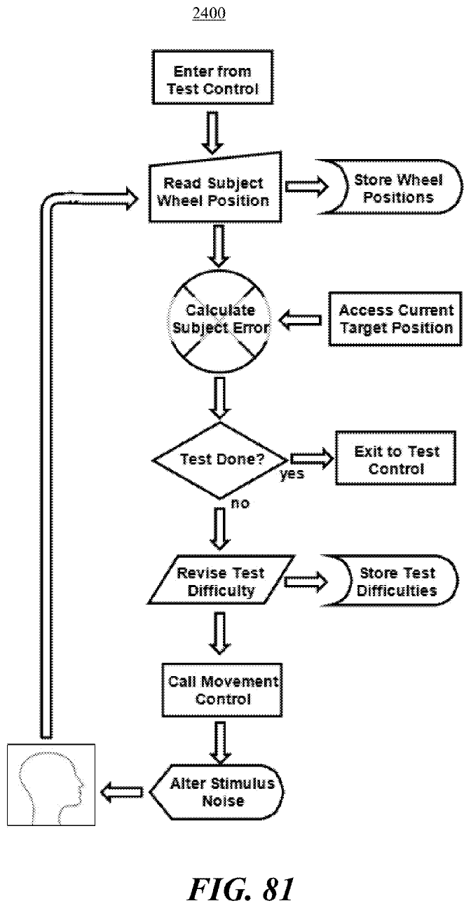

FIG. 78A illustrates aspects of a system 2100 including a display 2112 having two concentric annuli 2116 in a system for automated impairment assessment testing. It will be understood that system 2100 including such a display 2112 including two concentric annuli 2116 may provide initiation of an integration and interaction test 2300 (shown in FIG. 80). Referring to FIG. 80, it will be understood that such an integration and interaction test 2300 may include dual visual stimuli (referenced as “dual stimulus” or “dual stimulus test”). Such a dual stimulus test may include an inner annulus test cycle 2304, outer annulus test cycle 2308 and combined inner annulus and outer annulus test cycle 2312. Such an integration and interaction test 2300 may include evaluating visuo-motor responses by analysis of the sensori-cognito/affecto-motor function in the domains of target movement speed, acceleration, and direction reversal. Referring to FIG. 78A, an exemplary test 2100 may include presenting to the subject (not shown in FIG. 78A) a view of two concentric annular displays, particularly an inner annular display and an outer annular display, which may be separated by a thin annular gap (schematized in FIG. 78A). Test 2100 facilitates the receiving and recording of inputs tracking data, such as response wheel position data (such as reading and storing response wheel positions 2412, as shown in FIG. 81). The subject manually rotating such a response wheel (not shown) provides such inputs as otherwise described hereinabove as the subject attempts to control the inner annular display (see FIG. 78A), where the inner annular display may correspondingly rotate about a center of the two concentric annular displays. In some embodiments, correlation between the rotation of the input and the rotation of the inner annular display may be systematically deviated during test administration as gain, speed, or offset changes. In addition to the test input linking an inner annulus to a control device, the environment may include an outer annular display (see FIG. 78A) configured to rotate in accordance with an algorithm for controlling variables such as, for example, display characteristics, subject performance, and other variables. In some embodiments, the test may be configured to receive inputs from a subject attempting to align the target in the inner annulus with that in the outer annulus and then to maintain that alignment throughout a period of outer annulus algorithmic display changes that rotate the location of the target. The inner and out annuli may contain a number of imbedded targets and foils (see FIG. 78A). As used herein, “targets” are stimuli having some specific characteristics that distinguish them as the goal item in an ongoing stimulus-response task. As used herein, “foils” are stimuli that are of that same class as the target but do not share the complete set of defining characteristics of the target. In addition to target location, the device may alter which targets and foils are presented at a given time, the target's and foil's perceptual salience, and the subject's response characteristics as registered through a subject interface response device and is recorded by the system.

In embodiments, a system may include one or more foils. It will be understood that in methods as disclosed, one or more foils may be displayed. In an embodiment, one or more foils may be provided to the display, for example, from functioning of a Foils Parameters module.

Referring to FIGS. 78A, 78B and 78C, in embodiments the two annular stimulus areas may each display one or more selected stimulus types from a group of identified test domains (e.g., letters, numbers, words, symbols, shapes, faces, motion, optic flow, kinetic edges, etc.). A test domain selected for display in one annulus may be the same as, or different from, a test domain to be displayed in the other annulus. A test domain selected for display in one annulus, or the other annulus, may be the same as, or different from, another test domain selected for display in the one annulus, or in the other annulus, in a previous display in a series presented to the subject or in a previous or subsequent test of the subject. A single target from the selected domain may be presented in the assigned annulus (e.g., one letter for the outer annulus and one number for the inner annulus). Multiple foil stimuli (non-targets of the same or a different type) may be distributed around the non-target areas of the annuli. The foil types may be the same or different within or between the two annuli or the same or different from those in previous or subsequent tests. The location of foils and targets, as well as display and response characteristics, may be continuously varied (direction and speed of movement), varied in a discrete manner, or varied in any suitable manner during testing, such as, for example, by being varied continuously based on pre-set test condition parameters and subject performance in the same test, current test, the same test performed previously, and/or other tests.

A person of ordinary skill in the art will understand that specific applications of the present disclosure will include many variations. For the purposes of clarity, exemplary embodiments of systems and methods for testing described herein. It should be understood that application of the present disclosure may utilize some or all of the steps in exemplary embodiments. Furthermore, additional steps may be included. The steps may be performed in order, or alternatively in different orders. The term “sequence” as used herein refers to presentation of a specific item or specific items in a continuous or discontinuous sequence from an otherwise defined set of stimuli, e.g., vowels from the set of letters, or spoons from the set of eating utensils. The term “set” as used herein refers to a complete list of specific items that may be used in testing in the domain of those items. “Domain” refers to a superset or supersets from which a set may be drawn, e.g., the set of letters from the domain of all symbols and from the domain of all shapes; or e.g., the set of eating utensils from the domain of manual tools or from the domain of household items.

Referring to FIG. 80, in embodiments a system 2300 for performing an integration and interaction test may include implementation of Sequence A by an inner ring test cycle module 2304. Sequence A implementation may provide as follows: The inner annulus may contain a single target cursor element shown at 100% signal-to-noise ratio (SNR) that may be moved by subject wheel rotation. The outer annulus targets and foils may be moved separately, for example, by the inner ring test cycle module 2304 or another suitable module. The system 2300 may be configured to receive inputs from the subject responding to changes in the position, direction, and speed of the outer target through changes in the outer target SNR, initially, with no foils, then with 1, 2, and/or 3 non-target foils of another type (e.g., shapes instead of letters). In an embodiment, the amount of non-target foils may change within one run of the Sequence A of the integration and interaction test. In an embodiment, the amount of non-target foils may not change within one run of the Sequence A of the integration and interaction test.

Referring to FIG. 80, in embodiments, an integration and interaction test may include implementation of Sequence B by an outer ring test cycle module 2308. Sequence B implementation may provide as follows: The outer target with a single element at 100% SNR may be moved. The inner target cursor may change SNR initially, with no foils, then with 1, 2, and then 3 non-target foils of other class (e.g., shapes instead of letters). While the outer target is moved by the outer ring test cycle module 2308, the subject may try to match the position, direction, and speed of the outer target across inner SNR and foils changes. In embodiments, the amount of non-target foils may change within one run of the Sequence B of the integration and interaction test. In embodiments, the amount of non-target foils may not change within one run of the Sequence B of the integration and interaction test.

Referring to FIG. 80, in embodiments, a system 2300 providing an integration and interaction test may include implementation of Sequence C by a combination test cycle module 2312. Sequence C implementation may provide as follows: The inner target may start at a critical SNR and target speed derived for the subject in Sequence B and may be moved by the subject. The outer target may start at critical SNR and target speed derived for the subject in Sequence A and may be moved by the combination test cycle module 2312. The subject may try to match the position, direction, and speed of the outer target through successive changes in the inner and outer SNRs, initially, with no foils, then with 1, 2, and then 3 foils of other (non-target) classes (e.g., shapes instead of letters) successively and separately added to the inner and outer annuli. In embodiments, the amount of non-target foils may change within one run of the Sequence C of the integration and interaction test. In embodiments, the amount of non-target foils may not change within one run of the Sequence C of the integration and interaction test.

Referring to FIG. 80, in embodiments, an integration and interaction test may include implementation of Sequence D by the combination test cycle module 2312. Sequence D implementation may provide as follows: The inner target may start at Sequence B critical SNR and target speed and may be moved by the subject. The outer target may start at Sequence A critical SNR and target speed and may be moved by the combination test cycle module 2312. Before each test run, a pair of stimuli may be presented in the center consisting of one element from the outer annulus class and one element from the inner annulus element class. In embodiments, the two specific elements must be aligned for successful performance, ignoring the other elements of the same classes in their respective annuli. Initially, there may be just one element of each class in the respective annuli. Subsequently, there may be non-target foils of the target class added to the respective annuli, first 1, 2, and then 3 non-target foils of the target class separately added to each annulus. The subject may try to match the position, direction, and speed of the outer target through separate changes (SNRs and number of foils) in both the inner and the outer annuli. In embodiments, the amount of non-target foils may change within one run of the Sequence D of the integration and interaction test. In embodiments, the amount of non-target foils may not change within one run of the Sequence D of the integration and interaction test.

Referring to FIG. 80, in embodiments, an integration and interaction test may include implementation of Sequence E by a combination test module. Sequence E implementation may provide as follows: The inner target may start at Sequence B critical SNR and target speed and may be moved by the subject with one of two response wheels. The outer target may start at the Sequence A critical SNR and target speed and may be moved by the subject with the other of two response wheels. Both annuli may move at different speeds and directions under program control and the subject may move the two wheels to keep the targets in the concentric circles aligned. Initially, there may be just one element of each class in the respective annuli. Subsequently, there may be non-target foils of a non-target class added to the respective annuli, first with 1, 2, and then 3 non-target foils of the non-target class added separately to the two annuli. The subject may try to match the position, direction, and speed of the inner and outer targets through changes in both the inner and the outer annuli (SNRs and number of foils). In embodiments, the amount of non-target foils may change within one run of the Sequence E of the integration and interaction test. In other embodiments, the amount of non-target foils may not change within one run of the Sequence E of the integration and interaction test.

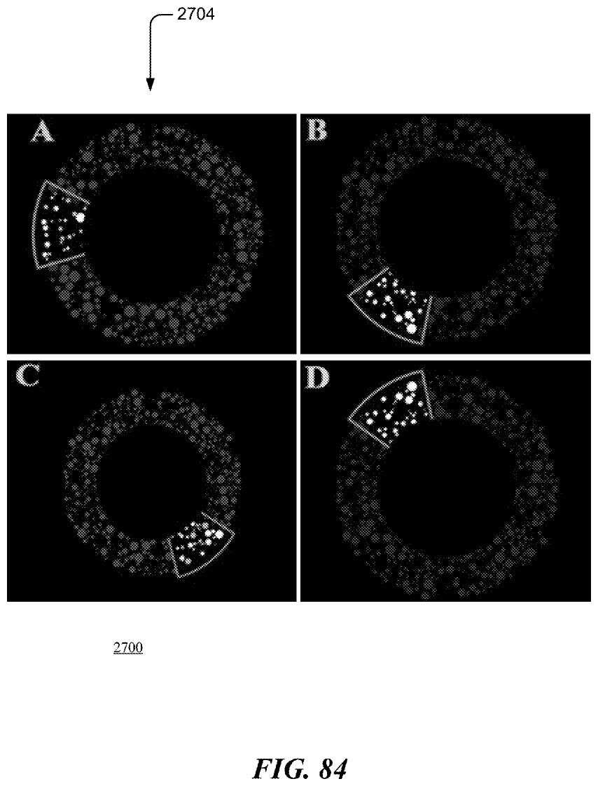

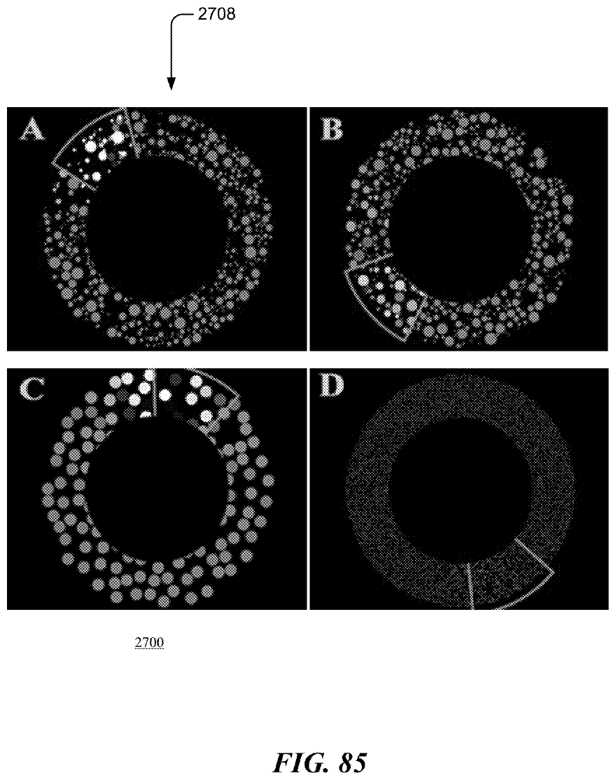

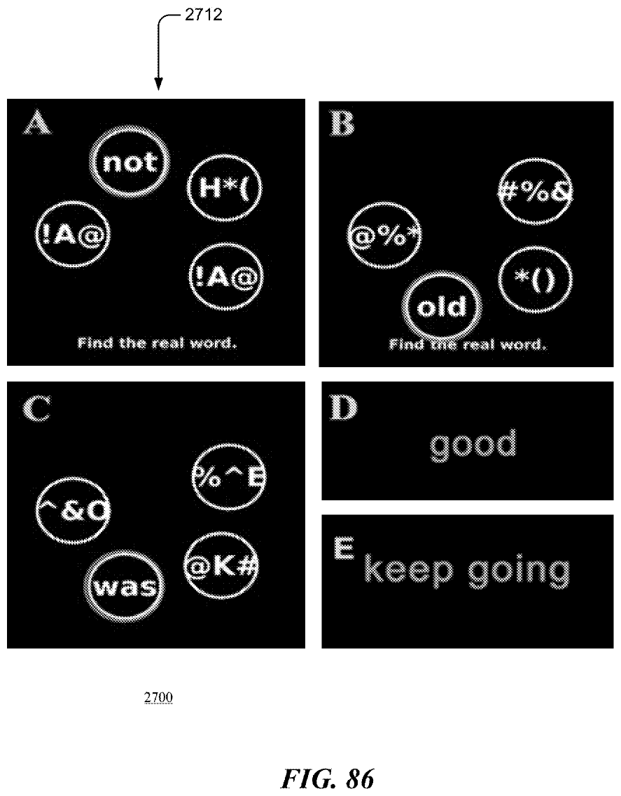

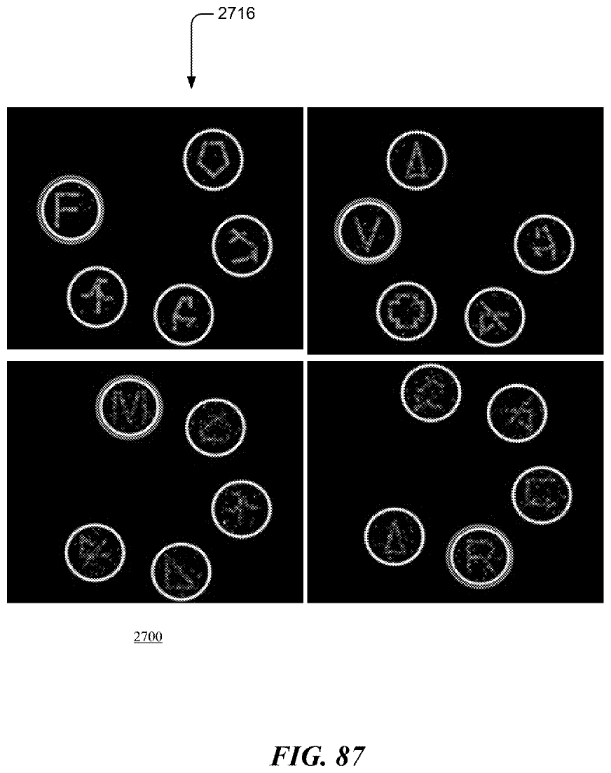

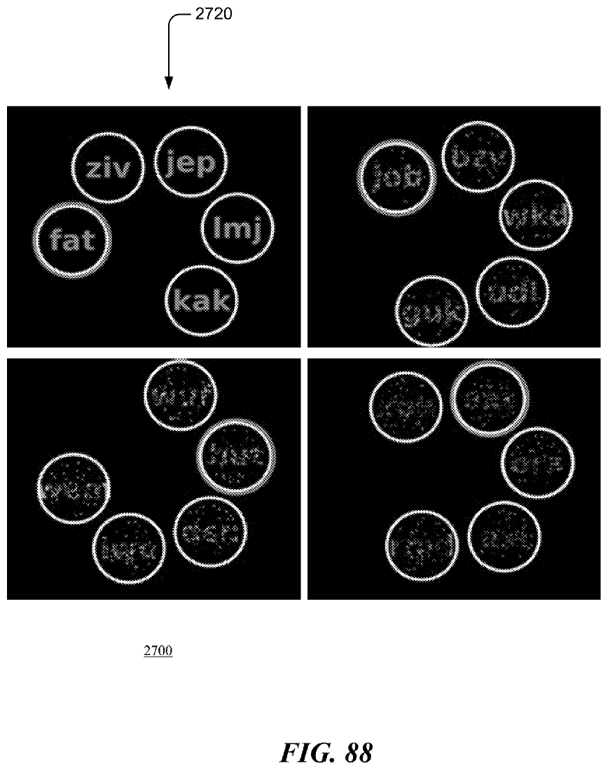

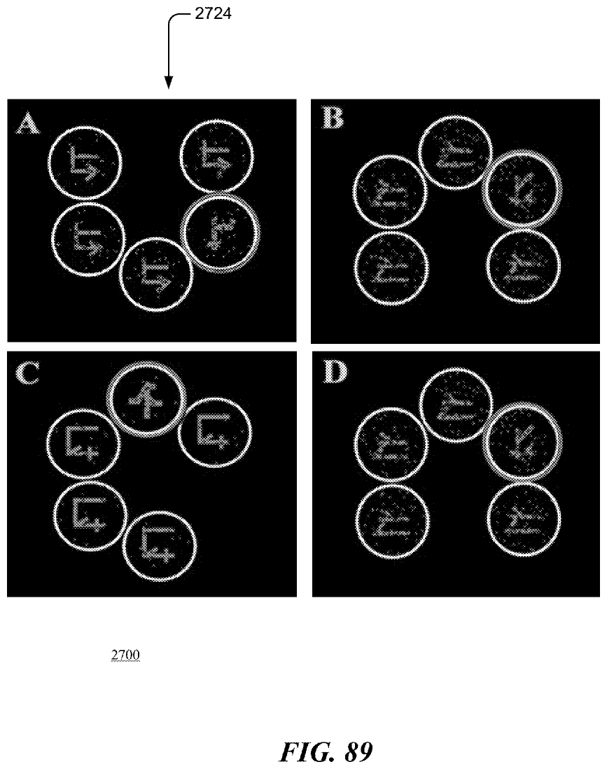

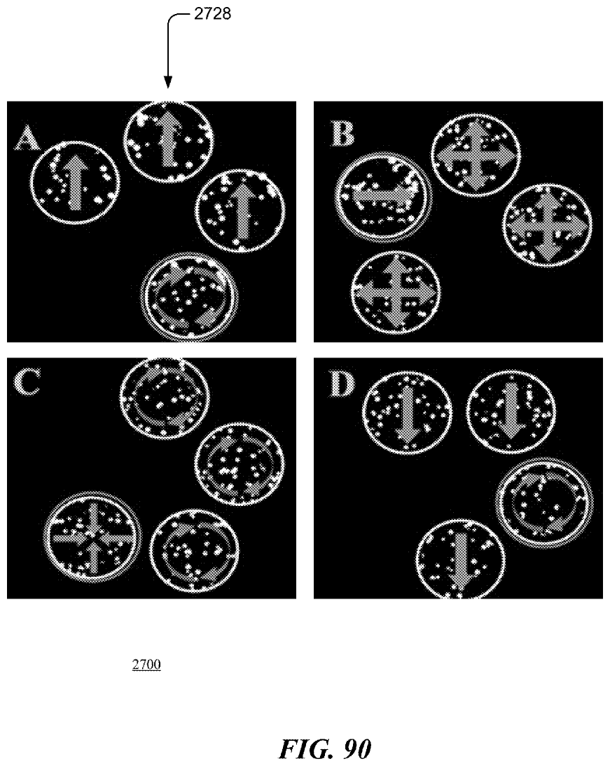

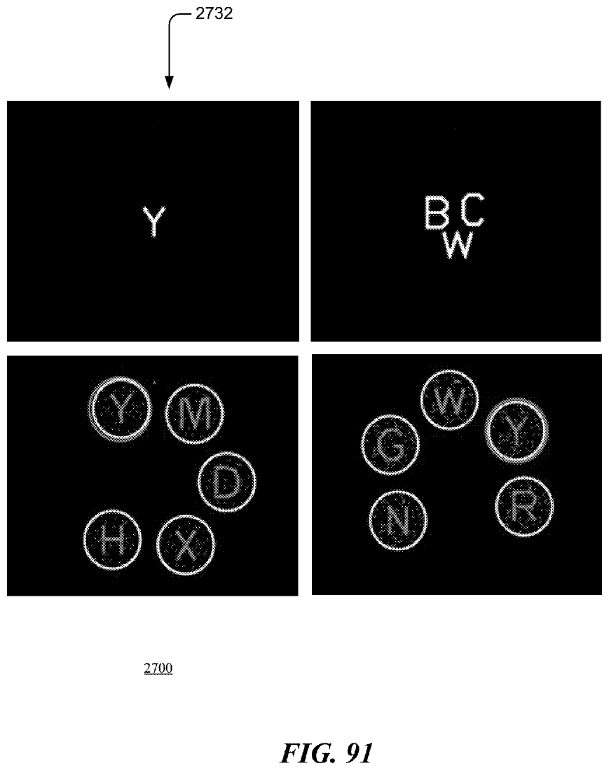

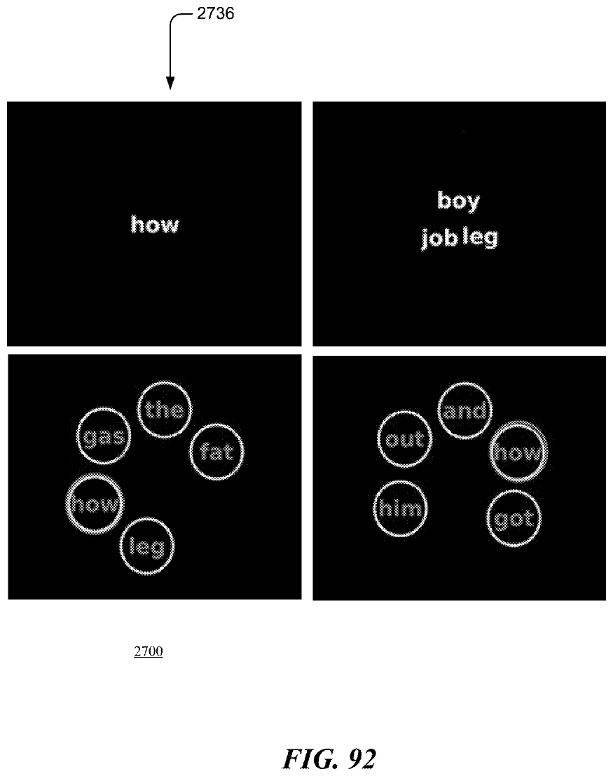

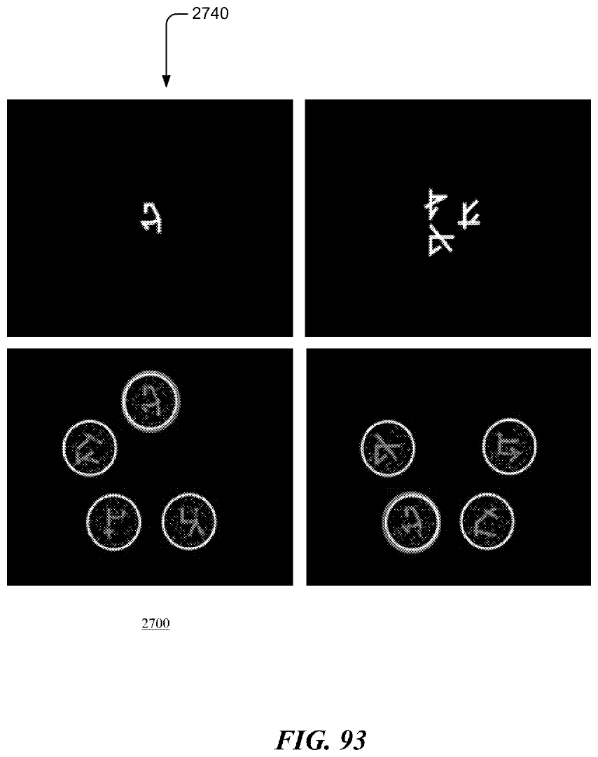

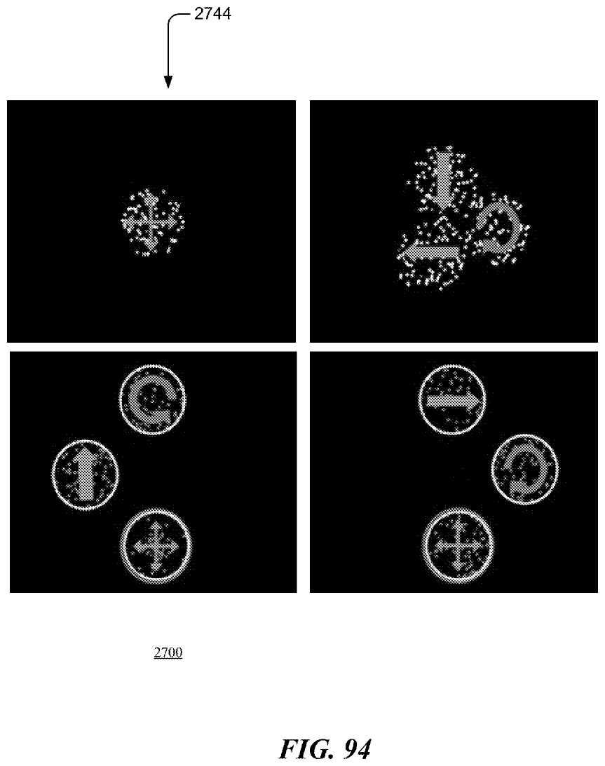

Referring to FIGS. 84-94 in embodiments an integration and interaction test 2700 may include Set A of Letters provided by a Letters module. Set A (Letters) may include English (or other) language letters with imposed atypicality that may be presented as a continuous variable (i.e. salience) affecting the detection and discrimination of elements in this set. Variables that may be controlled and changed in such a test include: Clutter, Orientation, Brightness, Size, and Thickness. As used herein, “Clutter” means pixel degeneration of the letter, character, etc. with pixel addition outside of the confines of the character. As used herein, “Orientation” means rotation around the center-of-mass of the character in, or out of, the plane of the screen. As used herein, “Brightness” means dimming character lines/pixels with linked or separate changes in background luminance. As used herein, “Size” means dimensional size of characters. As used herein, “Thickness” means lines, dots, or make up characters, which may be made larger or smaller.

Referring to FIG. 84-94 in embodiments an integration and interaction test may include, for example, a Set B of Words provided by a Words parameter module. Such Set B (Words) may include English (or other) language words with imposed atypicality presented as a continuous variable (e.g. SNR) affecting the detection and discrimination of elements in this class.

Referring to FIG. 84-94, in embodiments an integration and interaction test may include, for example, Set C of Regular Shapes characteristics provided by a Regular Shapes parameters module. Such Set C (Regular Shapes) may include geometric shapes (e.g., circles, squares, triangle, etc.) with imposed atypicality presented as a continuous variable (e.g. SNR) affecting the detection and discrimination of elements in this class.

Referring to FIGS. 84-94, in embodiments an integration and interaction test may include, for example, a Set D of Irregular Shapes characteristics that may be provided by an Irregular Shapes parameters module. Such a Set D (Irregular Shapes) may include irregular line and curve sets with branching and intersecting composition forming open and closed areas (e.g., false fonts) with imposed atypicality presented as a continuous variable (e.g. SNR) affecting the detection and discrimination of elements in this class.

Referring to FIGS. 84-94, in embodiments an integration and interaction test may include, for example, a Set E of Planar Motion characteristics that may be provided by a Planar Motion parameters module. Such a Set E (Planar Motion) characteristics may include, for example, uniform movement of dots, lines, or dot/line patterns with imposed parametric changes presented as a continuous variable (e.g. SNR) affecting the detection and discrimination of the planar motion direction, speed, or acceleration.

Referring to FIGS. 84-94 in an embodiment, an integration and interaction test may include, for example, a Set F of Optic Flow characteristics that may be provided by an Optic Flow parameters module. Such Set F (Optic Flow) characteristics may include, for example, uniform movement of dots, blobs, or shapes moving in radial, circular, or shear patterns (or combinations of those patterns) simulating the visual scene observed during self-motion through the environment with imposed parametric changes presented as a continuous variable (e.g. SNR) affecting the detection and discrimination of elements in this class.

Referring to FIGS. 84-94 in an embodiment an integration and interaction test may include, for example, a Set G of Kinetic Edges that may be provided by a Kinetic Edges parameters module. Such Set G (Kinetic Edges) may include regionally distinct coherent movement of dots, blobs, or shapes moving in two or more adjacent or approximate area to form an edge between those areas that is visible because of the perception of the differences in the motion with imposed parametric changes presented as a continuous variable (e.g. SNR) affecting the detection and discrimination of elements in this class.

Referring to FIGS. 84-94 in an embodiment an integration and interaction test may include, for example, a Set H of Motioned Defined Objects that may be provided by a Motioned Defined Objects parameters module. Such Set H (Motioned Defined Objects) may include parameters of, for example, spatially linked coordinated movement of dots, blobs, or shapes moving to simulate that of an animate or inanimate object with imposed parametric changes presented as a continuous variable (e.g. SNR) affecting the detection and discrimination of elements in this class.

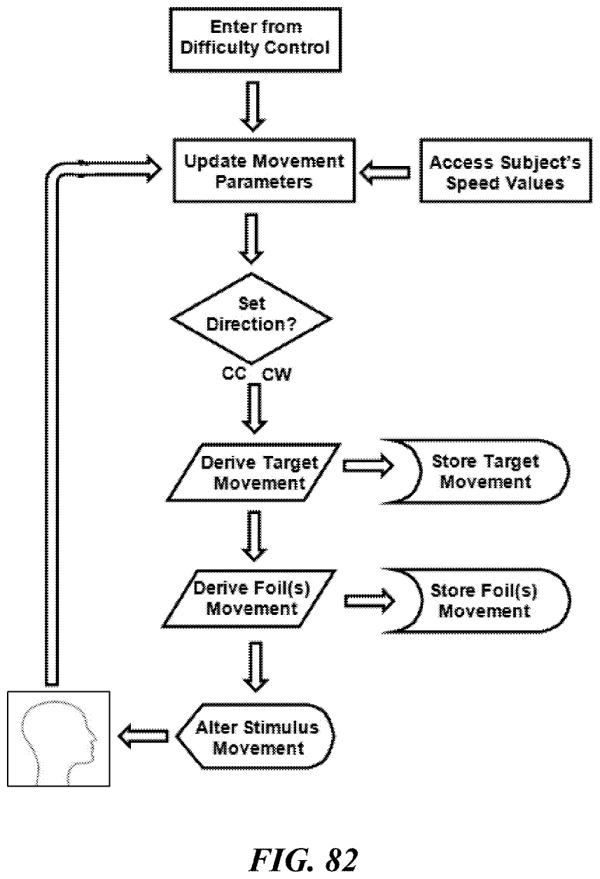

Referring to FIG. 82, systems and methods according to disclosed subject matter may include imposing parametric changes in task difficulty (SNR) and scoring input results along parameters of speed, accuracy, and adaptability in relation to such parametric changes in task difficulty (SNR). In embodiments, for example, an imposed parameter may include length or duration of the testing period. In embodiments, for example, an imposed parameter may include linking length or duration of the testing period to relationships between stimulus and response characteristics.

Referring to FIG. 82 in embodiments, for example, a parameter of sequential changes in test domains may be included. Such sequential changes in test domains may, for example, create series of sub-tests that may be scored separately, with summary scoring derived from the administered series of sub-tests. Referring to FIG. 82, in embodiments including a sequential changes parameter such as, for example, sequence A and B, each yields data that may be represented in an SNR/Speed/Accuracy 3-space. Inflection points may be used as starting values, or critical starting values, for other sequences that manipulate SNR from that critical level.

Referring to FIGS. 78A, 78B and 78C embodiments of the present disclosure may include a system configured for invoking responses in two restricted sets of cortical areas. Such an embodiment may include, for example, two concentric annuli as shown generally in FIGS. 78A, 78B and 78C. Through application of a module, two concentric annuli as shown generally in FIGS. 78A, 78B and 78C may be displayed, for example, to invoke responses in two restricted sets of cortical areas. Referring to FIG. 81, in some embodiments a system 2400 may include a stimulus control module 2404 having at least one stimulus noise modulation parameter for controlling and altering stimulus noise. Referring to FIG. 82, in an embodiment system 2500 may include foils control module 2508 having to include at least one foils control parameter for focusing on at least one specific target-foil domain. Referring to FIG. 81, in an embodiment system 2400 may include task characteristics control module 2408 having at least one task characteristics or task difficulty parameter. In embodiments, an inputs recording module 2412 may record tracking inputs in response to the stimuli, foils and tasks, for testing or assessment of processing characteristics of each cortical-subcortical network during their combined and interacting activation.

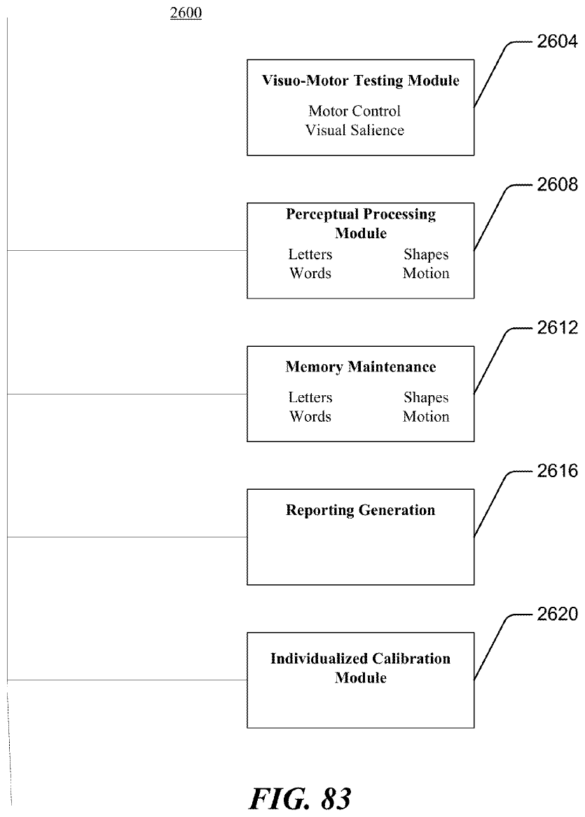

Referring to FIG. 83, a system 2600 may include: visuo-motor testing module 2604, perceptual processing module 2608, memory maintenance module 2612, reporting generation module 2616 configured to generate a combined activation stimulus-response profile that reflects interactions between the activated networks of the brain, and individualized calibration module 2620. System 2600 may also include a module configured to probe such interactions with respect to, for example, continuous cost/benefits of co-activation, and also the discontinuous cost/benefits of intermittent co-activation, which may be triggered or produced by stimulus-task modulation or by intrinsic processes consequent to co-activation of networks.

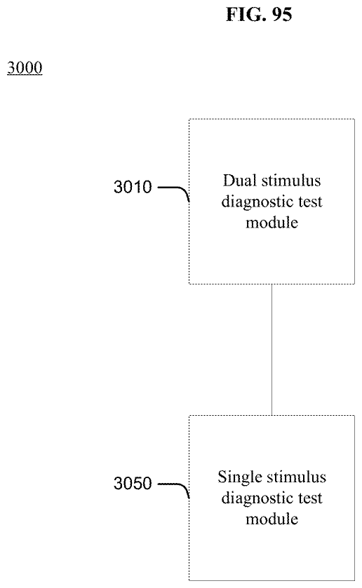

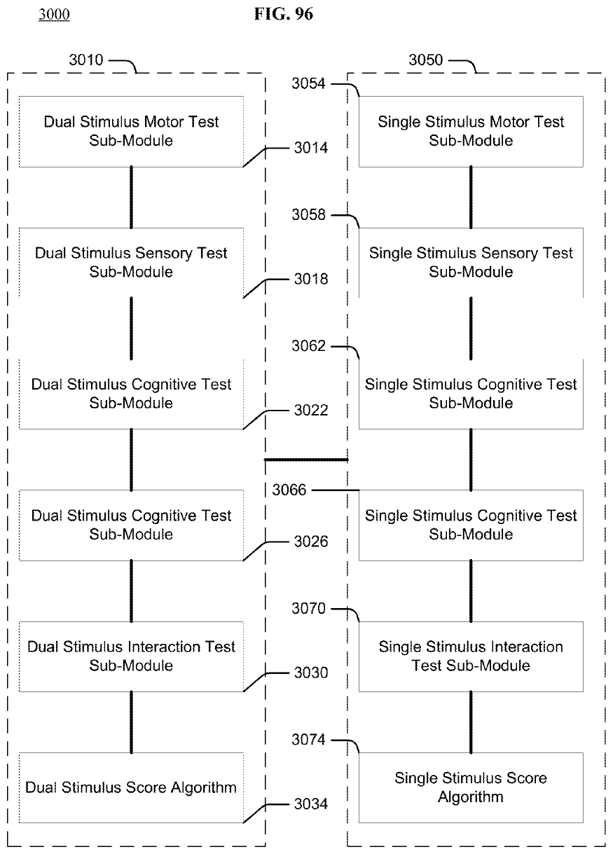

Referring to FIG. 95, a system 3000 may include both a dual stimulus testing module 3010 and a single stimulus testing module 3050. As shown in FIG. 96, dual stimulus testing module 3010 may include a dual stimulus motor test sub-module 3014. Dual stimulus testing module 3010 may include a dual stimulus sensory test sub-module 3018. Dual stimulus testing module 3010 may include a dual stimulus cognitive test sub-module 3022. In some embodiments, dual stimulus testing module 3010 may include a second dual stimulus cognitive test sub-module 3026. Dual stimulus testing module 3010 may include a dual stimulus interaction test sub-module 3030. Dual stimulus testing module 3010 may include a dual stimulus score algorithm 3034. Single stimulus testing module 3050 may include a single stimulus motor test sub-module 3054. Single stimulus testing module 3050 may include a single stimulus sensory test sub-module 3058. Single stimulus testing module 3050 may include a single stimulus cognitive test sub-module 3062. In some embodiments, single stimulus testing module 3050 may include a second single stimulus cognitive test sub-module 3066. Single stimulus testing module 3050 may include a single stimulus interaction test sub-module 3070. Single stimulus testing module 3050 may include single stimulus score algorithm 3074.

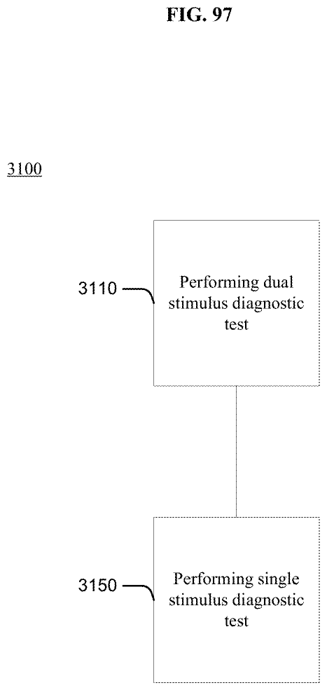

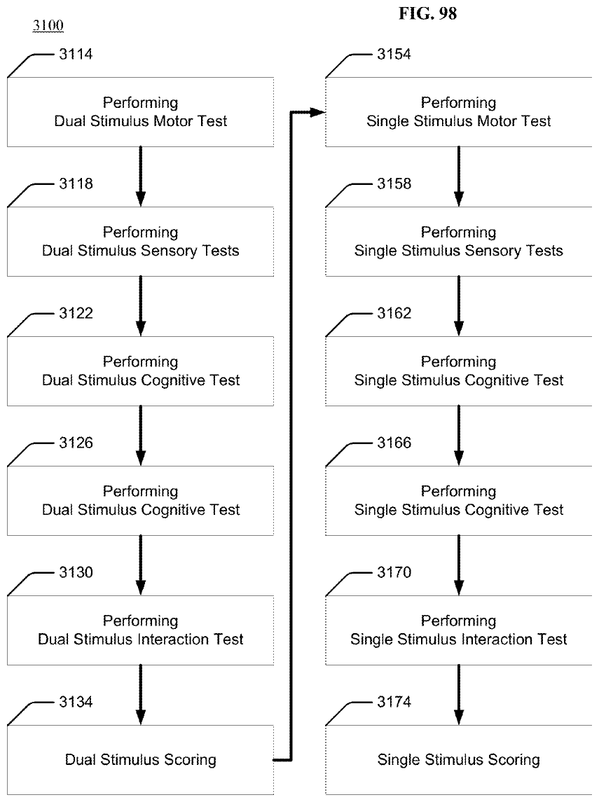

Referring to FIG. 97, a method 3100 for automated visual impairment testing may include both performing 3110 dual stimulus tests and performing 3150 single stimulus tests. As shown in FIG. 96, performing 3110 dual stimulus tests may include performing 3014 dual stimulus motor tests. Performing 3110 dual stimulus tests may include performing 3118 dual stimulus sensory tests. Performing 3110 dual stimulus tests may include performing 3122 dual stimulus cognitive tests. In some embodiments, performing 3110 dual stimulus tests may include performing 3126 second dual stimulus cognitive tests. Performing 3110 dual stimulus tests may include performing 3130 dual stimulus interaction tests. Performing 3110 dual stimulus tests may include dual stimulus scoring 3134. Performing 3150 single stimulus tests may include performing 3154 single stimulus motor tests. Performing 3150 single stimulus tests may include performing 3158 single stimulus sensory tests. Performing 3150 single stimulus tests may include performing 3162 single stimulus cognitive tests. In some embodiments, performing 3150 single stimulus tests may include performing 3166 second single stimulus cognitive tests. Performing 3150 single stimulus tests may include performing 3170 single stimulus interaction tests. sub-module 3070. Performing 3150 single stimulus tests may include single stimulus scoring 3074.

Disclosed subject matter may provide automated quantitative assessment of functional impairment in individuals by performing automated visual motor response testing, and may provide reports of the same. Such automated quantitative assessment of functional impairment in an individual may comprise automated dual stimulus testing. In some embodiments, dual stimulus testing may be performed alone to provide quantitative assessment of functional impairment. In embodiments, dual stimulus testing may be performed in combination with single stimulus testing. For example, dual stimulus testing may be performed for initial assessment or diagnosis, in combination with single stimulus testing which may be performed for detailed assessment or diagnosis. According to the disclosure, systems and methods may perform automated functional impairment testing that quantitatively measures and assesses response characteristics of the brain in a subject.

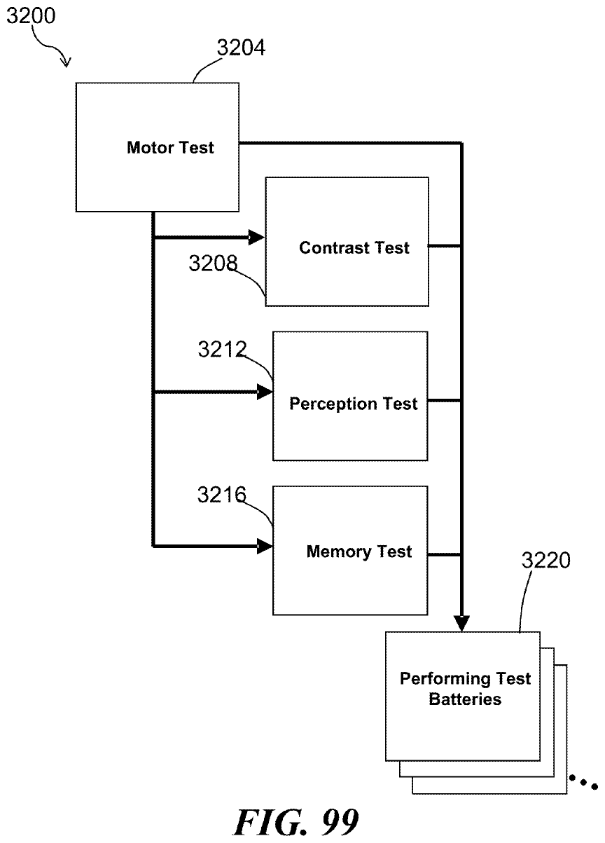

FIG. 99 shows a paradigm of a hierarchical nature of parametric individualization. In the exemplary hierarchy, 3200, the resulting data from a movement test 3204 may be applied to a visual saliency test 3208, a perception test 3212, and/or a memory test 3216. Each of these tests may be stored in a database, and each test may be performed 3220 by operation of the specific module.

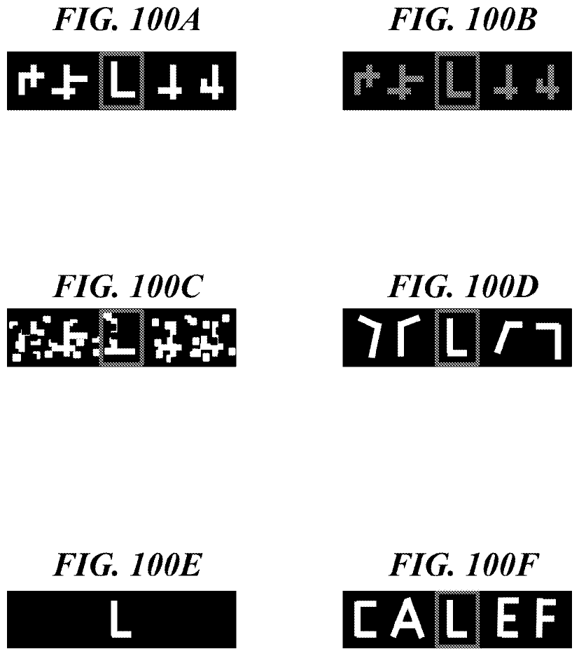

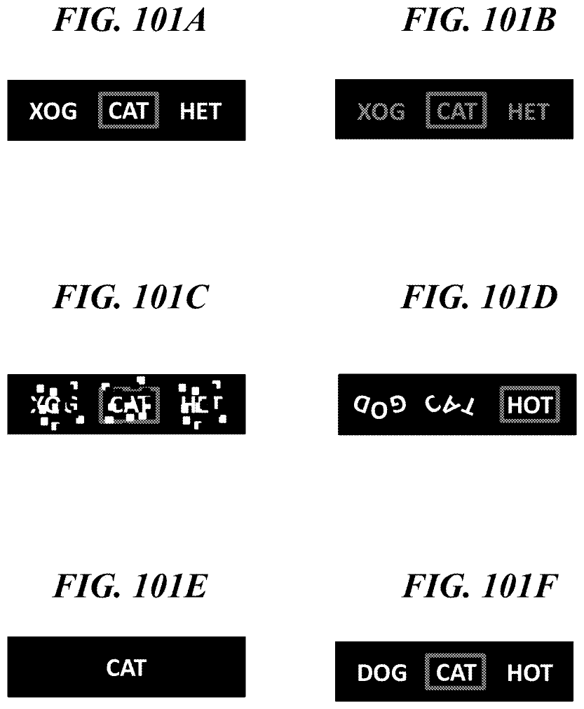

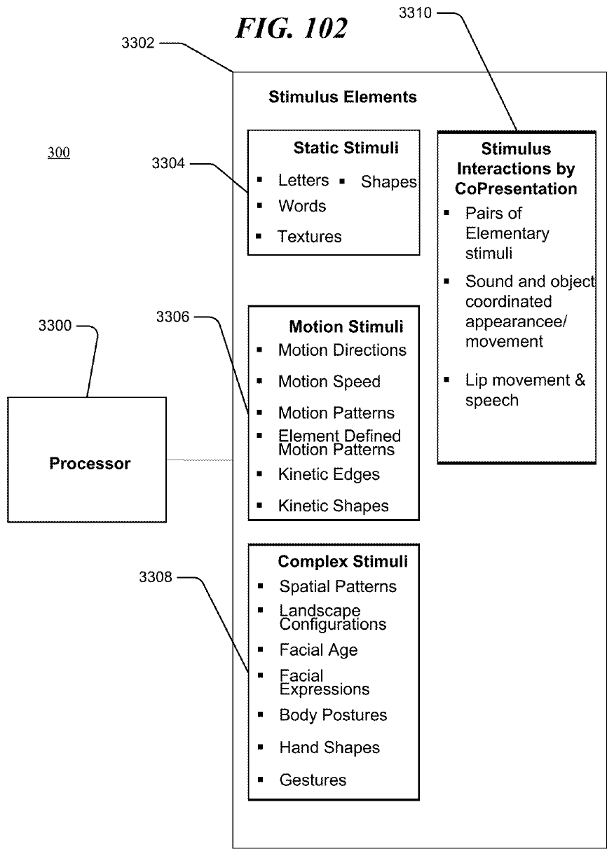

FIGS. 100A, 100B, 100C, 100D, 100E, and 100F as well as FIGS. 101A, 101B, 101C, 101D, 101E, and 101F detail exemplary visual depictions of a series of test scenes that may be employed by exemplary embodiments. Embodiments may employ a variety of stimuli throughout the testing environments. Exemplary stimuli that may be employed are shown in FIG. 102. As shown, exemplary embodiments may employ static stimuli 3304, including but not limited to letters, shapes, words, and textures. The exemplary embodiments may also employ motion stimuli 3306, including but not limited to, motion directions, motion speed, motion patterns, element defined motion patterns, kinetic edges, and kinetic shapes. Exemplary embodiments may also include complex stimuli 3308, including but not limited to, spatial patterns, landscape configurations, facial age, facial expressions, body postures, hand shapes, and gestures. Exemplary embodiments may further include stimulus interactions by co-presentation 3310, including but not limited to, pairs of elementary stimuli, sound and object coordinated appearance or movement, and lip movement and speech. In some embodiments, these stimuli may be used independently or in combination. Use of the stimuli may be executed by the computer processor in accordance with system rules.

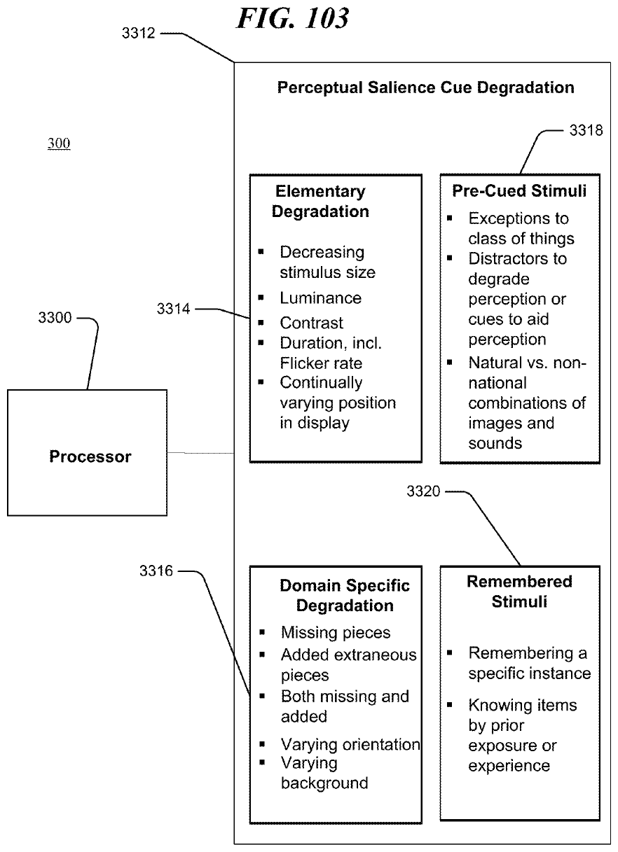

An exemplary rule, or heuristic model, employed by embodiments of the present disclosure includes perceptual salience cue degradation, as shown in FIG. 103. As shown, exemplary tests, including memory and perception tests, may employ a variety of means to distort the ease of recognition of the stimuli. An exemplary means for distorting recognition of the stimuli, includes elementary degradation 3314. Elementary degradation 3314 may include, but is not limited to, decreasing stimulus size, luminance, contrast, duration and flicker rates, and continually varying stimulus position. Embodiments may also employ domain specific degradation 3316. Domain specific degradation 3316 may include, but is not limited to, missing pieces of stimuli, adding extraneous pieces to stimuli, a combination of missing and extraneous pieces, varying orientation, and varying background. Embodiments may also employ pre-cued stimuli 3318. Pre-cued stimuli may include, but are not limited to, class exceptions, perception distractors, perception aids, natural combinations of images and sounds, and non-natural combinations of images and sounds. Embodiments may also employ remembered stimuli 3320. Exemplary remembered 3320 stimuli may include, but are not limited to, specific instance remembering, and knowledge of prior items or prior exposure.

Further examples of heuristics that may be employed include the use of Words vs. non-word letter sets, decreasing the contrast of the target, adding static noise elements (e.g., obscuring dots), adding positional noise (e.g., shaking), and adding geometric distortions (e.g., twisted), and presentation changes (e.g., letters in words closer together).

Furthermore, memory and perception tests modules may be configured for determining accuracy of subject inputs in identified matching/not-matching words across different modality (e.g., spoken words in the auditory modality), specific word identified by pre-cueing a specific location in the display (e.g., a flashed red dot preceding a list of words, the word at the flashed location being the target) where the target will be subsequently presented, specific words cued conceptually, either specifically (e.g., a common word) or categorically (e.g., your job or name), etc.

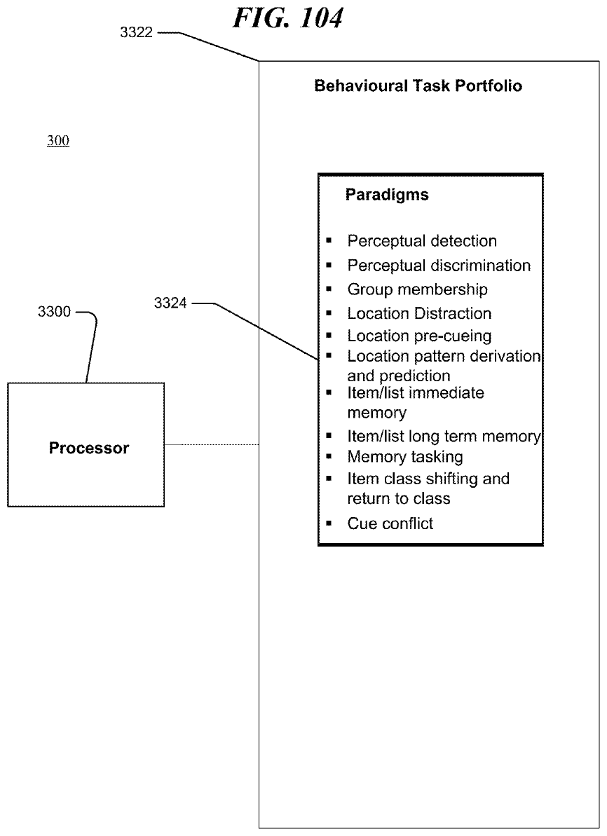

FIG. 104 is a simplified block diagram illustrating exemplary behavioral tasks 3322 that may be employed by embodiments of the present disclosure. As shown, exemplary paradigms 3324 that may be used include but are not limited to, perceptual detection, perceptual discrimination, group membership, location distraction, location pre-cueing, location pattern derivation and prediction, item/list immediate memory, item/list long-term memory, memory masking, item class shifting and return to class, cue conflict.

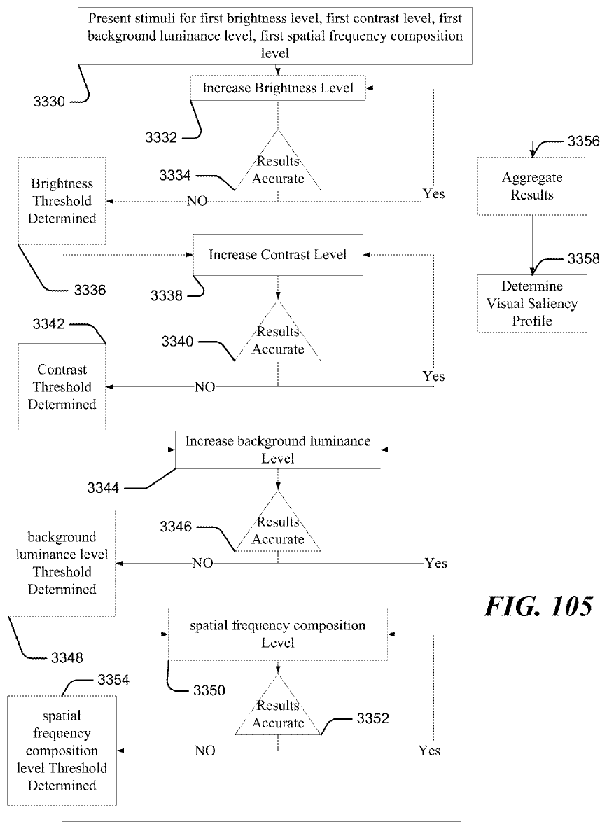

FIG. 105 illustrates an exemplary heuristic model that may be employed by embodiments of the present disclosure. As shown, the system may determine a visual saliency profile 3358 by initially presenting a stimuli for a default 3330, or pre-set brightness, contrast, background luminance, and spatial frequency composition. In accordance with rules of the system, the system may vary each of brightness 3332, contrast 3338, background luminance 3344, and spatial frequency composition 3350 in a chosen order, individually, or in combination, to determine a threshold value at which each parameter inputs become inaccurate. Upon determination of each threshold value, a system may employ an aggregation algorithm 3356 to determine a visual saliency profile. In some embodiments, this visual saliency profile may be deployed or considered in the determination or deployment of subsequent tests.

The invention in one aspect is a method of detecting preclinical Alzheimer's Disease (“AD”), which combines the following screening tests: adaptive motor response; visual salience; shape discrimination and visual motor reaction time. The combined output of the four screening tests may be used to detect preclinical AD. In another aspect, a computing system is configured to perform the method. As used herein, preclinical AD is defined as normal cognitive performance but with abnormal AD biomarkers (exhibiting one or more of reduced AB42/40 ratio, increased amyloid PET SUVR, elevated pTau217 and pTau181).

As used herein, the acronym “MCI” refers to what is known as Mild Cognitive Impairment. MCI is a condition in which people have more memory or thinking problems than other people their age. The symptoms of MCI are not as severe as those of AD or a related dementia. People with MCI can usually take care of themselves and carry out their normal daily activities.

As used herein, the acronym “RAVLT” refers to the Ray Auditory Verbal Learning Test. As one skilled in the art will know, RAVLT evaluates a wide diversity of functions: short-term auditory-verbal memory, rate of learning, learning strategies, retroactive, and proactive interference, presence of confabulation of confusion in memory processes, retention of information, and differences between learning and retrieval. Participants are given a list of 15 unrelated words repeated over five different trials and are asked to repeat. Another list of 15 unrelated words are given and the client must again repeat the original list of 15 words and then again after 30 minutes. Approximately 10 to 15 minutes is required for the procedure (not including 30 min. interval).

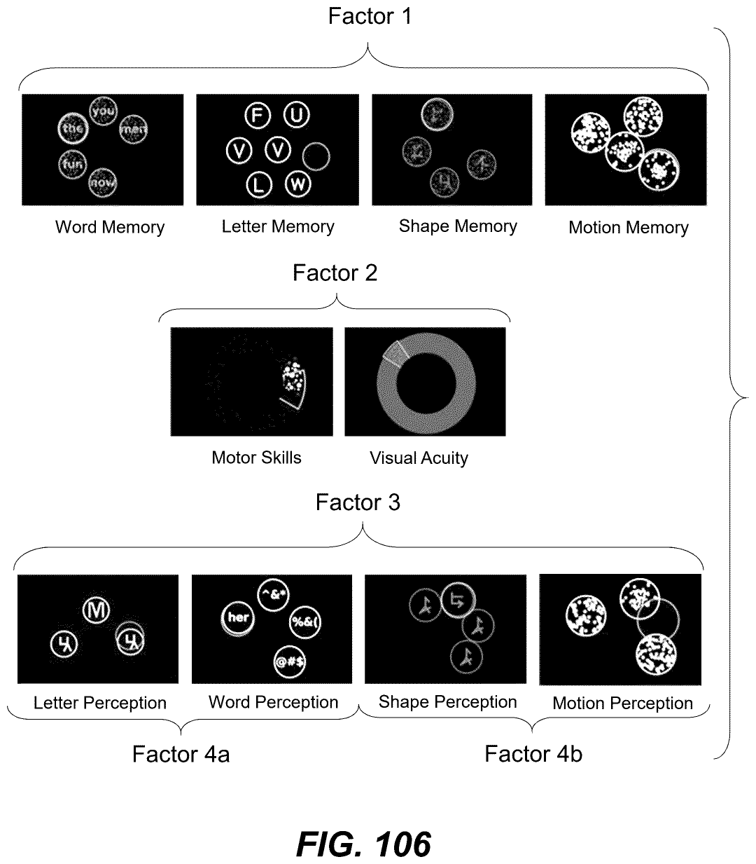

To evaluate and validate the effectiveness of the Cognivue Test (see FIG. 106, and discussion thereof, below), Applicant initiated a study (the “Bio-Hermes study”) having a sample comprising 1003 individuals, resulting in 960 valid, complete records for statistical analysis. Unadjusted analyses were performed with these 960 records as discussed in greater detail below. During the Bio-Hermes study, a Cognivue CLARITY device was used to conduct the Cognivue Test which evaluates six cognitive domains, as well as two performance parameters.

As shown in FIG. 106, the Cognivue Test comprises four memory tasks-Word, Letter, Shape, and Motion (the “Memory Tasks”); four discrimination tasks-Letter, Word, Shape, and Motion (the “Discrimination Tasks”) as well as the performance subtests-Motor Skills and Visual Acuity (collectively the “Cognivue Tasks”). In one aspect of the present invention, the Cognivue Test included either a Three- or Four-Factor structure, explaining 69-75% of the variance. Factor 1 contains all four Memory Tasks while Factor 2 contains the Motor Skills (Adaptive Motor) and Visual Salience (Visual Acuity) subtests. If using a Three-Factor structure, all four of the Discrimination Tasks are grouped together into Factor 3. If using a Four-Factor structure, the Shape Discrimination and Motion Discrimination tasks comprise Factor 4a while the Letter Discrimination and Word Discrimination tasks comprise Factor 4b. One or more of the Cognivue Tasks may be described in greater detail in one or more of U.S. Pat. Nos. 8,986,015; 8,979,545; 8,882,510; 8,562,541; 8,475,391; 8,454,165; and 8,257,140, the entirety of each being incorporated herein by reference.

It should be noted that the results of the Bio-Hermes study reproduced nearly identical data to that received from the earlier-performed FOCUS study. As one skilled in the art may know, the FOCUS study refers to the Further the Objective and Clinical Understanding of Cognivue Study initiated by Applicant to demonstrate the applicability of the Cognivue Test in the general population, including across age ranges, by sex, and over levels of education. The Cronbach's alpha for the Bio-Hermes study is 0.879 (95% CI: 0.867-0.890), with the inter-item correlation matrix almost identical to prior analyses, including the FOCUS study. Thus, the data shows that the Cognivue Test psychometric properties are consistent across studies such that the Bio-Hermes study results utilizing the Cognivue Test provide reliable screening metrics for identifying cognitive impairment.

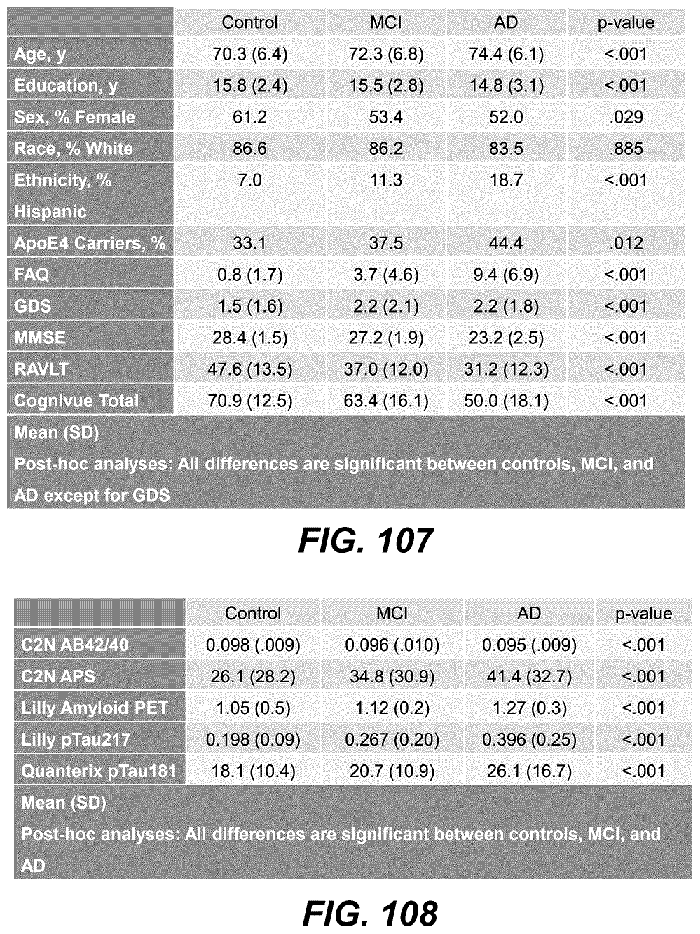

As shown in FIG. 107, the mean age of the individuals completing the Bio-Hermes study was 72.0+6.7 y (Range: 59-85), with a mean education of 15.4+7.8 y (Range: 2-24), 56.2% were female, and 37.5% were ApoE4 carriers. The sample was 75.6% Non-Hispanic White, 11.3% Non-Hispanic Black, and 11.4% Hispanic. The diagnostic categories, as determined by traditional methodologies, were 41.6% healthy controls, 31.1% MCI, and 27.3% Probable AD. The mean Mini-Mental State Examination (“MMSE”) score was 26.6+2.9 (Range: 13-30), and mean RAVLT Total Score was 39.9+14.5 (Range: 0-67), the mean Functional Activities Questionnaire (“FAQ”) score was 4.0+5.7 (Range; 0-30), and the mean Global Deterioration Scale (“GDS”) score was 1.9+1.9 (Range: 0-17). The mean Cognivue Test Score was 62.9+17.4 (Range: 0-93).

Biomarker differences between diagnostics groups (Control, MCI, AD) within the Bio-Hermes study were examined and compiled within the table shown in FIG. 108. The Bio-Hermes study cohort shows expected differences across diagnostic groups. Cognivue Test scores are significantly different between healthy Controls and MCI groups, as well as between MCI and AD groups. Thus, based on the results shown in FIG. 108, a post-hoc analysis indicates that all differences measured using the Cognivue Test are significant between Controls, MCI, and AD groups.

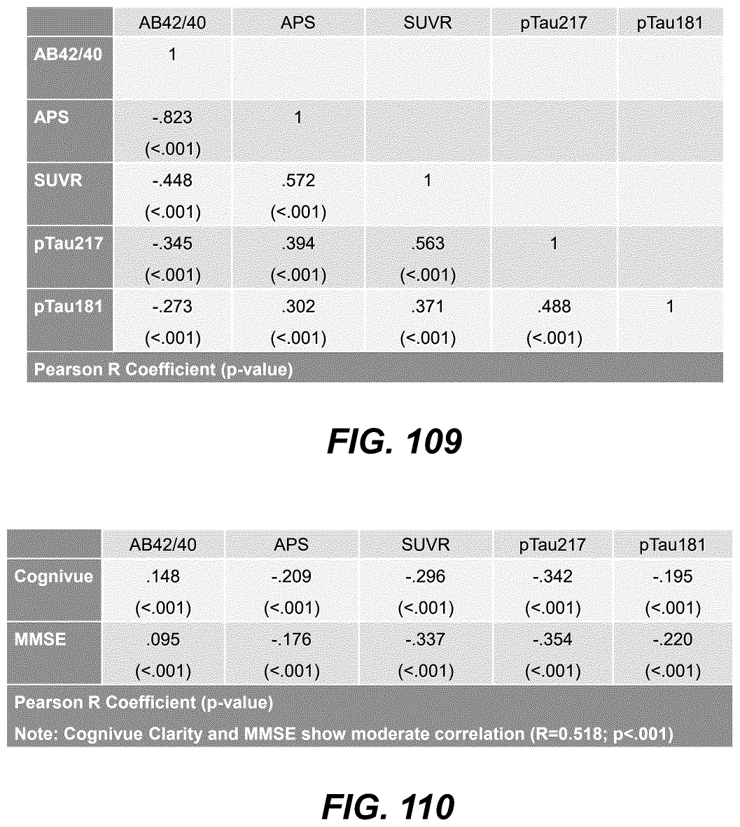

With reference to FIG. 109, the relationship between the different AD biomarkers that characterize amyloid status were examined, including the blood-based AD biomarkers comprising the β-amyloid plasma AB42/40 ratio, the Amyloid Probability Score (APS) (available from C₂N Diagnostics, LLC (St. Louis, MO)), Standardized Uptake Value Ratio (“SUVR”) values obtained from 18F-Florbetapir (Amyvid) PET imaging (available from Eli Lilly), plasma pTau217 (available from Eli Lilly), and plasma pTau181 (available from Quanterix Corp. (Billerica, MA)). As has been recognized in the art, both amyloid and tau markers are reported to be highly associated with, and may be predictive of, neuritic plaque deposition in the AD brain.

As seen from FIG. 109, the AD biomarkers selected through interrogating the Bio-Hermes study data show moderate correlations with each other except for C₂N AB42/40 and APS (of which AB42/40 is part). There are also stronger relationships between the amyloid markers and the pTau217 marker than with pTau181 marker.

Turning now to FIG. 110, the strength of association between AD biomarkers and two cognitive screening tests—the MMSE and the Cognivue Test—is shown. As seen in FIG. 110, both the MMSE and the Cognivue Test show low-to-moderate correlation with the AD biomarkers, with Cognivue Test slightly more associated with amyloid measurements and the MMSE slightly more associated with tau measurements.

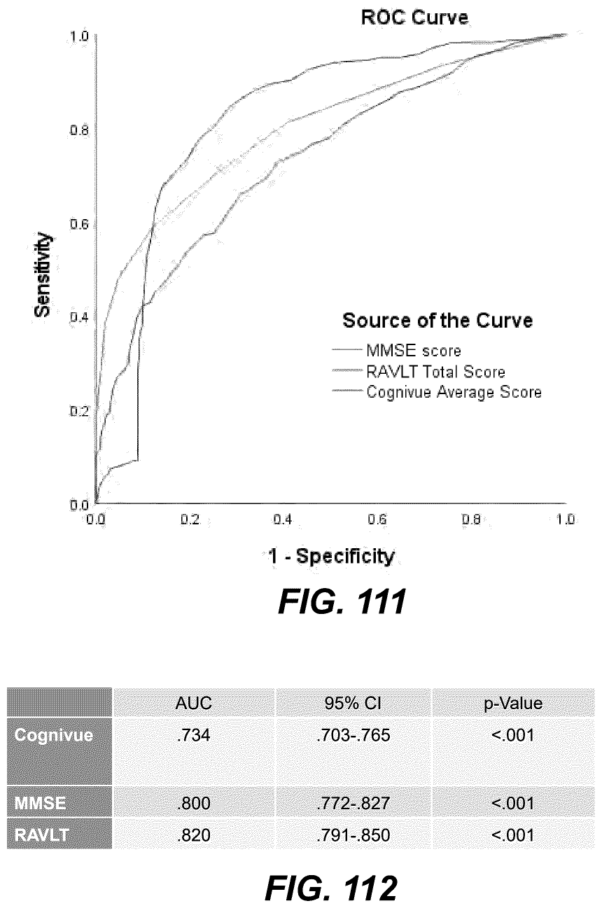

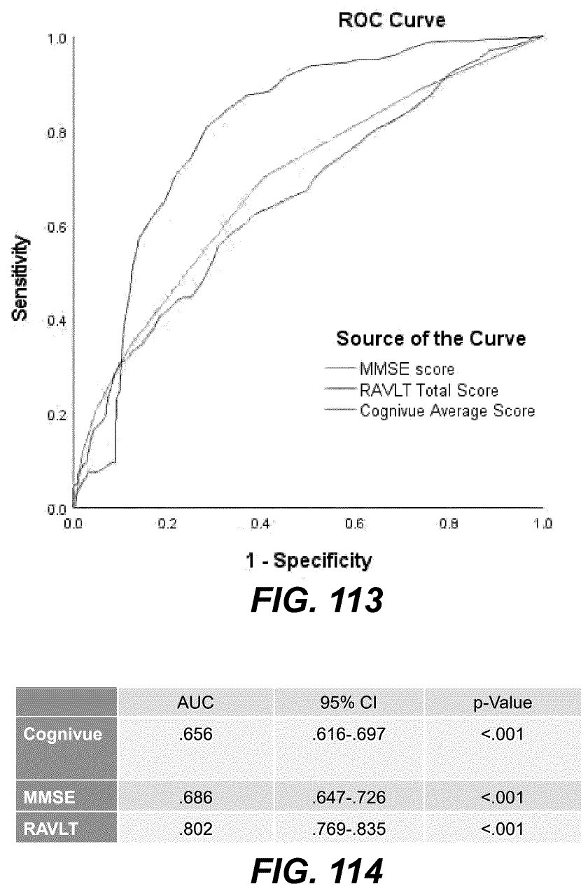

Receiver Operator Characteristic (ROC) curves were generated to examine the respective ability of the Cognivue Test and the MMSE to discriminate between (a) individuals with and without impairment (FIGS. 111 and 112) and (b) healthy controls and MCI (FIGS. 113 and 114). The RVALT (which is not a screening test) was used as a positive control. As seen in FIGS. 111-114, both the Cognivue Test and the MMSE provide modest discrimination between (a) individuals with and without impairment and (b) healthy controls and MCI with overlapping confidence intervals suggesting that no significant differences exist between the two tests. It can also be seen that the RAVLT provided stronger discrimination, particularly between healthy controls and MCI.

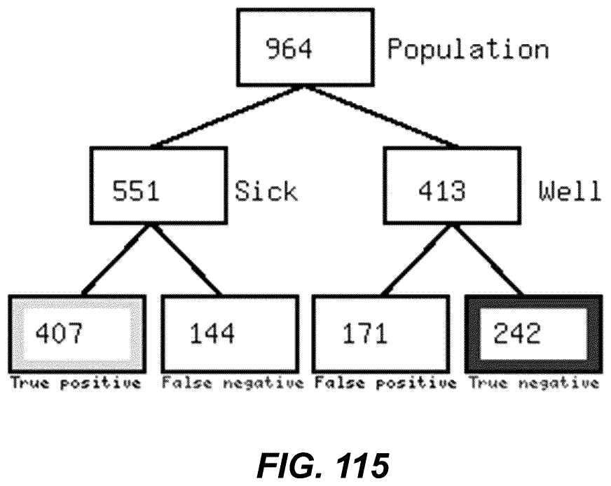

The diagnostic test characteristics of the Cognivue Test were examined to detect and discern individuals with and without cognitive impairments, with the results shown in FIG. 115. Of the 964 individuals (Population) with valid Cognivue Test scores, 413 individuals were identified as healthy Controls (Well), and 551 individuals had a diagnosis of MCI or probable AD (Sick) after using binary diagnostic categories and a Cognivue Test cut-off score of 70 (based on sample distribution of age and scores).

Based on this sample size, the prevalence of impairment was determined to be 57.1%, with a sensitivity of 73.9% and specificity of 58.6%. The Positive Likelihood Ratio is 1.78 (higher scores are better) and the Negative Likelihood Ratio is 0.45 (lower scores are better) provide a Diagnostic Odds Ratio of 3.95. It should also be noted that individuals with Cognivue Test scores less than 70 have nearly a 4-fold increased risk of having MCI or AD.

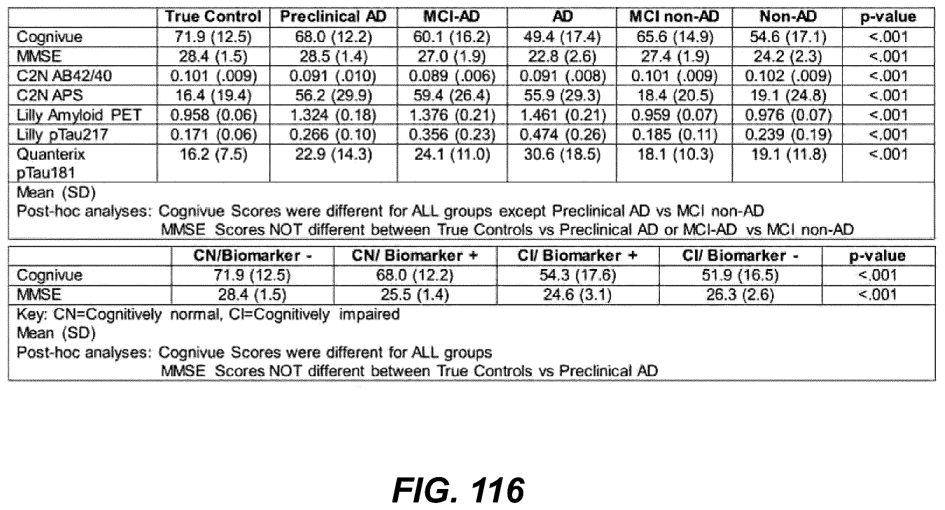

As discussed above, preclinical AD is characterized by normal cognitive performance but with abnormal AD biomarkers (one or more of reduced AB42/40 ratio, increased amyloid PET SUVR, elevated pTau217 and pTau181). To date, it has not been possible to detect Preclinical AD in the absence of biomarkers. Detecting biomarkers requires invasive, and expensive, testing. Thus, what is needed is a clinical screening tool to help identify possible preclinical AD for further follow up, thereby reducing the burden on health care providers while also reducing the unnecessary costs and discomforts experienced by patients. Taking this into view, as the Bio-Hermes study includes both clinical diagnoses and AD biomarkers, the Cognivue Test cohort may be divided into clinical-pathological groups using a SUVR cut-off of 1.1 to examine differences between Cognivue Test scores and cognitive/biomarker status. These results are summarized in FIG. 116. As can be seen, Cognivue scores were different for all groups, while MMSE scores were not different (could not differentiate) between True Controls and Preclinical AD groups.

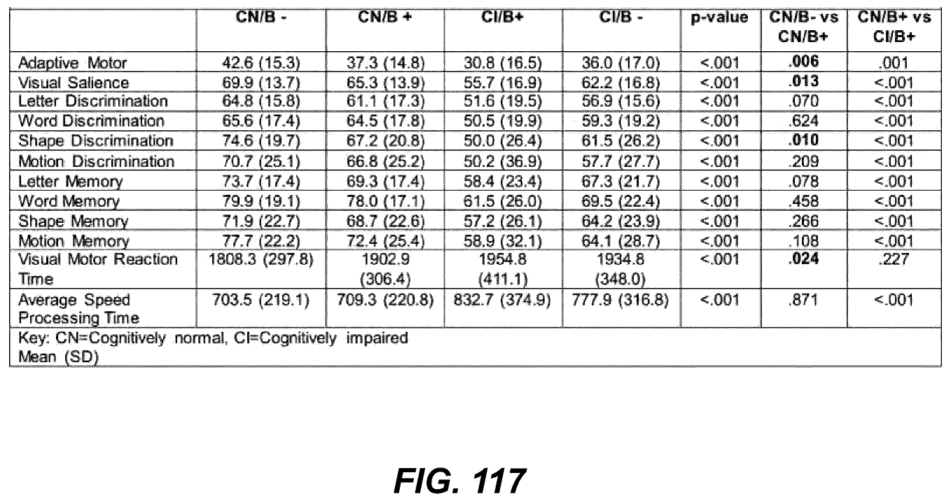

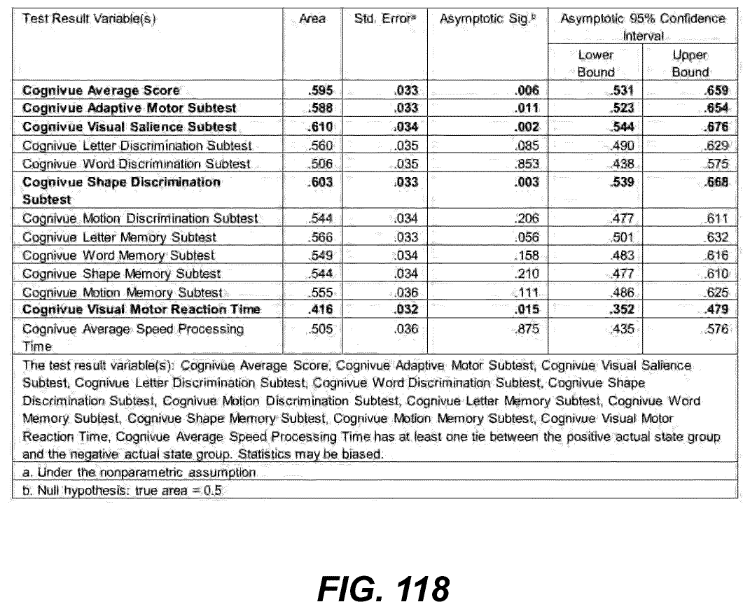

Since the prior analyses suggested that the Cognivue Test could discriminate True Controls from Preclinical AD, the differences between twelve individual Cognivue Test subtests (see, e.g., FIG. 106) and clinical/biomarker status were examined, with the results tabulated as shown in FIG. 117. Discrimination between True Controls from Preclinical AD was also performed using ROC Curves, with the calculated area under the curve results shown in FIG. 118. As one skilled in the art will know, a Receiver Operating Characteristic curve, or ROC curve, is a graphical plot that illustrates the diagnostic ability of a binary classifier system as its discrimination threshold is varied. The ROC curve is the plot of the true positive rate (TPR) against the false positive rate (FPR), at various threshold settings. The ROC curve is thus the sensitivity or recall as a function of fall-out. Given the probability distributions for both true positive and false positive are known, the ROC curve is obtained as the cumulative distribution function (CDF, area under the probability distribution from minus-infinity to the discrimination threshold) of the detection probability in the y-axis versus the CDF of the false positive probability on the x-axis.

As can be seen in FIG. 118, the Cognivue Test subtest scores may be predictive in identifying individuals with Preclinical AD; with particular reference to the tasks/subtest Adaptive Motor Response, Visual Salience, Shape Discrimination, and Visual Motor Reaction Time. Notably, the Preclinical AD group is also different from MCI/AD and non-AD impairment across all tests (except Visual Motor Reaction Time) further indicating that the Cognivue Test may be able to discriminate impaired individuals of any etiology from non-impaired individuals, and may further identify non-impaired individuals who have amyloid deposition (Preclinical AD).

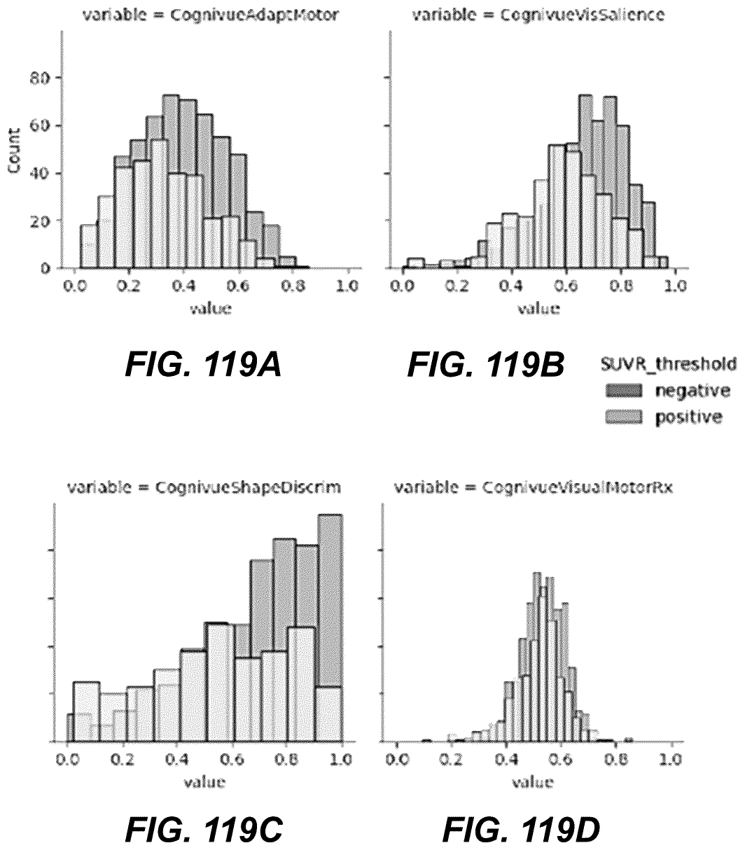

In a further aspect of the present invention, a statistical analysis was conducted to confirm prior findings of significance, along with identifying visual discrimination between negative and positive amyloid determinations, such as defined by a Standardized Uptake Value Ratio (“SUVR”) threshold of 24.1 as shown generally in FIGS. 119A-119D with regard to Adaptive Motor Response (FIG. 19A), Visual Salience (FIG. 19B), Shape Discrimination (FIG. 19C), and Visual Motor Reaction Time (FIG. 19D). With continued reference to FIGS. 119A-119D, it is noted that the markers of amyloid positivity are not clearly discriminable, but patterns between measures in a hyperplane or decision tree ensemble model can be used to highlight separability between negative and positive amyloid determinations.

In accordance with this further aspect, a primary target output is to identify and express an indicator of likelihood of a patient exhibiting amyloid positivity as defined by SUVR threshold, such as but not limited to via a continuous score ranging from 1 to 10, and/or a risk ranking using either 3 or 5 ranks, such as “low/moderate/high” or “very-low/low/moderate/high/very-high”risk. Five methodologies were examined as outlined below:
1) a standardized mean strategy, with scoring based on number of standard deviations (“SD”) s separated from the mean with and without weighting for each measure;
2) linear modeling of centiloids derived from SUVR, with scores thresholded to between 0 and 100, using regression models, such as one or more of linear regression, random forest (RF) regression, support vector (SVM) regression, gradient boosted machine (GBM) regression, with evaluation metrics defined by either the standard SUVR threshold or an alternate threshold;
3) classification modeling of amyloid positivity (meeting SUVR threshold) using random forest (RF) classification, logistic regression, support vector (SVM) classification, and gradient boosted machine (GBM) classification;
4) hyperplane separation using support vector classification coefficients; and
5) ensemble modeling of the combined above models that perform best, with and without weighting of each model.

### Model Development

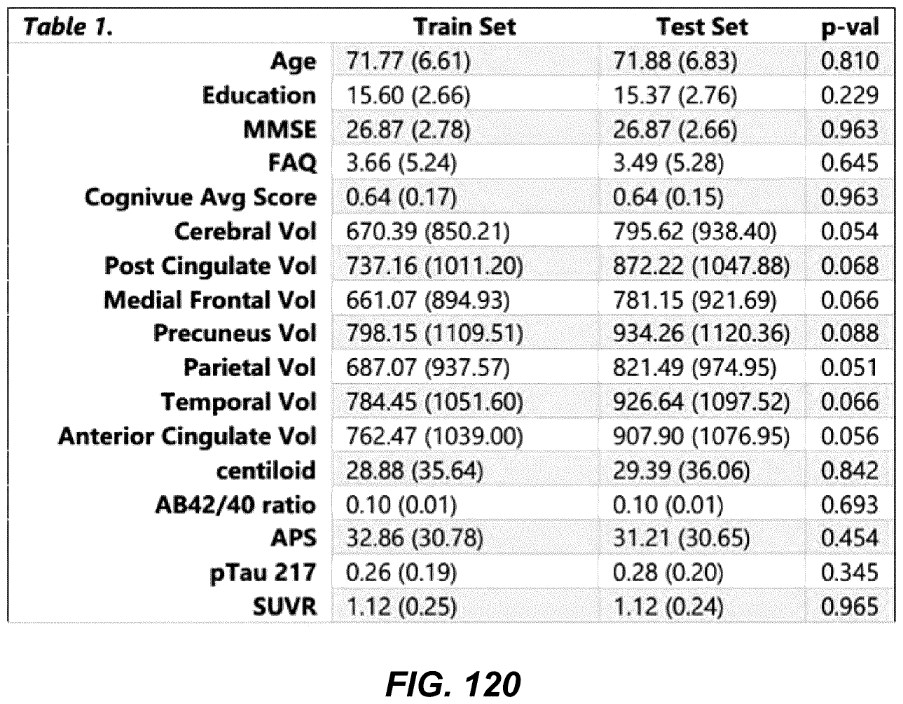

Prior to statistical examination, one-third of the data (stratified based on both SUVR threshold positivity and impairment status) was split into a separate test set and only used in final evaluation. The remaining two-thirds were placed in a train set and used in the model development. Distribution and evaluation of statistical differences using pairwise t-tests is tabulated in FIG. 120. As can be seen in FIG. 120, no significant differences were found between the two sets after applying Bonferroni correction for multiple comparisons. Visual interrogation of the distribution of the data of each measure with respect to amyloid positivity (see FIGS. 119A-119B) led to the formation of the various methodologies/strategies described above and further discussed below.

In the standardized mean strategy (methodology 1), each score that is one or more standard deviations away from the mean is assigned a distinct value (i.e. 1 point for one SD, 2 points for 2 SDs, etc.), with weighting assigned to one or more measures identified via factor analysis and/or K-means clustering. Higher weights led to alternate scoring, e.g. a higher-rated measure is assigned more points for extreme values. A threshold of best classification of amyloid positivity is then determined from the derived score using Youden's J (Youden's index). As there is a minor difference between distributions, if has been found that the standardized mean strategy is easy to implement and scale when new data is collected.

Turning to methodology 2, the linear modeling of centiloids strategy utilizes machine learning regression models, including RF regression, linear regression, GBM regression, and SVM regression, to predict centiloid score using only the identified Cognivue measures (i.e., Visual Motor Reaction Time, Adaptive Motor, Visual Salience, Shape Discrimination). Centiloids were derived from 18F-Florbetapir (Amyvid) PET imaging (available from Eli Lilly) SUVR using the following formula [1], with S signifying SUVR and c signifying Florbetapir centiloid score:

$$c = 183 S - 177 \qquad Formula [ l ]$$

In one exemplary embodiment, the centiloid score was then further refined by applying a lower threshold of 0 (converting all negative numbers to 0) and an upper threshold of 100 (lowering all numbers greater than 100), resulting in a 101-point scale. This final scale was used as the dependent variable in regressions. Regressions were evaluated using error metrics (mean absolute error, root mean squared error) as well as classification metrics using a centiloid threshold of 24.1.

The classification modeling (i.e., methodology 3, above) strategy utilizes logistic regression, RF classification, SVM classification, and/or GBM classification to predict belongingness of each score to either the negative or positive amyloid groups as defined by the SUVR centiloid threshold of 24.1.

Combining the above methodologies for both classification and regression, the hyperplane separation strategy (methodology 4) utilized coefficients derived from a trained SVM classification model to apply weights to each of the identified Cognivue measures (Visual Motor Reaction Time, Adaptive Motor, Visual Salience, Shape Discrimination). Support vector classification works by finding linear separability of groups in a multidimensional space. By extracting the coefficients of the model, the initial inputs can be weighted to then create a linear scale as an output, followed by thresholding the analysis to determine risk. The final score may further be thresholded to identify the best performing value for indication of amyloid positivity.

Lastly, the ensemble modeling strategy (combining methodologies 1 through 4) was used to develop a scale based on the outputs of the best performing models outlined above. Classification output probabilities are assigned 3 points if above 0.8 (80% confidence) and 1 point if above 0.5 (50% confidence). One point is removed if confidence is less than 0.2 (20% confidence). Regressions are further assigned 1 point if above the defined SUVR threshold of amyloid positivity. The hyperplane separation score is also assigned 1 point if above the identified best threshold. Standardized mean is assigned 1 point if above the identified best threshold.

Taking the above into consideration, hyperparameter optimization was performed for all models on ⅔ of the available training set, with the remaining ⅓ of the training set used as an intermediary validation set. Final testing utilized hyperparameters established by the training set.

### Results

Machine Learning-based Feature Selection has revealed that the Cognivue Visual Motor Reaction Time measure contributed less than the other three Cognivue measures (Adaptive Motor, Visual Salience, Shape Discrimination). Accordingly, the Visual Motor Reaction Time measure was dropped from analysis in the interest of parsimony despite being significantly associated with amyloid positivity, as discussed above. It was found that the above-listed models exhibiting the best performance (above 0.60 F1 score) included RF regression, linear regression, GBM regression, and hyperplane separation. As a result, all other strategies were not explored in the following steps.

The above models have been used to assemble the ensemble model so as to generate a linear scale using the following criteria: regressions and hyperplane separation were assigned one point if above their respective identified thresholds, specifically a threshold of 26 for linear regressions, 24.1 (being the standard SUVR threshold) for RF and GBM regressions, and 63 for hyperplane separation; 3 points were assigned if the score was two standard deviations above the respective thresholds, namely 85 for hyperplane separation, 52 for logistic and GBM regressions, and 54 for RF regression. Having established a linear scale of risk for preclinical AD, cutoffs for risk classification were then determined to be 3 points for moderate risk and 6 points for high risk.

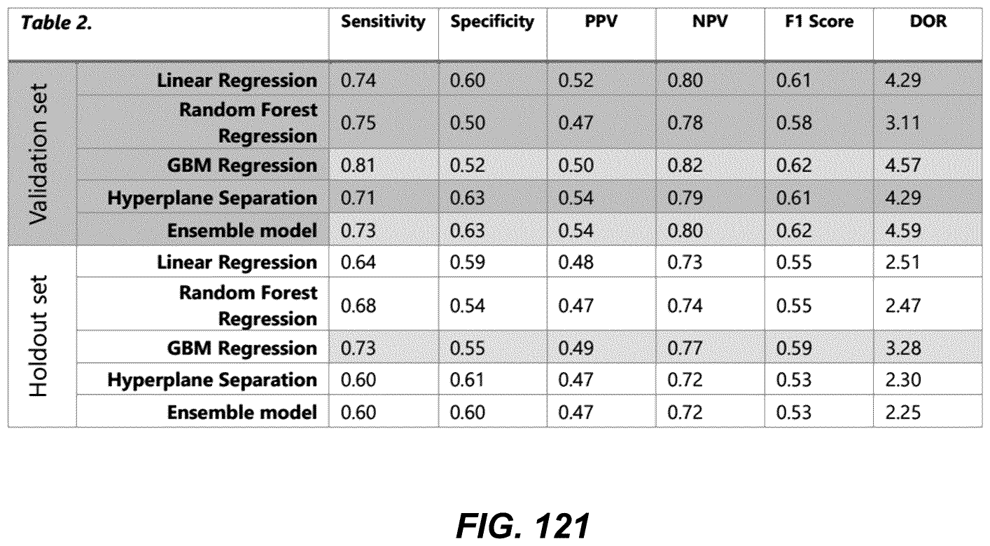

The validation set (⅓ of training set used for validating models) has indicated that the ensemble model was the best performing model (see FIG. 121) with a sensitivity of 72.6%, specificity of 63.4%, and a diagnostic odds ratio (DOR) of 4.59. However, as further shown in FIG. 121, running all models on the holdout test set revealed that the ensemble model was likely overfit, resulting in the lowest available DOR (2.25) and F1 (0.53) scores. Instead, the best performing model on the final test set was the GBM Regression model with a DOR of 3.28 (as highlighted in FIG. 121), which also achieved the second highest score in the validation set. While specificity using GBM regression was consistently lower than the ensemble model, sensitivity was higher. It is noted that greater sensitivity may be potentially more useful than selectivity for this risk measurement paradigm. Thus, the GBM Regression model is used to calculate the Cognivue Amyloid Risk Measure (CARM).

### The Cognivue Amyloid Risk Measure (CARM)

Initially, in creating CARM, thresholds of risk were determined by maximizing sensitivity and positive predictive values (PPV) for stage 3 (high risk); specificity and negative predictive values (NPV) for stage 1 (low risk); and balanced weighted accuracy and f1 scores for stage 2 (moderate risk) based on regression outputs for the GBM Regression model. Final thresholds are as follows:
Low risk: 0-23
Moderate risk: 24-45
High risk: 46-100

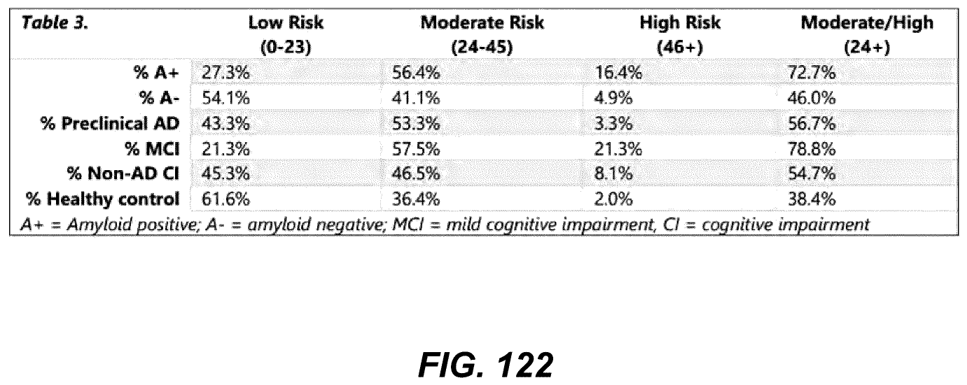

As tabulated in FIG. 122, separating moderate-high risk from low-risk produced a sensitivity of 72.7%, with 80 out of 110 true positives (above SUVR threshold) correctly identified, including 17 of 30 (56.7%) cognitively-normal biomarker-positive patients (1 of which met high risk threshold) and 63 of 80 (78.8%) cognitively-impaired biomarker-positive patients (17 of which met the high risk threshold). In the high risk category, only 9 of 185 biomarker-negative patients (4.9%) were misclassified as amyloid positive.

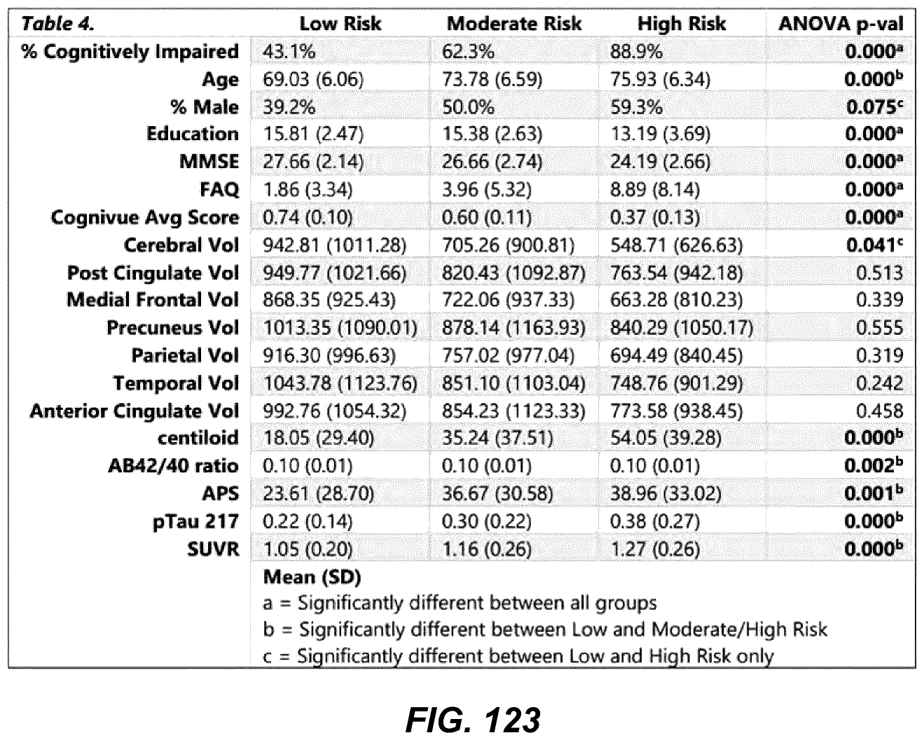

With reference to FIG. 123, independent sample ANOVAs (Analysis of Variance) with Bonferroni-corrected pairwise post hoc tests were examined between the three groups on all other measures available, including diagnostic status, age, sex, years of education, MMSE score, FAQ score, Cognivue Average Score, regional PET SUVR data, AB42/40 ratio, APS, and pTau-217. It is noted that significant differences were found between groups for all measures except Sex and for all regional PET SUVR measures other than cerebral SUVR (although it is noted that cerebral SUVR was only significantly different between the low and high risk stages). Of particular note is that centiloid score was not significantly different between the moderate and high risk stages, indicating that the GBM regression may be capturing additional non-amyloid variance.

Further refinement of CARM was conducted to address improvements in sensitivity, specificity, and overall accuracy in determining amyloid positivity. By way of example and without limitation, three additional CARM paradigms were developed. A second CARM paradigm examined the differing effects of scaling the original centiloid values, as well as different methods of calculating the centiloids. It was noted that unscaling the data entirely (e.g., allowing negative values and values over 100) resulted in worse performance. However, allowing negative values but not values over 100 performed better than the original scaled model (e.g., values between 0-100). A third CARM paradigm examined the addition of additional variables within the model, such as age, sex, race, ethnicity, and education; with age being identified as the most relevant performance variable for determining amyloid positivity. Lastly, a fourth CARM recalibrated CARM so as to be a 4-point scale (discussed below) rather than a 3-point scale (low, moderate, high; as discussed above).

As the risk of amyloid increases with increasing age, age was incorporated within CARM to improve discrimination between amyloid positive and amyloid negative. Also, as centiloid values were allowed to be negative, the SUVR centiloid threshold values were modified to create the 4-point CARM raw score. By way of example, a score of CARM 1 uses a SUVR threshold cutoff of 5 or below which indicates low specificity (below 80% sensitivity); a score of CARM 2 uses a SUVR threshold cutoff of 11.7 or below which indicates good specificity but low sensitivity (above 80% sensitivity but below Youden's J); a score of CARM 3 uses a centiloid threshold cutoff of 22.1 or below which indicates specificity below 80% but above Youden's J; and a score of CARM 4 uses a SUVR threshold above 22.1 which indicates high specificity (greater than 80%).

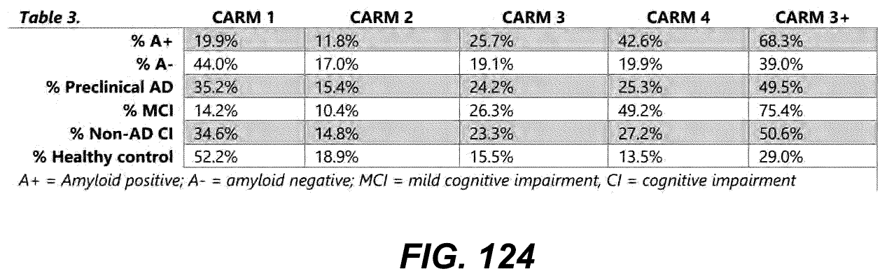

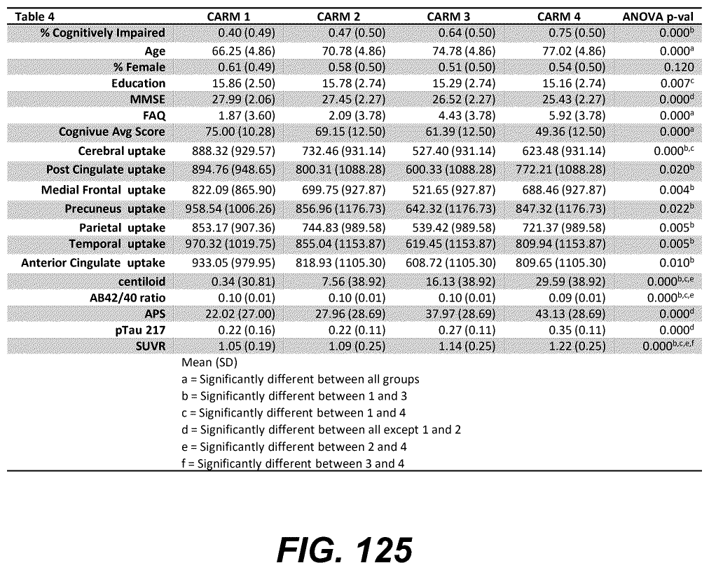

FIG. 124 presents a table compiling CARM data using the above described 4-point scale. As can be seen in FIG. 124, when using CARM 3 as a cutoff (e.g., CARM 3+), 68.3% of biomarker-positive patients were identified, including one-half of the preclinical AD patients. As shown in FIG. 125, independent sample ANOVAs with Bonferroni-corrected pairwise post-hoc tests revealed significant differences between groups in all comparisons except sex. Of note, many regional cortical PET uptake values were found to significantly differ between CARM 1 and CARM 3, likely a result of the placement of MCI and other cognitively impaired patients into the CARM 3 group. CARM 1 and CARM 2 are the most similar to each other, potentially due to the predicted raw CARM values being significantly lower than the centiloid cutoff for amyloid positivity. However, it should be noted that the raw CARM scores, while designed to predict centiloid values, are not useful at directly estimating the centiloid values themselves and thus not useful at determining amyloid positivity. Only the thresholded CARM is powered to do so.

From the above, it can be seen that the generated CARM analyses may be used to identify amyloid positivity as well as allow for discriminating between preclinical Alzheimer's and cognitively normal individuals. It should be noted by those skilled in the art that greater refinement of the GBM regression model, including redetermination of risk thresholds, can be achieved as additional data is collected. It should be further noted by those skilled in the art that additional exploration of integrating vulnerability and resilience indices derived from demographics, medical history, and/or lifestyle, for example, may also lead to improved CARM risk assessment scores.

Thus, taking into consideration the predictive abilities of both the Cognivue Test and the CARM assessment, one aspect of the present invention may allow clinicians or other medical personnel to evaluate a more complete cognitive condition of their patients. By way of example, the Cognivue Test may predictively identify whether a patient exhibits normal cognitive function (NC) or a cognitive impairment (CI), while the CARM assessment may operate to predictively identify amyloid negativity/positivity (A+/A−). As a result, the clinician may segregate a patient into one of four characterizations; i.e., NC/A− indicating normal cognition with little risk of amyloid; NC/A+ indicating normal cognition and high risk of amyloid (preclinical AD); CI/A+ indicating AD; and CI/A+ indicating cognitive impairment with low amyloid risk (non-AD impairment). With this characterization, clinicians and doctors may be able to screen older adults for treatment protocols with anti-amyloid therapies enrich clinical trial recruitment before obtaining expensive biomarkers, and identify individuals likely to have preclinical AD for prevention studies.

Some embodiments of the present disclosure may be used in addition to, instead of, or in combination with visual testing, perform audio, and/or tactile modal testing. In some embodiments, this may further include initial calibration testing. In some embodiments, quantification or diagnosis determination testing may be performed.

Some embodiments of the present disclosure may include diagnosis specific testing. In these embodiments, performance in behavioral tasks, along or in combination with one or more specific degradation techniques, may be indicative of the specific disorder. In these embodiments, behavioral tasks, or behavioral tasks with pre-set degradation may be stored in a database with one or more disorder identifiers.

In some embodiments, broadening diagnosis tests may be included to prevent false positive disorder determination.

In some embodiments, disorder determination may operate in accordance with heuristic models. In these embodiments, known diagnosis may be compared to one or more patient profiles to identify one or more correlation factors from test results. In some embodiments, heuristic models may determine the behavioral task presented, the degradation technique, or combinations thereof. In some embodiments, heuristic models may be self-selecting or refining.

Some embodiments may be configured for detection of input deterioration. In one arrangement, input may be constantly modeled to determine patient tiredness or mobility impairment impact behavioral task response. In some embodiments, detection of input deterioration may initiate suspension of test for a defined time period, and/or provide warning notice to operator.

In some embodiments, system heuristics may formulate the presentation of behavioral tasks, the degradation of stimuli, and combinations thereof. System heuristics may be configured to consider previous patient results, statistical analysis, task priority, and patient demographics, including but not limited, age, weight, medication, etc.

Some embodiments may be configured for one or more simultaneous inputs. For example, one embodiment may be configured, with one or more rotatable wheels, one or more pedals. A further embodiment may be configured with two manipulandum. In this arrangement, tests may be configured to require simultaneous, or coordinated movement across both inputs. In other arrangements, input across multiple input mechanisms may be time delayed, and/or disabled.

In some embodiments, behavioral perception tasks may comprise the presentation of two or more distinct stimuli classes, either concurrently, or at a predefined interval.

Some embodiments may employ an analysis module to assess the impact of dual stimulus presentation and/or dual stimulus tests. In this arrangement, correlation factors system may be identified and compared to diagnosis identifiers.

All references, including publications, patent applications, and patents, cited herein are hereby incorporated by reference to the same extent as if each reference were individually and specifically indicated to be incorporated by reference and were set forth in its entirety herein.

The methods, systems, process flows and logic of disclosed subject matter associated with a computer readable medium may be described in the general context of computer-executable instructions, such as, for example, program modules, which may be executed by a computer. Generally, program modules may include routines, programs, objects, components, data structures, etc. that perform particular tasks or implement particular abstract data types. The disclosed subject matter may also be practiced in distributed computing environments wherein tasks are performed by remote processing devices that are linked through a communications network. In a distributed computing environment, program modules may be located in local and/or remote computer storage media including memory storage devices.

The detailed description set forth herein in connection with the appended drawings is intended as a description of exemplary embodiments in which the presently disclosed subject matter may be practiced. The term “exemplary” used throughout this description means “serving as an example, instance, or illustration,” and should not necessarily be construed as preferred or advantageous over other embodiments.

This detailed description of illustrative embodiments includes specific details for providing a thorough understanding of the presently disclosed subject matter. However, it will be apparent to those skilled in the art that the presently disclosed subject matter may be practiced without these specific details. In some instances, well-known structures and devices are shown in block diagram form in order to avoid obscuring the concepts of the presently disclosed method and system.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein, but is to be accorded the widest scope consistent with the principles and novel features disclosed herein. It is contemplated that additional embodiments are within the spirit and true scope of the disclosed subject matter.

Although the present invention has been described in considerable detail with reference to certain aspects thereof, other versions are possible. Therefore, the spirit and scope of the appended claims should not be limited to the description of the aspects contained herein.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

What is claimed is:

1. A method for accessing the onset or progression of a cognitive impairment within a subject, the method comprising:
a) performing a first test to measure an adaptive motor response of the subject, wherein the first test comprises the following steps executed on a computer processor:
i) presenting an indicium on a GUI;
ii) for at least a first gain, and at least a first noise, moving said indicium, wherein said moving of said indicium comprises:
a) varying acceleration,
b) varying deceleration,
c) varying reversal, and
d) varying speed of movement;
iii) receiving input, via an input mechanism, responsive to said movement of said indicium, determining at least:
a) a reversal latency,
b) an acceleration lag,
c) a deceleration lag, and
d) a speed profile; and
iv) aggregating said reversal latency, said acceleration lag, said deceleration lag, and said speed profile to determine a patient adaptive motor response profile;
b) performing a second test to measure a visual salience of the subject, wherein the second test comprises the following steps executed on the computer processor;
i) presenting a visual stimulus having a pre-set brightness, contrast, background luminance, and spatial frequency composition;
ii) determining a threshold value for each of the brightness, the contrast, the background luminance, and the spatial frequency composition by varying each of the brightness, the contrast, the background luminance, and the spatial frequency composition in a chosen order, individually, or in combination; and
iii) determining a visual saliency profile by aggregating the brightness threshold value, the contrast threshold value, the background luminance threshold value, and the spatial frequency composition threshold value;
c) performing a third test to measure a shape discrimination value of the subject, wherein the third test comprises the following steps executed on the computer processor:
i) presenting at least two stimuli simultaneously such that one of said at least two stimuli comprises a target stimulus and the other one(s) of said at least two stimuli comprises at least one non-target stimulus;
ii) monitoring the speed and accuracy of the subject's indication of a position of said target stimulus as said target stimulus moves along a specific pattern of a visual form, and a signal-to-noise ratio and a stimulus sequence duration of the target stimulus changes;
iii) analyzing the subject's positional error with respect to said position of said target stimulus and observed subject response motion dynamics errors associated with the subject's ability to respond to a movement of said target stimulus;
iv) adjusting the signal-to-noise ratio relating to said target stimulus and non-target stimulus wherein the signal-to-noise ratio is increased until the subject has correctly identified the target stimuli; and
v) creating a shape discrimination score deriving from the subject's accuracy, speed, and precision in responding to said changes in said signal-to-noise ratios and stimulus sequence durations of said at least two stimuli; and
d) evaluating each of the patient adaptive motor response profile of the first test, the visual saliency profile of the second test, and the shape discrimination score of the third test to determine a cognitive assessment where the subject exhibits either a normal cognitive state or cognitive impairment.

2. The method in accordance with claim 1 further comprising:
e) using a machine learning model to evaluate each of the patient adaptive motor response profile of the first test, the visual saliency profile of the second test, and the shape discrimination score of the third test to determine an amyloid assessment wherein the subject exhibits either a negative amyloid determination or an amyloid positivity.

3. The method in accordance with claim 2 wherein the machine learning model employs linear modeling including one or more of linear regression, random forest (RF) regression, support vector (SVM) regression, and gradient boosted machine (GBM) regression.

4. The method in accordance with claim 2 wherein the machine learning model employs classification modeling including one or more of random forest (RF) classification, logistic regression, support vector (SVM) classification, and gradient boosted machine (GBM) classification.

5. The method in accordance with claim 3 wherein the machine learning model further employs classification modeling including one or more of RF classification, logistic regression, SVM classification, and GBM classification.

6. The method in accordance with claim 2 wherein when the cognitive assessment exhibits the normal cognitive state and the amyloid assessment exhibits amyloid positivity, the amyloid positivity is an indication of preclinical Alzheimer's Disease.

7. An apparatus for accessing the onset or progression of a cognitive impairment within a subject, the apparatus comprising:
a) computing device including a memory and a processor;
b) an input device; and
c) a user interface on a computer screen,
wherein the apparatus is configured to:
i) perform a first test to measure an adaptive motor response of the subject, wherein the first test comprises the following steps executed on the processor:
a) presenting an indicium on the user interface;
b) for at least a first gain, and at least a first noise, moving said indicium, wherein said moving of said indicium comprises:
i) varying acceleration,
ii) varying deceleration,
iii) varying reversal, and
iv) varying speed of movement;
c) receiving input, via the input device, responsive to said movement of said indicium, determining at least:
i) a reversal latency,
ii) an acceleration lag,
iii) a deceleration lag, and
iv) a speed profile; and
d) aggregating said reversal latency, said acceleration lag,
said deceleration lag, and said speed profile to determine a patient adaptive motor response profile;
ii) perform a second test to measure a visual salience of the subject, wherein the second test comprises the following steps executed on the processor;
a) presenting a visual stimulus on the user interface having a pre-set brightness, contrast, background luminance, and spatial frequency composition;
b) determining a threshold value for each of the brightness, the contrast, the background luminance, and the spatial frequency composition by varying each of the brightness, the contrast, the background luminance, and the spatial frequency composition in a chosen order, individually, or in combination; and
c) determining a visual saliency profile by aggregating the brightness threshold value, the contrast threshold value, the background luminance threshold value, and the spatial frequency composition threshold value;
iii) perform a third test to measure a shape discrimination value of the subject, wherein the third test comprises the following steps executed on the processor:
a) presenting at least two stimuli simultaneously such that one of said at least two stimuli comprises a target stimulus and the other one(s) of said at least two stimuli comprises at least one non-target stimulus;
b) monitoring the speed and accuracy of the subject's indication of a position of said target stimulus as said target stimulus moves along a specific pattern of a visual form, and a signal-to-noise ratio and a stimulus sequence duration of the target stimulus changes;
c) analyzing the subject's positional error with respect to said position of said target stimulus and observed subject response motion dynamics errors associated with the subject's ability to respond to a movement of said target stimulus;
d) adjusting the signal-to-noise ratio relating to said target stimulus and non-target stimulus wherein the signal-to-noise ratio is increased until the subject has correctly identified the target stimuli; and
e) creating a shape discrimination score deriving from the subject's accuracy, speed, and precision in responding to said changes in said signal-to-noise ratios and stimulus sequence durations of said at least two stimuli; and
iv) evaluate each of the patient adaptive motor response profile of the first test, the visual saliency profile of the second test, and the shape discrimination score of the third test to determine a cognitive assessment where the subject exhibits either a normal cognitive state or cognitive impairment.

8. The apparatus in accordance with claim 7 wherein the apparatus is further configured to use a machine learning model to evaluate each of the patient adaptive motor response profile of the first test, the visual saliency profile of the second test, and the shape discrimination score of the third test to determine an amyloid assessment wherein the subject exhibits either a negative amyloid determination or an amyloid positivity.

9. The method in accordance with claim 8 wherein the machine learning model employs linear modeling including one or more of linear regression, random forest (RF) regression, support vector (SVM) regression, and gradient boosted machine (GBM) regression.

10. The method in accordance with claim 8 wherein the machine learning model employs classification modeling including one or more of random forest (RF) classification, logistic regression, support vector (SVM) classification, and gradient boosted machine (GBM) classification.

11. The method in accordance with claim 9 wherein the machine learning model further employs classification modeling including one or more of RF classification, logistic regression, SVM classification, and GBM classification.

12. The method in accordance with claim 8 wherein when the cognitive assessment exhibits the normal cognitive state and the amyloid assessment exhibits amyloid positivity, the amyloid positivity is an indication of preclinical Alzheimer's Disease.
